SEQUENCE LISTING (84 sequences)

SEQ ID NO: 1 - protein, 14 aa, Artificial Sequence
  peptide binding anti-p24 (HIV-1) antibody CB4-1
GATPEDLNQKLAGN

SEQ ID NO: 2 - protein, 17 aa, Homo sapiens
CRGTGSYNRSSFESSSG

SEQ ID NO: 3 - protein, 15 aa, Artificial Sequence
  peptide binding human cardiac troponin I mAb 11E12
NYRAYATEPHAKKKS

SEQ ID NO: 4 - protein, 10 aa, Artificial Sequence
  Peptide binding a protective anti-GXM mAb 2H1
RYDIEAKVTK

SEQ ID NO: 5 - protein, 8 aa, Homo sapiens
DRVYIHPF

SEQ ID NO: 6 - protein, 12 aa, Homo sapiens
PQSDPSVEPPLS

SEQ ID NO: 7 - protein, 8 aa, Influenza A virus
YDVPDYAS

SEQ ID NO: 8 - protein, 8 aa, Artificial Sequence
  FLAG epitope tag
MDYKAFDN

SEQ ID NO: 9 - protein, 5 aa, Artificial Sequence
  His epitope tag
HHHHH

SEQ ID NO: 10 - protein, 8 aa, Homo sapiens
EPAELTDA

SEQ ID NO: 11 - protein, 8 aa, Homo sapiens
YEVQGEVF

SEQ ID NO: 12 - protein, 8 aa, Homo sapiens
GYSIFSYA

SEQ ID NO: 13 - protein, 12 aa, Homo sapiens
LSEPAELTDAVK

SEQ ID NO: 14 - protein, 16 aa, Homo sapiens
DEPVELTSAPTGHTFS

SEQ ID NO: 15 - protein, 17 aa, Homo sapiens
AGEAAELQDAEVESSAK

SEQ ID NO: 16 - protein, 15 aa, Homo sapiens
LQEPAELVESDGVPK

SEQ ID NO: 17 - protein, 12 aa, Homo sapiens
AQPAELVDSSGW

SEQ ID NO: 18 - protein, 14 aa, Homo sapiens
GLDPTQLTDALTQR

SEQ ID NO: 19 - protein, 10 aa, Homo sapiens
YEVQGEVFTK

SEQ ID NO: 20 - protein, 11 aa, Homo sapiens
HVEVNGEVFQK

SEQ ID NO: 21 - protein, 11 aa, Homo sapiens
SYEVLGEEFDR

SEQ ID NO: 22 - protein, 13 aa, Homo sapiens
QYAVSGEIFVVDR

SEQ ID NO: 23 - protein, 11 aa, Homo sapiens
VYEEQGEIILK

SEQ ID NO: 24 - protein, 11 aa, Homo sapiens
LYEVRGETYLK

SEQ ID NO: 25 - protein, 8 aa, Homo sapiens
EPVELTSA

SEQ ID NO: 26 - protein, 8 aa, Homo sapiens
DPTQLTDA

SEQ ID NO: 27 - protein, 8 aa, Homo sapiens
VEVNGEVF

SEQ ID NO: 28 - protein, 8 aa, Homo sapiens
YEVLGEEF

SEQ ID NO: 29 - protein, 24 aa, Homo sapiens
HSQPWQVAVYSHGWAHCGGVLVHR

SEQ ID NO: 30 - protein, 33 aa, Homo sapiens
IVGGWECEQHSQPWQAALYHFSTFQCGGILVHK

SEQ ID NO: 31 - protein, 24 aa, Homo sapiens
GSQPWQVSLFNGLSFHCAGVLVDR

SEQ ID NO: 32 - protein, 16 aa, Homo sapiens
NSQPWQVGLFEGTSLR

SEQ ID NO: 33 - protein, 29 aa, Homo sapiens
HECQPHSQPWQAALFQGQQLLCGGVLVGR

SEQ ID NO: 34 - protein, 29 aa, Homo sapiens
EDCSPHSQPWQAALVMENELFCSGVLVHR

SEQ ID NO: 35 - protein, 33 aa, Homo sapiens
VLNTNGTSGFLPGGYTCFPHSQPWQAALLVQGR

SEQ ID NO: 36 - protein, 19 aa, Homo sapiens
LLEGDECAPHSQPWQVYER

SEQ ID NO: 37 - protein, 15 aa, Homo sapiens
PNSQPWQAGLFHLTR

SEQ ID NO: 38 - protein, 22 aa, Homo sapiens
CVTAGTSCLISGWGSTSSPQLR

SEQ ID NO: 39 - protein, 32 aa, Homo sapiens
VMDLPTQEPALGTTCYASGWGSIEPEEFLTPK

SEQ ID NO: 40 - protein, 44 aa, Homo sapiens
RLNPQVRPAVLPTRCPHPGEACVVSGWGLVSHEPGTAGSPRSQG

SEQ ID NO: 41 - protein, 5 aa, Homo sapiens
RLNPQ

SEQ ID NO: 42 - protein, 40 aa, Homo sapiens
RLNPQGDSGGPLVCGGILQGIVSWGDVPCDNTTKPGVYTK

SEQ ID NO: 43 - protein, 8 aa, Homo sapiens
SGWGLVSH

SEQ ID NO: 44 - protein, 8 aa, Homo sapiens
AGWGIVNH

SEQ ID NO: 45 - protein, 8 aa, Homo sapiens
SGWGITNH

SEQ ID NO: 46 - protein, 8 aa, Homo sapiens
SGWGMVTE

SEQ ID NO: 47 - protein, 8 aa, Homo sapiens
WGDVPCDN

SEQ ID NO: 48 - protein, 8 aa, Homo sapiens
WKDVPCED

SEQ ID NO: 49 - protein, 8 aa, Homo sapiens
WNDAPCDS

SEQ ID NO: 50 - protein, 8 aa, Homo sapiens
WNDAPCDK

SEQ ID NO: 51 - protein, 10 aa, Homo sapiens
SFMPNLVPPK

SEQ ID NO: 52 - protein, 14 aa, Homo sapiens
EVDSGNDIYGNPIK

SEQ ID NO: 53 - protein, 13 aa, Homo sapiens
MGAPTLPPAWQPF

SEQ ID NO: 54 - protein, 15 aa, Homo sapiens
MEGISIYTSDNYTEE

SEQ ID NO: 55 - protein, 6 aa, Homo sapiens
RHRRAL

SEQ ID NO: 56 - protein, 36 aa, Homo sapiens
KVLALYNQHNPGASAAPCCVPQALEPLPIVYYVGRK

SEQ ID NO: 57 - protein, 8 aa, Homo sapiens
NHHSPGGS

SEQ ID NO: 58 - protein, 8 aa, Homo sapiens
EPLTILYY

SEQ ID NO: 59 - protein, 8 aa, Homo sapiens
QQHNPAAN

SEQ ID NO: 60 - protein, 8 aa, Homo sapiens
DPLPVRYY

SEQ ID NO: 61 - protein, 8 aa, Homo sapiens
NKHGPGVS

SEQ ID NO: 62 - protein, 8 aa, Homo sapiens
EPLPSQYY

SEQ ID NO: 63 - protein, 6 aa, Homo sapiens
RKKRAL

SEQ ID NO: 64 - protein, 34 aa, Homo sapiens
RVLSLYNTINPEASDCCVSQDLEPLTILYYIGKT

SEQ ID NO: 65 - protein, 8 aa, Homo sapiens
NTLNPEAS

SEQ ID NO: 66 - protein, 8 aa, Homo sapiens
PQDLEPLT

SEQ ID NO: 67 - protein, 8 aa, Homo sapiens
NKLDPEAS

SEQ ID NO: 68 - protein, 8 aa, Homo sapiens
SEDLEPLA

SEQ ID NO: 69 - protein, 8 aa, Homo sapiens
NTANPERS

SEQ ID NO: 70 - protein, 8 aa, Homo sapiens
SQDLDPMA

SEQ ID NO: 71 - protein, 40 aa, Homo sapiens
RSADTTHSTVLGNTLNPEASDCCVPQDLEPLTILYYVGRT

SEQ ID NO: 72 - protein, 8 aa, Homo sapiens
SAHSTHST

SEQ ID NO: 73 - protein, 8 aa, Homo sapiens
NTINPEAS

SEQ ID NO: 74 - protein, 8 aa, Homo sapiens
SSDTTHAS

SEQ ID NO: 75 - protein, 8 aa, Homo sapiens
NKLDPEAS

SEQ ID NO: 76 - protein, 8 aa, Homo sapiens
AAEATHST

SEQ ID NO: 77 - protein, 8 aa, Homo sapiens
NTANPERS

SEQ ID NO: 78 - protein, 39 aa, Homo sapiens
MKWAKNWVLEPPGFLAYECVGTCQQPPEAFNWPFLGPRQ

SEQ ID NO: 79 - protein, 8 aa, Homo sapiens
NWAVDPPG

SEQ ID NO: 80 - protein, 8 aa, Homo sapiens
QPPEAFGF

SEQ ID NO: 81 - protein, 8 aa, Homo sapiens
HWVVSPPG

SEQ ID NO: 82 - protein, 6 aa, Homo sapiens
KPPEAM

SEQ ID NO: 83 - protein, 8 aa, Homo sapiens
NWVRLPPG

SEQ ID NO: 84 - protein, 8 aa, Homo sapiens
QPPEAKKF

(19) United States
(12) Patent Application Publication
(10) Pub. No.: US 2005/0255491 A1
(43) Pub. Date: Nov. 17, 2005

(54) Small molecule and peptide arrays and uses thereof

(76) Inventors: Frank D. Lee, Chestnut Hill, MA (US); Xun Meng, Newton, MA (US); Noubar B. Afeyan, Lexington, MA (US); Neal F. Gordon, Waltham, MA (US)

Correspondence Address: FISH & NEAVE IP GROUP;ROPES & GRAY LLP, ONE INTERNATIONAL PLACE, BOSTON, MA, 02110-2624, US

(21) Appl. No.: 10/989,226

(22) Filed: Nov. 15, 2004

Related U.S. Application Data
(60) Provisional application No. 60/519,530, filed on Nov. 13, 2003.
(60) Provisional application No. 60/532,687, filed on Dec. 24, 2003.

(51) Int. Cl. C12Q 1/68; C07H 21/04; G01N 33/543

(52) U.S. Cl. 435/6; 436/518

(57) ABSTRACT

Disclosed are competition assay methods for reliably detecting the presence and/or quantitation of small molecules (e.g., metabolites) and peptides/proteins in a sample by the use of capture agents specific for immobilized small molecules and/or peptides/proteins. Arrays comprising these small molecules and/or peptides/proteins are also provided.

### REFERENCE TO RELATED APPLICATIONS

This application claims the benefit of filing date of U.S. Provisional application 60/519,530, filed on Nov. 13, 2003; and 60/532,687, filed on Dec. 24, 2003, the entire contents of which are incorporated herein by reference.

### BACKGROUND OF THE INVENTION

Systems biology is a new field in biology that seeks to build from our current knowledge of genetic and molecular function to an understanding of how a whole cell works as a system, and from there, to multicellular systems such as organs and whole animals. While molecular biology has led to remarkable progress in our understanding of biological systems, the current focus of molecular biology is mainly on identification of genes and functions of their products, which are components of the whole biological system. Although systems are composed of such components, the essence of system lies in dynamics, relationship and interaction of system components, and it cannot be described merely by enumerating components of the system. This information must be integrated together to obtain a view of how the whole system works. At the same time, it is misleading to believe that only system structure, such as network topologies, is important without paying sufficient attention to diversities and functionalities of components. Both structure of the system and components plays indispensable role forming symbiotic state of the system as a whole.

To illustrate, while modern medicine has provided a large number of effective drugs for the treatment of many diseases, it is unsettling that we still do not understand how most drugs work in the complex system of whole organism. New drugs often fail after the expenditure of millions of dollars because the effect on a single gene or protein target in the test tube doesn't necessarily have the predicted effect when tested in the human body. A similarly-rooted problem in diagnosis is that individual biomarkers as surrogate end points may not reliably predict clinical outcomes, since such individual biomarkers merely provide a narrow view of the system status, and may not accurately reflect a true correlation to a particular disease condition. Equally unsettling is the fact that we do not quite understand how the cell, or the whole organism work as a whole system, despite the more and more comprehensive knowledge we gain from advanced molecular biology studies of its individual components. On the other hand, it is essential that we know in detail how both genetic mutations and the environment contribute to disease. Answering such questions and solving such problems requires building predictive models of cells, organs, and ultimately, organisms. And this requires not only advanced computational models but the acquisition of new quantitative data, often with new methods capable of interrogating the activity of a large number of genes within whole cells or whole organisms.

Thus one major challenge is to understand at the system level biological systems that are composed of components revealed by molecular biology. Although this may not be the first attempt at system-level understanding, it is the first time in human history that we may be able to understand biological systems grounded in the molecular level as a consistent framework of knowledge. Now is a golden opportunity to uncover the essential principles of biological systems and applications backed up by in-depth understanding of system behaviors. In order to grasp this opportunity, it is essential to establish methodologies and techniques to enable us to understand biological systems in their entirety by investigating, for example, (1) the structure of the systems, such as genes, proteins, metabolism, and signal transduction networks and physical structures, (2) the dynamics of such systems, both quantitative and qualitative analysis as well as construction of theory/model with powerful prediction capability, (3) methods to control systems, and (4) methods to design and modify systems for desired properties.

This systematic approach will have major impacts in a wide variety of research and development fields, including predictive, preventive and personalized medicine. Quantitative understanding of all components of an entire subcellular, cellular, or organism system, at least an important subset thereof, and their responses to external (environmental, medical, etc.) and internal (e.g., pathological) perturbations could also dramatically speed up identification of biomarkers as surrogate end points, drug discovery, side effect elimination, etc., by allowing one to predict the effects of attacking specific targets within the context of the complex cellular circuits.

The system biology approach is based on comprehensive acquisition, storage, and analysis of a large amount of data spanning genome, transcriptome, proteome, and metabolome.

In the past, DNA microarrays alone have shown promise in advanced medical diagnostics. Several groups have shown that when the gene expression patterns of normal and diseased tissues are compared at the whole genome level, patterns of expression characteristic of the particular disease state can be observed. Bittner et al., (2000) Nature 406:536-540; Clark et al., (2000) Nature 406:532-535; Huang et al., (2001) Science 294:870-875; and Hughes et al., (2000) Cell 102:109-126. For example, tissue samples from patients with malignant forms of prostate cancer display a recognizably different pattern of mRNA expression to tissue samples from patients with a milder form of the disease. c.f., Dhanasekaran et al., (2001) Nature 412 (2001), pp. 822-826.

Monitoring key proteins directly in blood, sputum or urine samples, etc., using, for example, protein-based arrays, is another attractive approach, since proteins are really the “actors in biology” (see “A Cast of Thousands” Nature Biotechnology March 2003). It is reasonable to believe that the body would react in a specific way to a particular disease state and produce a distinct “biosignature” in a complex data set, such as the levels of 500 proteins in the blood. This has sparked great interest in the development of devices such as protein-detecting microarrays (PDMs) to allow similar experiments to be done at the protein level, particularly in the development of devices capable of monitoring the levels of hundreds or thousands of proteins simultaneously. Past efforts have focused on overcoming certain technical difficulties in generating PDMs, including target reagents and detection agents generation, comprehensive coverage of all possible proteins (including splicing variants, or membrane-bound proteins) in an organism, and sample preparation methods suitable for array applications. Current detection methods are either not effective over all proteins uniformly or cannot be highly multiplexed to enable simultaneous detection of a large number of proteins (e.g., >5,000), due to, for example, limitations of various detection methods, protein complex formation, and the presence of autoantibodies which affect the outcome of immunoassays in unpredictable ways, e.g., by leading to analytical errors (Fitzmaurice T. F. et al. (1998) Clinical Chemistry 44(10):2212-2214). For example, prostate specific antigen (PSA) is known to exist in serum in multiple forms including free (unbound) forms, e.g., pro-PSA, BPSA (BPH-associated free PSA), and complexed forms, e.g., PSA-ACT, PSA-A2M (PSA-alpha₂-macroglobulin), and PSA-API (PSA-alpha₁-protease inhibitor) (see Stephan C. et al. (2002) Urology 59:2-8). Similarly, Cyclin E is known to exist not only as a full length 50 kD protein, but also in five other low molecular weight forms ranging in size from 34 to 49 kD. In fact, the low molecular weight forms of cyclin E are believed to be more sensitive markers for breast cancer than the full length protein (see Keyomarsi K. et al. (2002) N. Eng. J. Med. 347(20):1566-1575).

On the other hand, metabolic profiling is emerging as a powerful technology with the capability to rapidly enhance our understanding of fundamental biological problems. Plant metabolic profiling has one of its origins in the area of herbicide target development. During the 1980s, GC profiles of simple extracts of herbicide treated barley plants yielded enormous amounts of information, based on which a simple analysis of response profiles of known and unknown peaks was sufficient to group herbicides according to their mode of action. This approach was later adopted and extended for the analysis of transgenic plants, which necessitates a fast, broad and open analysis of plant metabolism following the creation of transgenic lines. In response, GC/MS based profiling method was used in numerous studies to provide a rapid snapshot of the status of metabolism in transgenic plants to study the complexity of plant metabolism, and the power of this approach for phenotyping has now been clearly demonstrated in the scientific literature. Although these studies deals with plant subjects, there is no reason to believe that the same technology cannot be used in other setting, such as in animal samples or environmental samples. In fact, cellular metabolism, the integrated inter-conversion of hundreds of metabolic substrates through enzyme-catalyzed biochemical reactions, is perhaps the most studied example of the complex intracellular web of molecular interactions. While the topological organization of metabolic networks is increasingly well understood, the dynamic principles governing their activities remain largely unexplored.

In the last few years, technologies such as metabolic profiling have come under scrutiny for their potential utility in functional genomics, hence, the emergence of the term “metabolomics,” together with functional genomics companies with their missions focusing on the identification of gene function through the application of metabolite profiling technologies.

Metabonomics, or metabolite profiling, measures the real outcome of the potential changes suggested by genomics and proteomics. It describes the direct result of the integrated biochemical status, dynamics, interactions, and regulation of whole systems or organisms at a molecular level. Systems biology approaches present a different and broader perspective from the discrete, relatively static measurements of the past. As such, they offer new understanding of disease processes and targets and of the beneficial and adverse effects of drugs, but they also bring new challenges. Exploitation of patterns rather than single indicators, and the dynamic nature of metabonomics end-points, suggest a dose-response continuum and perhaps challenge both industry and regulators with the obsolescence of the crude no-effect dose/effect dose concept. Characterization of individual amenability to therapy and susceptibility to toxicity (“pharmacometabonomics”) has economic and ethical implications. These opportunities and challenges are to be explored in the context of the present and future roles of metabonomics in drug development.

For example, biomarkers that validate pathological/physiological status may contribute to pharmacometabolomics studies by ensuring appropriate classification of subjects, and to drug development studies by identifying metabolites and profiles that differ between two or more states of interest. A serum profile that reflects changes in, for example, caloric intake or levels of certain metabolites in diseased verses normal subjects will be of great interest for diagnosis and drug discovery.

Modern, high-throughput assay technologies have enabled metabolic profiling at much higher resolution and scale than possible so far. Similar to developments in RNA and protein expression profiling, computational data mining and functional inference are required to extract the valuable information contained in these data and integrate them into predictive models. In particular, such large-scale data can provide sample numbers that statistically support the complex, combinatorial, and nonlinear interactions that the most advanced association mining methods now uncover (e.g., GeneLinker™ Platinum).

Metabolic profiles of bodily fluids such as plasma, cerebrospinal fluid and urine reflect both normal variation and the physiological impact of disease and pharmaceuticals on organ systems. Hundreds to thousands of low-molecular-weight metabolites have been tracked and quantified in these body fluids collected from healthy and diseased populations, using technology platforms for large-scale metabolic profiling such as GC-MS and LC-MS. This approach can be applied to clinical studies of many common diseases such as multiple sclerosis (MS) and rheumatoid arthritis (RA).

Other technology platforms, such as fast gradient HPLC with parallel coulometric array electrochemical, and MS detection for redox metabolic profiling have been used to obtain pg sensitivity, 10⁸ dynamic response range and chemical structure information for multivariate study of redox active small molecules. The importance of biological redox reactions to disease, therapeutic action, metabolism and toxicity provide this combined detection approach with the advantages of applicability to a mechanistically targeted subset of the metabolome. Metabonomic toxicity studies, using exploratory pattern recognition analysis of urinary metabolite profiles obtained from animals receiving a variety of xenobiotic compounds, have demonstrated consistent differentiation from control groups and structural characterization of potential markers of toxicity.

Still other technology platforms, such as Fourier transform infrared (FT-IR) spectroscopy as a high-throughput (1 second is typical per sample), “holistic”, metabolic fingerprinting screening approach, and flow-injection, electrospray ionization, mass spectrometry (FI-ESI-MS), have been successfully used in metabolic profiling.

Advanced as these technology platforms (LC-MS/MS, NMR, and FT-MS) are, there are some unfortunate common drawbacks for these technologies, including: 1) all need expensive instruments, which may not be easily accessible, especially for small academic or biotechnology companies, and are expensive to operate and maintain even for large companies; 2) relatively low to medium throughput, which hampers large-scale genome-wise analysis; 3) complicated sample processing steps. In addition, these methods tend to provide a very complex picture of all detectable metabolites and proteins, no matter whether or not these metabolites or proteins are actually relevant to the condition being studied. In fact, undiscriminated accumulation of large amount of such data may even obscure the most useful information, making it more difficult to discern the useful patterns/profiles associated with a specific condition.

Thus there is a need for assays that are relatively inexpensive, high throughput, preferably useable with easy sample processing steps, and that can detect multiple analytes (DNA, RNA, protein, small metabolites) either individually or simultaneously.

### SUMMARY OF THE INVENTION

One aspect of the invention provides a method for quantitating a plurality of target analytes in a sample, comprising: (1) immobilizing said plurality of target analytes and/or unique derivatives thereof to a support, said unique derivatives, if used, predictably result from a treatment of said plurality of target analytes within said sample; wherein each of said plurality of target analytes or unique derivatives thereof is immobilized on a series of distinct addressable locations on said support; (2) for each of said plurality of target analytes or unique derivatives thereof, generating one or more capture agents that specifically bind said target analytes or said unique derivatives thereof; (3) optionally, subjecting said sample to said treatment; (4) contacting said plurality of target analytes or unique derivatives thereof on said support to a series of control samples, each within one of the series of distinct addresable locations, and each comprising a mixture of a fixed concentration of said capture agents and a variable concentration of said target analytes or unique derivatives thereof in solution; (5) generating a standard competition curve for each said plurality of taregt analytes, by measuring the amount of said capture agents bound to said target analytes or unique derivatives thereof on said support; (6) contacting said plurality of target analytes or unique derivatives thereof on said support to a mixture of said fixed concentration of said capture agent and said sample, in one of the series of distinct addressable locations, optionally after said treatment in step (3); (7) determining the concentration of each said plurality of target analytes, using each of said standard competition curves, by measuring the amount of said capture agent bound to said target analytes or unique derivatives thereof on said support.

In one embodiment, the plurality of target analytes or derivatives thereof include 5, 10, 20, 50, 100, 500, 1000, 2000, 5000, 10000 or more members.

In one embodiment, in step (1), said plurality of target analytes or derivatives thereof are immobilized on more than one distinct addressable locations on said support.

In one embodiment, each of said more than one distinct addressable locations contains a different amount of immobilized said target analytes or derivatives thereof.

In one embodiment, the target analytes are small molecules, each independently of molecular weights of about 50-5000 Da, 50-4000 Da, 50-3000 Da, 50-2000 Da, 50-1000 Da, 50-500 Da, 50-200 Da, or 50-100 Da.

In one embodiment, the small molecules comprises metabolites.

In one embodiment, the metabolites are surrogate markers or potential surrogate markers of a disease or a condition.

In one embodiment, the disease is multiple sclerosis (MS), rheumatoid arthritis (RA), neoplastic, cardiovascular, neurodegenerative, renal, or hepatic disease.

In one embodiment, the condition is exposure to toxic agent (e.g., pesticide, environmental toxin, bacterial toxin), drug candidate, nutritional agent, or allergen.

In one embodiment, the target analyte is a protein, said derivative is a PET sequence of said protein.

In one embodiment, the PET sequence is identified by computationally analyzing amino acid sequence of said target analyte, including a Nearest-Neighbor Analysis that identifies unique amino acid sequences based on criteria that also include one or more of pI, charge, steric, solubility, hydrophobicity, polarity and solvent exposed area.

In one embodiment, the plurality of target analytes comprise both small molecule and protein.

In one embodiment, the small molecule and protein are surrogate markers or potential surrogate markers of a disease or a condition.

In one embodiment, the disease is selected from multiple sclerosis (MS), rheumatoid arthritis (RA), a neoplastic disease, a cardiovascular disease, a neurodegenerative disease, a renal disease, or a hepatic disease

In one embodiment, the method further comprises determining the specificity of each of said capture agent generated in (2) against one or more structurally similar analogs (e.g., nearest neighbors), if any, of said target analyte.

In one embodiment, competition assay is used in determining the specificity of said capture agent generated in (2) against said structurally similar analogs.

In one embodiment, the method further comprises determining the specificity of each of said capture agent generated in (2) using a proteome matrix array.

In one embodiment, the proteome matrix array comprises polypeptides representing each and every protein wthin the sample.

In one embodiment, the proteome matrix array comprises polypeptides representing the top 100, 300, 500, or 1000 most abundantly expressed proteins within the sample.

In one embodiment, the proteome matrix array excludes excessively hydrophobic peptides, short peptides of no more than 5 residues, or long peptides of no less than 50 residues.

In one embodiment, all peptides on said proteome matrix array have the same concentration.

In one embodiment, each peptide on said proteome matrix array has a concentration proportional to its concentration in the sample.

In one embodiment, the specificity value S for at least 50% of all of said capture agents is no more than about 0.5, 0.4, 0.3, 0.2, 0.1, preferably no more than about 0.05, 0.02, or 0.01.

In one embodiment, the capture agent is a full-length antibody, or a functional antibody fragment selected from: an Fab fragment, an F(ab′)2 fragment, an Fd fragment, an Fv fragment, a dAb fragment, an isolated complementary determining region (CDR), a single chain antibody (scFv), or derivative thereof.

In one embodiment, the capture agent is nucleotides; nucleic acids; PNA (peptide nucleic acids); proteins; peptides; carbohydrates; artificial polymers; or small organic molecules.

In one embodiment, said capture agent is aptamers, scaffolded peptides, or small organic molecules.

In one embodiment, said treatment is denaturation and/or fragmentation of said sample by a protease, a chemical agent, physical shearing, or sonication.

In one embodiment, the denaturation is thermo-denaturation or chemical denaturation.

In one embodiment, the thermo-denaturation is followed by or concurrent with proteolysis using thermo-stable proteases.

In one embodiment, the thermo-denaturation comprises two or more cycles of thermo-denaturation followed by protease digestion.

In one embodiment, the fragmentation is carried out by a protease selected from trypsin, chymotrypsin, pepsin, papain, carboxypeptidase, calpain, subtilisin, gluc-C, endo lys-C, or proteinase K.

In one embodiment, the protease is immobilized on a solid support.

In one embodiment, the sample is a body fluid selected from: saliva, mucous, sweat, whole blood, serum, urine, amniotic fluid, genital fluid, fecal material, marrow, plasma, spinal fluid, pericardial fluid, gastric fluid, abdominal fluid, peritoneal fluid, pleural fluid, synovial fluid, cyst fluid, cerebrospinal fluid, lung lavage fluid, lymphatic fluid, tears, prostatitc fluid, extraction from other body parts, or secretion from other glands; or from supernatant, whole cell lysate, or cell fraction obtained by lysis and fractionation of cellular material, extract or fraction of cells obtained directly from a biological entity or cells grown in an artificial environment.

In one embodiment, the sample is obtained from human, mouse, rat, dog, monkey or other non-human primates, frog (Xenopus), fish (zebra fish), fly (Drosophila melanogaster), nematode (C. elegans), fission or budding yeast, or plant (A. thaliana).

In one embodiment, the sample is produced by treatment of membrane bound proteins.

In one embodiment, the capture agent is optimized for selectivity for said analyte or derivative thereof under denaturing conditions.

In one embodiment, the amount of capture agents measured in steps (5) and (7), are independently effectuated by using a secondary agent specific for said capture agent, wherein said secondary agent is labeled by a detectable moiety selected from: an enzyme, a fluorescent label, a stainable dye, a chemilluminescent compound, a colloidal particle, a radioactive isotope, a near-infrared dye, a DNA dendrimer, a water-soluble quantum dot, a latex bead, a selenium particle, or a europium nanoparticle.

In one embodiment, the secondary agent is an antibody labeled by an enzyme or a fluorescent group.

In one embodiment, the analyte or derivative thereof is synthesized on said support.

In one embodiment, said analyte or derivative thereof is synthesized or purified before being immobilized on said support

In one embodiment, wherein step (2) is effectuated by immunizing an animal with an antigen comprising said analyte or derivative thereof.

In one embodiment, the derivative is a PET sequence, and the N- or C-terminus, or both, of said PET sequence are blocked to eliminate free N- or C-terminus, or both.

In one embodiment, the N- or C-terminus of said PET sequence are blocked by fusing the PET sequence to a heterologous carrier polypeptide, or blocked by a small chemical group.

In one embodiment, the carrier is KLH or BSA.

In one embodiment, the computationally analyzing amino acid sequence includes a solubility analysis that identifies unique amino acid sequences that are predicted to have at least a threshold solubility under a designated solution condition.

In one embodiment, the PET is 5-10 amino acids long.

In one embodiment, m 33, wherein one or more of said plurality of target proteins are each represented by two or more addressable locations with the same peptide fragment but different amount of said peptide fragment.

Another aspect of the invention provides an array for detecting, profiling or quantitating a plurality of target analytes in a sample, said array comprising a plurality of immobilized target analytes or derivatives thereof on a support, each of said plurality of target analytes is represented by at least one of said plurality of immobilized target analytes or derivatives thereof, said derivatives, if present, predictably result from a treatment of said sample, and each of said plurality of peptide fragments contains a PET unique to said fragments within said sample.

Another aspect of the invention provides a method for characterizing a plurality of candidate antibodies for binding affinity, the method comprising: (1) generating a high density array comprising a plurality of assay chambers, each said chambers contains a plurality of antigens for which said plurality of candidate antibodies are specific, each said antigens are immobilized in said chambers in an addressable location; (2) contacting each said chamber with a solution of said plurality of candidate antibodies; (3) determining the affinity of each of said plurality of candidate antibodies for their respective immobilized antigens by measuring the amount of each of said plurality of candidate antibodies bound to said chamber.

In one embodiment, each of said antigens contains a PET.

In one embodiment, each of said antigens is a small molecule metabolite.

In one embodiment, each of said chamber has 5, 10, 20, 50, 100, or more distinct antigens.

In one embodiment, the solution of said plurality of candidate antibodies contains less than the total numbers of said plurality of peptide antigens in said chamber.

In one embodiment, each said chamber contains the same number of said antigens.

In one embodiment, the amount of any of said antigens is the same in different said chambers.

In one embodiment, each said chambers contains the same number, but proportionally different amounts of immobilized antigens.

In one embodiment, the method further comprises identifying the amount of each of said immobilized antigens that gives rise to the highest apparent antibody affinity.

In one embodiment, each said chamber additionally contains one or more structurally similar analogs (e.g., nearest neighbor peptide antigens) for each said plurality of antigens.

Another aspect of the invention provides an information database comprising: (1) a plurality of PET sequences, and optionally one or more nearest neighbors of each of said PET sequences; (2) property of antibodies specific for each of said PET sequences, said property including affinity towards said PET sequences, specificity towards said PET sequences against all other PET sequences and nearest neighbors, performance of each of said antibodies in one or more in vitro or in vivo assays.

Another aspect of the invention provides a method of designing arrays for large scale profiling of analyte levels for a plurality of target analytes in a sample, the method comprising: (1) generating one or more candidate capture agents specific for each of said target analytes or derivatives thereof; (2) measuring the affinity and cross-reactivity of each of said candidate capture agents to select at least one capture agents with the highest specificity and/or fewest cross-reactivity for each of said target analytes or derivatives thereof; (3) determining, based on the affinity of said at least one capture agents for their respective target analytes or derivatives thereof, and the normal abundance of soluble form of said target analytes or derivatives thereof in said sample, the amount of each of said target analytes or derivatives thereof for immobilization on a support; wherein each said target analytes or derivatives thereof, when immobilized on said support in said amount, and when in contact with said sample, each produces substantially the same amount of binding to its capture agent.

In one embodiment, affinity is measured in step (2) by contacting said candidate capture agents with a concentration series of immobilized target analytes or derivatives thereof against which said candidate capture agents are raised.

In one embodiment, affinity for a plurality of candidate capture agents, each with different specificity, are simultaneously measured in step (2).

In one embodiment, cross-reactivity is measured in step (2) by contacting said candidate capture agents with one or more immobilized structurally similar homologs of target analytes or derivatives thereof against which said candidate capture agents are raised.

In one embodiment, cross-reactivity is measured in step (2) by using a proteome matrix array.

In one embodiment, the proteome matrix array comprises polypeptides representing each and every protein wthin the sample.

In one embodiment, the proteome matrix array comprises polypeptides representing the top 100, 300, 500, or 1000 most abundantly expressed proteins within the sample.

In one embodiment, the proteome matrix array excludes excessively hydrophobic peptides, short peptides of no more than 5 residues, or long peptides of no less than 50 residues.

In one embodiment, all peptides on said proteome matrix array have the same concentration.

In one embodiment, each peptide on said proteome matrix array has a concentration proportional to its concentration in the sample.

In one embodiment, the specificity value S for at least 50% of all of said capture agents is no more than about 0.1, preferably no more than about 0.05, 0.02, or 0.01.

In one embodiment, the method further comprises manufacturing said array by immobilizing each of said target analytes or derivatives thereof in said amount determined in step (3).

In one embodiment, the sample is an undiluted serum sample, or a serum sample diluted by 2, 5, 10, 20, 50, 70, or 100 fold.

Another aspect of the invention provides an array manufactured according to the method of the subject invention.

Another aspect of the invention provides a business method for a biotechnology or pharmaceutical business, the method comprising: (1) designing, using the appropriate subject method, an array with uniform dynamic range of measurements for each of the competent target analytes or derivatives thereof; (2) licensing the right to further develop and/or manufacture said array to a third party.

Another aspect of the invention provides a business method for a biotechnology or pharmaceutical business, the method comprising: (1) designing, using the appropriate subject method, an array of target analytes or derivatives thereof with uniform dynamic range of measurements for each of component said target analytes or derivatives thereof; (2) manufacturing said array for use in diagnostic and/or research experimentation.

In one embodiment, the method further comprises marketing said arrays.

In one embodiment, the method further comprises distributing said arrays.

In one embodiment, the arrays are for use in commercial and/or academic laboratories.

Another aspect of the invention provides a method of screening for marker(s) associated with a condition, said method comprising: (1) immobilizing a plurality of candidate analytes or fragments thereof, each on a series of distinct addressable location, on a support; (2) using competition assay and said immobilized candidate analytes, profiling the level of soluble forms of each of said candidate analytes in a panel of samples with said condition, and in a panel of corresponding control samples without said condition; (3) identifying the candidate analyte(s), if any, as marker(s) associated with said condition, if the levels of soluble forms of said candidate analyte(s) in said panel of samples with said condition are significantly different from the levels of soluble forms of said candidate analyte(s) in said panel of control samples without said condition.

In one embodiment, the marker(s) are biomarkers representing surrogate endpoint(s).

In one embodiment, the condition is a disease condition, a condition associated with a treatment of a disease, or a condition associated with pollution.

In one embodiment, the analytes are small molecules with less than 5000 Da, or 3000 Da, 1000 Da, 500 Da, 100 Da, or 50 Da.

In one embodiment, the analytes are polypeptides, and said fragments are PET-containing peptide fragments.

In one embodiment, the analytes are mixtures of said small molecules of the subject invention and said polypeptides of the subject invention.

In one embodiment, further comprising manufacturing arrays comprising said marker(s) identified in (3).

In one embodiment, levels of each of said marker(s) are statistically significantly different between said samples and said control samples.

In one embodiment, the levels of at least a few of said marker(s) are not statistically significantly different between said samples and said control samples.

Another aspect of the invention provides an array of analytes constructed by the method of the subject invention.

Another aspects of the invention provides a method for quantitating a plurality of target analytes in a sample, comprising: (1) for each of said plurality of target analytes or unique derivatives thereof, generating one or more capture agents that specifically bind said target analytes or said unique derivatives thereof, wherein said unique derivatives, if used, predictably result from a treatment of said plurality of target analytes within said sample; (2) immobilizing said capture agents on a support, wherein each of said capture agent is immobilized on a series of distinct addressable locations on said support; (3) optionally, subjecting said sample to said treatment; (4) providing a mixture of standard analytes labeled with a first agent, each standard analyte has a predetermined concentration, and each standard analyte representing one of said target analytes, wherein all of said target analytes are represented by at least one of said standard analytes; (5) labeling the target analytes in said sample with a second agent; (6) contacting said capture agents to said mixture of standard analytes and said labeled target analytes in (5); (7) measuring the amount of each pair of standard analyte and target analyte bound to their cognate capture agent on said support, thereby determining the amount of each of said target analytes in the sample, and/or the ratio of each target analyte compared to its corresponding standard analyte.

It is contemplated that all embodiments described above, whenever applicable, can be combined with any other embodiments, even those described for a different aspect of the invention.

The method is suitable for use in, for example, diagnosis (e.g., clinical diagnosis or environmental diagnosis), drug discovery, protein sequencing or protein profiling. In one embodiment, at least 50%, 55%, 60%, 65%, 70%, 75%, 80%, 85%, 90%, 95% or 100% of an organism's proteome is detectable from arrayed peptides.

The sample to be tested (e.g., a human, yeast, mouse, C. elegans, Drosophila melanogaster or Arabidopsis thaliana sample, such whole cell lysate) may be fragmented by the use of a proteolytic agent. The proteolytic agent can be any agent, which is capable of predictably cleaving polypeptides between specific amino acid residues (i.e., the proteolytic cleavage pattern). The predictability of cleavage allows a computer to generate fragmentation patterns in sillico, which will greatly aid the process of searching PETs unique to a sample.

The array can be produced on any suitable solid surface, including silicon, plastic, glass, polymer, such as cellulose, polyacrylamide, nylon, polystyrene, polyvinyl chloride or polypropylene, ceramic, photoresist or rubber surface. Preferably, the silicon surface is a silicon dioxide or a silicon nitride surface.

Also preferably, the array is made in a chip format. The solid surfaces may be in the form of tubes, beads, discs. silicon chips, microplates, polyvinylidene difluoride (PVDF) membrane, nitrocellulose membrane, nylon membrane, other purous membrane, non-porous membrane, e.g., plastic, polymer, perspex, silicon, amongst others, a plurality of polymeric pins, or a plurality of microtitre wells, or any other surface suitable for immobilizing small molecules or derivative anchor molecules (such as polypeptide or polynucleotides).

In certain embodiments, the target analyte is a protein or specific fragment thereof. Thus this embodiment of the invention relates to methods and reagents for reproducible protein detection and quantitation, e.g., parallel detection and quantitation, in complex biological samples. Salient features to certain embodiments of the present invention uses PET-based peptide arrays for quantitative measurement of target protein concentration in a sample, using a peptide competition assay.

Methods of the instant invention reduce the complexity of reagent generation, achieve greater coverage of all protein classes in an organism, greatly simplify the sample processing and analyte stabilization process, and enable effective and reliable parallel detection/quantitation, e.g., by optical or other automated detection/quantitation methods, and enable multiplexing of standardized capture agents for proteins with minimal cross-reactivity and well-defined specificity for large-scale, proteome-wide protein detection/quantitation.

Embodiments of the present invention also overcome the imprecisions in detection methods caused by: the existence of proteins in multiple forms in a sample (e.g., various post-translationally modified forms or various complexed or aggregated forms); the variability in sample handling and protein stability in a sample, such as plasma or serum; and the presence of autoantibodies in samples. In certain embodiments, using a targeted fragmentation protocol, the methods of the present invention assure that a binding site on a protein of interest, which may have been masked due to one of the foregoing reasons, is made available to interact with a capture agent. In other embodiments, the sample proteins are subjected to conditions in which they are denatured, and optionally are alkylated, so as to render buried (or otherwise cryptic) PET moieties accessible to solvent and interaction with capture agents. As a result, the present invention allows for detection/quantitation methods having increased sensitivity and more accurate protein quantitation capabilities. This advantage of the present invention will be particularly useful in, for example, protein marker-type disease detection assays (e.g., PSA or Cyclin E based assays) as it will allow for an improvement in the predictive value, sensitivity, and reproducibility of these assays. The present invention can standardize detection/quantitation, and measurement assays for all proteins from all samples.

For example, a recent study by Punglia et al. (N. Engl. J. Med. 349(4): 335-42, July, 2003) indicated that, in the standard PSA-based screening for prostate cancer, if the threshold PSA value for undergoing biopsy were set at 4.1 ng per milliliter, 82 percent of cancers in younger men and 65 percent of cancers in older men would be missed. Thus a lower threshold level of PSA for recommending prostate biopsy, particularly in younger men, may improve the clinical value of the PSA test. However, at lower detection limits, background can become a significant issue. It would be immensely advantageous if the sensitivity/selectivity of the assay can be improved by, for example, the method of the instant invention.

The present invention is based, at least in part, on the realization that exploitation of Proteome Epitope Tags (PETs) present within individual proteins can enable reproducible detection and quantitation of individual proteins in parallel in a milieu of proteins in a biological sample. As a result of this PET-based approach, the methods of the invention detect specific proteins in a manner that does not require preservation of the whole protein, nor even its native tertiary structure, for analysis. Moreover, the methods of the invention are suitable for the detection of most or all proteins in a sample, including insoluble proteins such as cell membrane bound and organelle membrane bound proteins.

The present invention is also based, at least in part, on the realization that PETs can serve as Proteome Epitope Tags characteristic of a specific organism's proteome and can enable the recognition and detection of a specific organism.

The present invention is also based, at least in part, on the realization that high-affinity agents (such as antibodies) with predefined specificity can be generated for defined, short length peptides and when antibodies recognize protein or peptide epitopes, only 4-6 (on average) amino acids are critical. See, for example, Lerner R A (1984) Advances In Immunology. 36:1-45.

The present invention is also based, at least in part, on the realization that by denaturing (including thermo- and/or chemical-denaturation) and/or fragmenting (such as by protease digestion including digestion by thermo-protease) all proteins in a sample to produce a soluble set of protein analytes, e.g., in which even otherwise buried PETs including PETs in protein complexes/aggregates are solvent accessible, the subject method provides a reproducible and accurate (intra-assay and inter-assay) measurement of proteins.

The present invention is also based, at least in part, on the realization that immobilized PET-containing peptides, when properly spaced on a solid support, can facilitate high avidity bidentate binding to their respective antibodies, thus allowing high sensitivity, high specificity protein detection and quantitation using a peptide competition assay.

The present invention is also based, at least in part, on the realization that immobilized PET-containing peptides are highly stable on the solid support, thus allowing the manufacture of long half-life protein array products.

According to one embodiment of this aspect of the present invention a proteolytic agent is a proteolytic enzyme. Examples of proteolytic enzymes, include but are not limited to trypsin, calpain, carboxypeptidase, chymotrypsin, V8 protease, pepsin, papain, subtilisin, thrombin, elastase, gluc-C, endo lys-C or proteinase K, caspase-1, caspase-2, caspase-3, caspase-4, caspase-5, caspase-6, caspase-7, caspase-8, MetAP-2, adenovirus protease, HIV protease and the like.

The following table summarizes the result of analyzing pentamer PETs in the human proteome using different proteases. A total of 23,446 sequences are tagged before protease digestion.

| | Cleavage | Fragment | Tagged |
|---|---|---|---|
| Protease | Site | Length | Proteins |
| Chymotrypsin | after W, F, Y | 12.7 | 21,990 |
| S.A. V-8 E specific | after E | 13.7 | 23,120 |
| Post-Proline | after P | 15.7 | 23,009 |
| Cleaving Enzyme | | | |
| Trypsin | after K, R | 8.5 | 22,408 |

According to another embodiment of this aspect of the present invention a proteolytic agent is a proteolytic chemical such as cyanogen bromide and 2-nitro-5-thiocyanobenzoate. In still other embodiments, the proteins of the test sample can be fragmented by physical shearing; by sonication, or some combination of these or other treatment steps.

An important feature for certain embodiments, particularly when analyzing complex samples, is to develop a fragmentation protocol that is known to reproducibly generate peptides, preferably soluble peptides, which serve as the unique recognition sequences. The collection of polypeptide analytes generated from the fragmentation may be 5-30, 5-20, 5-10, 10-20, 20-30, or 10-30 amino acids long, or longer. Ranges intermediate to the above recited values, e.g., 7-15 or 15-25 are also intended to be part of this invention. For example, ranges using a combination of any of the above recited values as upper and/or lower limits are intended to be included.

The PET may be a linear sequence or a non-contiguous sequence and may be 5, 6, 7, 8, 9, 10, 11, 12, 13, 14, 15, 16, 17, 18, 19, 20, 25, or 30 amino acids in length.

Other features and advantages of the invention will be apparent from the following detailed description and claims.

### BRIEF DESCRIPTION OF THE DRAWINGS

FIG. 1 presents a general scheme for using PET peptide arrays for protein detection and quantitation analysis. A similar scheme may be used for other small molecule metabolites.

FIG. 2 is a schematic drawing of the two assay formats for PET-based peptide competition assay. A similar scheme may be used for other small molecule metabolites.

FIG. 3 illustrates an exemplary embodiment of the PET-based peptide competition assay with immobilized PET peptides. A similar scheme may be used for other small molecule metabolites.

FIG. 4 illustrates the mechanism of the avidity effect in antibody binding to immobilized, properly spaced antigens (e.g., PET peptides, small molecule metabolites, etc.).

FIG. 5 illustrates an exemplary embodiment of the high throughput assay development platform for antibody characterization using the subject arrays (e.g. PET-based peptide array).

FIG. 6 is an illustrative example of the high-density peptide arrays for multiplexing antibody and peptide titration. A similar scheme may be used for other small molecule metabolites.

FIG. 7 shows an exemplary result obtained from a multiplexing antibody titration assay using PET-based peptide arrays. A similar scheme may be used for other small molecule metabolites.

FIG. 8 shows the results of antibody titration curves for the 4 antigens An, Ap, Op and Ur used in FIG. 7.

FIG. 9 demonstrates that the PET-specific antibodies used in FIGS. 7 and 8 are highly specific, and only reacts with different concentrations of the antigens to which they are raised against, but nothing else.

FIG. 10 illustrates the process for PET-specific antibody generation.

FIG. 11 illustrates that PET-specific antibodies are highly specific for the PET antigen and do not bind the nearest neighbors of the PET antigen. The six peptides are represented by SEQ ID NOs: 10, 11, and 25-28.

FIG. 12 illustrates a general scheme of sample preparation prior to its use in the methods of the instant invention. The left side shows the process for chemical denaturation followed by protease digestion, the right side illustrates the preferred thermo-denaturation and fragmentation. Although the most commonly used protease trypsin is depicted in this illustration, any other suitable proteases described in the instant application may be used. The process is simple, robust & reproducible, and is generally applicable to main sample types including serum, cell lysates and tissues.

FIG. 13 provides an illustrative example of serum sample pre-treatment using either the thermo-denaturation or the chemical denaturation as described in FIG. 12.

FIG. 14 shows the result of thermo-denaturation and chemical denaturation of serum proteins and cell lysates (MOLT4 and Hela cells).

FIG. 15 illustrates a general approach to identify all PETs of a given length in an organism with sequenced genome or a sample with known proteome. Although in this illustrative figure, the protein sequences are parsed into overlapping peptides of 4-10 amino acids in length to identify PETs of 4-10 amino acids, the same scheme is to be used for PETs of any other lengths.

FIG. 16 lists the results of searching the whole human proteome (a total of 29,076 proteins, which correspond to about 12 million 4-10 overlapping peptides) for PETs, and the number of PETs identified for each N between 4-10.

FIG. 17 shows the result of percentage of human proteins that have at least one PET(s).

FIG. 18 provides further data resulting from tryptic digest of the human proteome.

FIG. 19 shows a design for the PET-based assay for standardized serum TGF-beta measurement. The peptides are represented by SEQ ID NOs: 55-82.

FIG. 20 illustrates the results of a PET-based peptide competition assay for three representative PET-peptides, PSA-P1, CRP-C1 and CRP-C2.

FIG. 21 illustrates the results of a PET-based peptide competition assay for Troponin T tryptic peptide (represented by SEQ ID NO: 51).

FIG. 22 illustrates that the sample treatment method of the instant invention plays an important role in accurate quantitation of serum protein concentration.

FIGS. 23 and 24 illustrates that the sample treatment method of the instant invention does not cause appreciatable loss of target proteins in the original sample. The peptide is FIG. 23 is represented by SEQ ID NO: 52.

FIG. 25 illustrates the measurement of Survivin concentration using the PET-based peptide competition assay. The peptide is represented by SEQ ID NO: 53.

FIG. 26 illustrates the measurement of CXCR4 concentration using the PET-based peptide competition assay. The peptide is represented by SEQ ID NO: 54.

FIG. 27 illustrates the result of extraction of intracellular and membrane proteins. Top Panel: M: Protein Size Marker; H-S: HELA-Supernatant; H-P: HELA-Pellet; M-S: MOLT4-Supernatant; M-P: MOLT4-Pellet. Bottom panel shows that >90% of the proteins are solublized. Briefly, cells were washed in PBS, then suspended (5×10⁶ cells/ml) in a buffer with 0.5% Triton X-100 and homogenized in a Dounce homogenizer (30 strokes). The homogenized cells were centrifuged to separate the soluble portion and the pellet, which were both loaded to the gel.

FIG. 28 illustrates the structure of mature TGF-beta dimer, and one complex form of mature TGF-beta with LAP and LTBP.

### DETAILED DESCRIPTION OF THE INVENTION

I. Overview

The present invention is directed to methods and reagents for reproducible detection, quantitation and profiling of certain analytes (polypeptides, nucleic acids, and especially small molecule compounds such as lipids, steroids, metabolites), e.g., parallel multiplexing detection and quantitation, in complex biological and non-biological samples. Salient features to certain embodiments of the present invention uses arrays based on certain peptides, small molecules such as metabolites for quantitative measurement of target analyte concentration in a sample, using a competition assay. Such peptide- or small molecule-based arrays may be a mixed array of different types of analytes, including peptides, small molecules, etc. The methods and reagents of the invention allow targeted profiling of a selected group of analytes, especially peptides and small molecules, deemed important for particular purposes, thereby providing a relatively comprehensive view of system status (DNA, RNA, proteins, and/or metabolites) without being burdened by large amounts of trivial and unnecessary data storage and/or analysis.

The methods and reagents of the instant invention can be used, for example, in protein and/or metabolic profiling. Metabolic profiling data can be integrated with genomic and proteomics data, as well as traditional toxicity and clinical measurements, to define complex systems-level responses to various disease conditions, environmental and nutritional factors. The invention provides an important research and diagnostic tool for studying mechanisms of action and identifying biomarkers as surrogate endpoints for numerous diseases including neoplastic, cardiovascular, neurodegenerative, renal and hepatic diseases, as well as markers for monitoring changes in environmental samples.

Methods of the instant invention provide simultaneous profiling of a large spectrum of pre-selected peptide and/or small molecules of interest in a sample, such as candidate biomarkers for the intended purpose. There are several considerations when selecting candidate peptide/small molecule metabolites for array construction. In one respect, each disease condition may be specifically associated with a list of peptides/metabolites, which association is either verified or a strong possibility. Thus in one embodiment, measuring key proteins/metabolites that are simultaneously associated with multiple different disease states may reveal information for several diseases, and therefore command a wider market.

In another respect, many proteins, metabolites and genes are differentially expressed in varied states of biological systems. Some of these analytes vary in a correlated fashion, while the others do not. The ones that do not will likely have additive value in differentiating varied states from ones that are correlated. In other words, there are tightly connected networks of metabolites as well as loosely connected ones in effect in a biological state change. Having one analyte or fifty that all come from a tightly connected network may not be that different in predictive value of system status. And in any event, the fifty from the same network will not likely be very informative as to how other loosely connected networks are affected during such a state change. In other words, discovering the minimal marker set that adequately defines the state of a biological system is probably best done by combining measurements that are maximally additive in their information value in segregating various states. Therefore, there may be a universal set of analytes (e.g., metabolites), the state of which is informative for many different biological states. Thus in certain embodiments, an overlapping sets of maximally informative peptides/small molecules (e.g., serum metabolites) may be selected for immobilizing on an array.

In certain embodiments, where analysis of target peptides are optionally involved, the invention also reduces the complexity of reagent generation to achieve greater coverage of all protein classes in an organism, thereby greatly simplifying the sample processing and analyte stabilization process. This enables effective and reliable parallel detection/quantitation, e.g., by optical or other automated detection/quantitation methods, and enables multiplexing of standardized capture agents for proteins and small molecules with minimal cross-reactivity and well-defined specificity for large-scale, proteome-wide and/or metabolome-wise analyte detection/quantitation.

Embodiments of the present invention provides arrays of immobilized peptides (e.g. PET-based peptides, infra), small molecules, such as metabolites of interest, for simultaneous detection, quantitation, and profiling using competition assays. The present invention also provides methods of using these arrays in drug discovery research (such as drug screening), disease biomarker discovery, pollution monitoring, and environmental sciences.

Related embodiments of the present invention provides mixed arrays of different metabolites, including small molecules and peptides (such as PET-based peptides described in U.S. Ser. No. 60/519,530). This type of array provides simultaneous profiling of different analytes in a single assay, and potentially provides a broader and more complete view for the same purposes above. Data obtained from this types of array provides a means to characterize system responses, to link transcription/translation data to phenotypic responses, and to analyze regulation mechanisms. Instead of predicting the results that would been brought about by the changes in transcription/translation, the array provides actual results of phenotypic responses associated with the changes in transcription/translation.

The present invention is based, at least in part, on the realization that immobilized peptides/small molecule metabolites are highly stable on the solid support, thus allowing the manufacture of long half-life array products.

The present invention is also based, at least in part, on the realization that immobilized analytes, such as peptides or small molecule metabolites, when properly spaced on a solid support, can facilitate high avidity bidentate binding to their respective antibodies, thus allowing high sensitivity, high specificity analyte detection and quantitation using a competition assay format.

The present invention is also based, at least in part, on the realization that by denaturing (including thermo- and/or chemical-denaturation) and/or fragmenting (such as by protease digestion including digestion by thermo-protease as described in U.S. Ser. No. 60/519,530) all proteins in a sample, the subject method provides a reproducible and accurate (intra-assay and inter-assay) measurement of proteins when necessary. An added advantage is that sample complexity is reduced, enabling better detection of non-peptide analytes, such as small molecules.

In certain embodiments, the present invention provides methods, reagents and systems for profiling and quantitating one or more target small molecules within a sample, using the subject small molecule arrays. Briefly, at least one, preferably a panel of elected target small molecules are immobilized on array surface. Capture agents specific for these small molecule targets are raised for use in a competition assay format, in which a standard competition curve is generated using the capture agents and a series of different concentrations of competitor small molecule targets in solution. Once the standard competition curve is generated with a series of known concentrations of small molecule targets, the concentration of the small molecule targets in any given sample (optionally pre-treated as described below) can be readily determined using the competition assay.

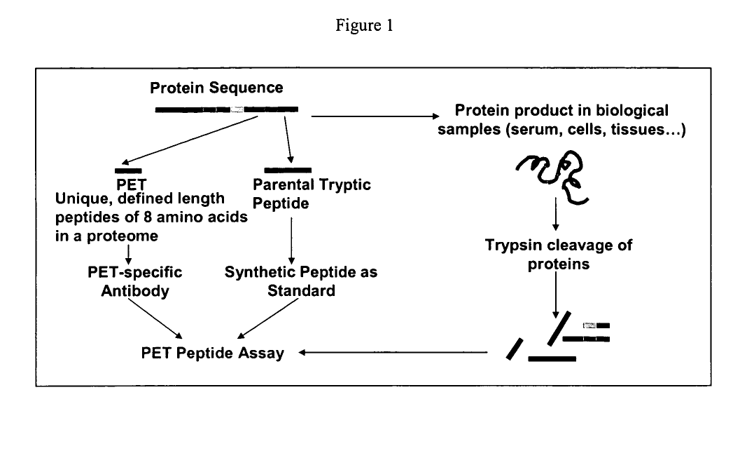

The present invention provides methods, reagents and systems for quantitating one or more target proteins within a sample, by PET-based peptide arrays. FIG. 1 presents a general scheme for using PET peptide arrays for protein detection and quantitation analysis, which may be adapted for use of any other small molecule metabolites. Briefly, for any given target protein sequence, at least one PET (such as a commonly used 8-mer PET) unique in the proteome is identified. This PET sequence can then be used to raise capture agents specific for the PET, such as a PET-specific antibody (see below). Meanwhile, a parental peptide fragment resulting from a pre-determined treatment, such as trypsin digestion, can be generated in silico or synthesized in vitro for use in standard competition curve construction. Once the capture agent and the peptide fragment are available, and the standard curve is generated, the concentration of the target protein in any given sample (preferably pre-treated as described below) can be readily measured using the PET peptide-dependent competition assay. In the case of small molecules, other than the PET-peptide identification step, all other steps are essentially identical.

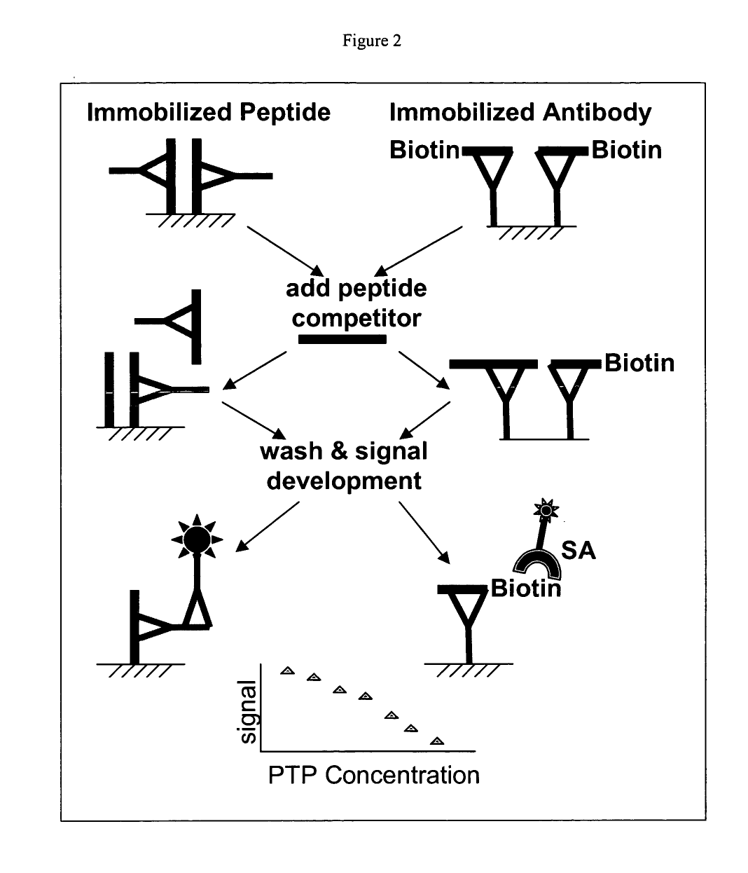

There are at least two formats of the array that can be used in competition assays for analyte concentration measurement. FIG. 2 uses PET-based array as an illustration.

In one embodiment (the PET peptide array), the method utilizes an array of peptide fragments immobilized on a support, the array comprising a plurality of peptide fragments, each of which represents one unique target protein within the sample. The peptide fragments each contain a PET sequence unique within the sample. When such an array is in contact with a mixture of capture agents specific for the immobilized peptides, the capture agents will specifically bind to their respective immobilized peptide fragments. Ideally, each capture agent only binds the peptide against which the capture agent is raised, but not any other peptides on the same array (e.g., no cross-reactivity). However, if soluble competition peptides are added to the binding mixture, the amount of capture agents remaining bound to the immobilized peptide fragments will be accordingly reduced, depending on the amount/concentration of soluble competition peptides in the binding mixture. A standard curve for each specific target protein may be generated based on the amount of soluble competition peptides within the binding mixture, and the amount of capture agents remaining bound to the immobilized PET-containing peptide fragment on the array. Such a standard curve may be used to determine the amount of that target protein in an unknown sample. The method may also be used to simultaneously quantitate more than one target proteins within the sample, by generating a standard competition curve for each of the many target proteins. In this embodiment, the capture agents are usually labeled (e.g. fluorescent dye) for detection. The same label can be used for different capture agents in the same reaction if there is virtually no cross-reactivity.

In an alternative embodiment (the capture agent array), an array of capture agents are immobilized on a support. Each of the capture agents is specific for a given PET-containing peptide fragment within a sample. When such an array is in contact with a treated sample with the target PET-containing peptides of the capture agents, the PET-containing peptides will be bound by the capture agents. However, if a labeled competition PET-containing peptide is also present in the binding mixture, the labeled and unlabeled PET-containing peptides will compete for binding to the capture agent, in a concentration dependent manner. The amount of labeled PET-containing peptides bound to the immobilized capture agents will depend on the concentration of the competing unlabeled PET-containing peptides. Thus, a standard competition curve can be established by using a known concentration of labeled PET-containing peptide and a series of known concentrations of unlabeled PET-containing peptides. This standard curve can then be used to measure the concentration of the target PET-containing peptide in the sample. The method may also be used to simultaneously quantitate more than one target proteins within the sample, by generating a standard competition curve for each of the many target proteins. The same (or different) label can be used for different target peptides since their respective capture agents are located on distinct addressable locations on the support, and thus the same kind of signal can be readily distinguished by their locations on the support (array). In this embodiment, the peptides are usually labeled for detection.

When assessing expression profile of the same analytes in two (or more) different samples, it may be useful to obtain a quantitative readout for each protein that is being measured, as well as a differential assessment between protein levels between two samples. Gene chips have set the standard on differential measurement, where two different labels (typically fluorescent dyes) are incorporated into two different samples to be measured (each sample gets its own label). The relative gene expression between these two samples can be determined. In this way, one can compare, for example, “normal” samples with “disease” samples. For quantification of each gene, specific probes may be used to amplify and analyze the signal by quantitative PCR.

A similar approach may be adapted for differential protein assessment. The main advantages of the differential approach are: a) no need to provide a standard curve for each analyte; and, b) ability to handle a large dynamic range, as even abundant proteins, which on their own would saturate their antibodies and hence be out of range, are measurable when two samples are analyzed simultaneously. The amount of each differently labeled protein is below the saturation level of the antibody. The relative amount of each dye bound to the antibody reflects the amount of protein in the starting sample. In this way, one determines the relative expression of protein between one sample and another (e.g. two fold higher). The downside of the differential measurement is that there is no reliable way to compare results generated in different labs or between samples analyzed on different days, unless exactly the same reference sample is used and the sample needs to be labeled prior to analysis.

On the other hand, quantitative assays are routinely employed for immunoassays. In this type of assay, an assay standard is provided with the assay kit and a standard curve is generated as part of each measurement. The subject antibody design approach (e.g. the PET peptide antibodies) provides the level of selectivity needed to minimize antibody cross-talk when multiple types of antibodies are used in the same assay.

The two assay platforms described above (either peptide/small molecule array, or antibody array) both provide a quantification standard curve for each antibody/antigen (e.g. peptide or small molecule) pair. The standard curve may be constructed for all analytes (e.g. peptides) simultaneously, using several sample chambers on an array (e.g. a slide), while the remaining chambers can be used for different samples to be analyzed. Each chamber typically contains the same printing pattern of immobilized antigens or antibodies.

In certain embodiments, an improvement of the assay platforms combine aspects of both the differential and quantitative assay into one format, allowing capturing the benefits of both. For example, one labeling reagent may be used to label all the peptide standards (for example, using green dye for standard peptides 1, 2, and 3 to be measured). Meanwhile, a second, different labeling reagent (e.g. red dye) is used to label the sample to be measured. A mixture of the labeled peptide standards is provided in the assay kit at a known and predetermined concentration. The assay standard cocktail is combined with the labeled sample and applied to a single chamber that contains the immobilized antibody array. Each antibody in the chamber is consequently labeled with both dyes, where the quantity of the dyes reflects the relative amount of the analyte (e.g., peptide fragment containing the PET) between the peptide standard and the unknown sample. The data obtained may be reported in differential terms (e.g. “2 fold higher than standard” etc.) or in absolute terms (e.g. 0.01 mg/ml, etc.), since the concentration of each standard used is known. Since all results are calibrated to the standard provided, results can be compared across all measurements. This seeming straightforward approach is uniquely suited to the subject PET-based approach, since it is not practical to provide labeled whole proteins as standards due to complexities such as generating the whole proteins in the first place, and then keeping the labeled proteins stable. In addition, the total concentration of proteins in the labeled standard would be many folds higher (likely 10-100 fold higher) if whole proteins (instead of small PET-peptides) are used, practically limiting the number of standard peptides that may be included in the same reaction.

The benefits of this assay format include at least the following:
higher throughput—more chambers on each array/slide can be dedicated to samples, rather than being used to construct standard curves.
broader dynamic range—the low end of the detection range is determined by antibody affinity (k_{d}) and background relative to signal. The high end of the range is essentially infinite as long as the unknown sample and peptide standard can adequately compete for binding (e.g. one amount is not orders of magnitude greater than the other). User can adjust the concentration of the labeled peptide standard in their measurement to select the appropriate range for that sample. User can also adjust detector (e.g. PMT) settings to match the readout for each antibody within each sample chamber.
ability to accommodate chamber to chamber differences—it can be shown that the relative binding between two samples is insensitive to variability in antibody performance chamber to chamber, as any chamber-specific changes impact both the sample and the standard equally (the advantage of internal control). For the same reason, this assay format will be able to accommodate differences in antibody affinity between different lots of antibodies. Thus this assay represents a much more forgiving approach.

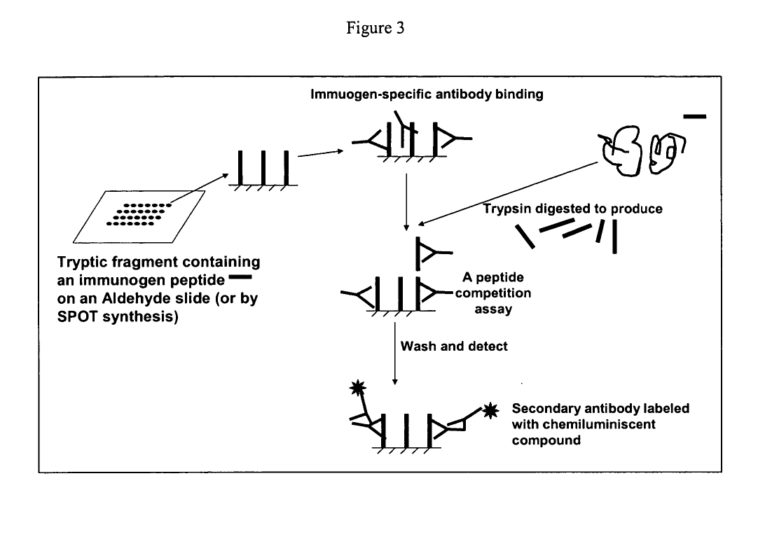

FIG. 3 illustrates an exemplary embodiment of the invention, in which the PET-containing peptides are immobilized on the array. In this illustrative example, the capture agents are antibodies specific for the immobilized PET-containing peptides. Instead of directly labeling the capture agent, a labeled secondary antibody specific for the capture agent is used for signal detection.

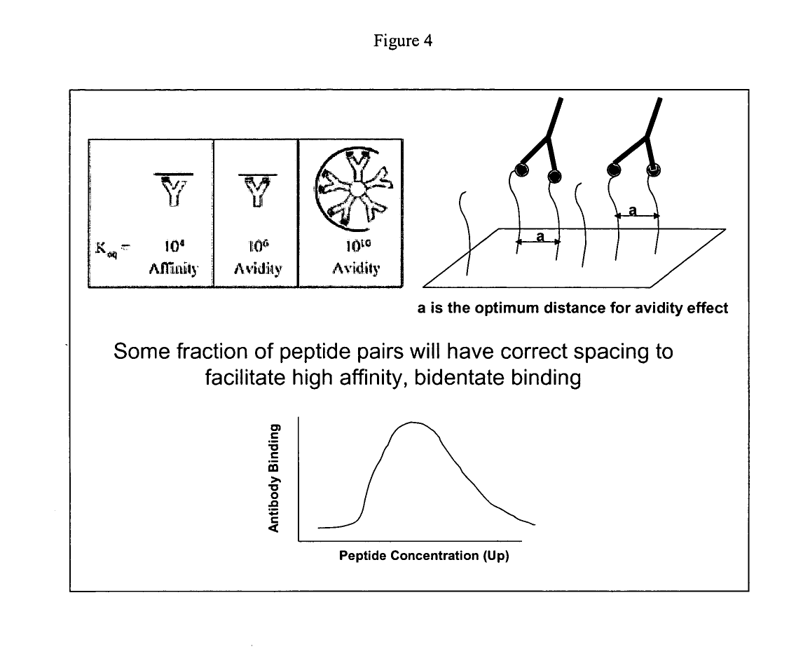

In general, as in the PET-based peptide array described in U.S. Ser. No. 60/519,530, such small molecule/peptide array is preferred embodiments over the alternative capture agent-based array, partly because of the several distinct advantages described below. First of all, immobilized analytes properly spaced on the support may facilitate high affinity, bidentate binding to certain capture agents, such as antibodies, resulting in overall enhanced avidity several magnitudes higher than the affinity between the normal antibody-antigen interaction. FIG. 4 is an illustrative example of this so-called “avidity effect.” The bottom panel shows that, even for the same antigen-antibody pair, as the concentration of the immobilized analyte increases, the apparent antibody binding affinity follows a bell-shaped curve. The apparent affinity first remains at a relatively low basal level (such as K_{eq}=10⁴), representing binding between a single antibody to a single antigen. As the antigen concentration increases, so does the apparent affinity, as more and more antibodies are now engaged in bidentate binding-assisted binding with higher avidity (K_{eq}=10⁶-10¹⁰). The apparent affinity then gradually returns to the basal level since higher density antigens on the support also tend to destroy the proper spacing critical for the high affinity bidentate binding. This illustrates that there is an optimum immobilized antigen concentration for each capture agent (such as antibody) used in the assay, depending on the structural features of the capture antibody and the nature (binding orientation, affinity, etc.) of the antibody-antigen interaction. If the immobilized analyte/antigen is of proper concentration, a relatively low affinity antibody with 100-1000 nM affinity may be transformed into a high affinity one with pico- or very low nano-molar range affinity antibody. An added advantage of this high affinity bidentate binding is that the antibody-antigen pair, now engaged in bidentate binding, might have a much longer half-life. It is estimated that half-lives of these immobilized peptide-antibody complexes are several hours or more, as compared to those of the same pairs measured in solution (usually about 10 seconds). This is an increase of about 2-3 orders of magnitude in half-life (see Naffin et al., Chem Biol. 10(3): 251-9, 2003, reporting that high-affinity bidentate capture agents for dimeric proteins can be created by simply immobilizing modest-affinity ligands on a surface at high density).

For PET-based peptide arrays, there is an additional advantage in that the subject PET peptide arrays use short PET sequences in the arrays, while the capture agents arrays use relatively large antibody molecules if the capture agents are antibodies. The short PET peptides are almost always more stable than the large antibody molecules on solid supports, giving the PET peptide arrays longer shelf life and better stability.

In certain embodiments, capture agents can be antibodies, or any other suitable capture agents described below.

In yet other related embodiments, the invention provides arrays of small molecules and/or PET-based peptides in similar competition assays.

Another aspect of the invention provides methods and reagents for a high throughput assay development platform, which can be used, for example, in large scale (genome-wide or metabolome-wide) screening of analyte concentration changes in a sample, which can be used to identify biomarkers as surrogate end points for diagnosis, monitoring treatment, and/or prognosis.

For example, small molecule metabolites and proteins found in human plasma perform many important functions in the body, and over or under expression/presence of these metabolites/proteins can either cause disease directly, or reveal its presence (disease marker). It is entirely foreseeable that many, if not most diseases, will more or less affect the level of at least one serum protein or metabolites in a diseased individual. This makes serum an attractive sample source for disease diagnosis and treatment monitoring. Thus it is not surprising that over $1 billion annually is spent on immunoassays to measure proteins in plasma as indicators of disease (Plasma Proteome Institute (PPI), Washington, D.C.).

Numerous immunoassays have also been developed for various small molecules as disease or environmental markers (see commercial kits from EnviroLogix, Portland, Me.). Metabolic profiles of bodily fluids such as plasma, cerebrospinal fluid and urine reflect both normal variation and the physiological impact of disease and pharmaceuticals on organ systems. Hundreds to thousands of low-molecular-weight metabolites in these body fluids collected from healthy and diseased populations have been tracked and quantified.

However, despite decades of research, only a handful of proteins (about 20) among the 500 or so detected proteins in plasma are measured routinely for diagnostic purposes. One of the major obstacles in developing more serum markers for diagnosis/monitoring of various diseases is the lack of large scale screening means to detect/quantitate/profile serum metabolite/protein levels or changes thereof in normal or diseased samples.

Part of the reason is that proteins and metabolites in plasma differ in concentration by at least one billion-fold. For example, serum albumin has a normal concentration range of 35-50 mg/mL (35-50×10⁹ pg/mL) and is measured clinically as an indication of severe liver disease or malnutrition, while interleukin 6 (IL-6) has a normal range of just 0-5 pg/mL, and is measured as a sensitive indicator of inflammation or infection. Another reason is that antibodies against different antigens, especially specific epitopes of specific proteins, tend to have a wide range of affinities for their antigens. The combination of these two common problems rendered it very difficult to produce a large scale screening methods that can simultaneously detect/profile different serum proteins/metabolites in the same sample.

To illustrate, if antibody 1 has a high affinity for antigen A, while antibody 2 has a low affinity for antigen B, assuming antigens A and B both have similar concentrations in a sample, binding of antibody 1 to antigen A may be already saturated before binding of antibody 2 to antigen B has even reached a detectable level. This so-called “dynamic range” problem may be even worse when there is higher level of antigen A than antigen B in the sample. In another scenario, if both antibodies 1 and 2 have similar affinities, while antigens A and B have vastly different concentrations in the sample (as is usually the case for two serum proteins), the same dynamic range problem will result. This problem is not unique to antibody-antigen binding, but generally exists between different pairs of capture agent/binding partner interaction.

One way to correct this problem is to adjust the amount of antibodies/capture agents with vastly different affinities, and/or the amount of immobilized antigens (PET peptides and/or small molecules) on the support, taking account the normal levels of their respective analytes (PET-containing antigens and/or small molecules) in a sample. If properly adjusted, all antigen-antibody reactions will be expected to generate similar amount of binding (detectable signals), making it possible to simultaneously detect the concentration changes, if any, in a large number of analyte targets within a sample. This type of adjustment can be routinely done using the simple equation (A+B [symbol] AB) for measuring binding affinity (K_{d}), the known affinity (K_{d}) of any capture agent in question, and the rough amount of the particular analyte in the sample.

Thus the instant invention provides a high throughput assay development platform for designing and manufacturing small molecule and/or PET-based peptide arrays, which can be used in simultaneous detection/quantitation of concentration changes, if any, in a large number of analyte targets within a sample.

In PET-peptide arrays for plurality of protein targets with a wide range of concentrations within a sample, PET sequences of these target proteins can be identified using a variety of knowledge databases of the instant application. These include (but are not limited to): PET relation database, which ranks proteome-wise PET uniqueness based on the number and quality of its nearest neighbors; PET antigenicity database (ranks or assigns absolute or relative values for antigenicity for each PET); protein cleavage database (information about proteome-wise peptide fragments after certain protease digestion or chemical treatment); PET conservation database (cross-species changes in PET); PET modification database (modifications associated with PET sequences or PET-containing peptide fragments), etc. Once the PETs are identified for each of these target proteins, capture agents, such as capture antibodies are raised against the PET sequences.

On the other hand, capture agents for small molecules can be obtained using the methods described below (see, for example, “antibody” section in the “Type of capture agents”).

Capture agent (e.g. Ab) cross-reactivity and affinity can be readily assessed for each capture agent/analyte pair (e.g. PET Ab-PET pair). Based on the affinity and specificity of a particular capture agent-analyte pair, and the normal amount of the corresponding target analytes within the sample, the amount of each immobilized antigen can be adjusted, such that when it is immobilized on a support, roughly the same amount of antibody binding to the immobilized analyte (and thus detectable signal) can be anticipated. In the serum disease marker screening scenario, this type of “normalized” array can be used for large scale screening of potential disease markers, since in a normal serum sample, all signals are expected to be within the same signal detection range. If a particular disease significantly affects the level of a given set of serum proteins or metabolites, signals corresponding to these proteins or metabolites will be easily detected/quantitated. The method can be further improved by using several dilutions of a test sample, such that analytes present in high concentration, although initially outside the dynamic range of detection, may be brought into the effective detection range in one of the diluted samples.

This method is particularly useful when the affinities of various capture agents are distributed over a wide range, such that the affinity of the highest affinity capture agents are at least 2, 3, 4, 5, 6, or more magnitudes higher than those of the lowest affinity capture agents. The method is also particularly useful when the normal concentrations of the plurality of target analytes in a sample are distributed over a wide range, such that the concentration of the highest concentration target analytes are at least 2, 3, 4, 5, 6, 7, 8, 9, 10, or more magnitudes higher than those of the lowest concentration target analytes.

A further useful product of the instant invention is a metabolite knowledge database derived from data obtained using the various embodiments of the instant invention. Such database may include information such as normal ranges of certain metabolites in certain tissues or samples, effects of various agents (such as drugs) on such ranges (including changes over time), established surrogate markers associated with certain disease or conditions, etc. The database may also has linkages to protein and gene expression databases, such that a new and fundamental understanding of organismic responses to environmental insult may emerge from the integration of metabonomic data with those obtained from the study of global patterns of gene and protein expression.

For example, the invention relates to a series of PET knowledge databases, including (but not limited to): PET epitope affinity database; PET epitope cross-reactivity database; and PET epitope assay parameter database. As more and more PET sequences are used for capture agents generation, accumulative knowledge about the association among PET sequences, PET antibody quality (binding affinity, specificity, etc.), and the performance of specific PET antibodies in specific assay formats are not only valuable information on their own rights, but also supplements the original databases on which the PET sequences are designed. Based on these databases, it would be possible to understand and eventually predict whether a particular PET sequences, based on its sequence content and context, tend to generate high/low affinity and/or specificity antibodies.

These methods are generally more suitable for immobilized small peptides, rather than large, native proteins. For one thing, it is much easier to achieve relatively uniform orientation of the immobilized PET-peptides on the support, so that bidentate binding is easier to occur. While for native proteins, it is conceivably more difficult to have these proteins to orientate in a similarly orderly fashion. Furthermore, large proteins are more prone to denaturation on solid support, thus arrays of native proteins tend to have much shorter half-lives for practical uses. And finally, the PET sequences are especially suitable for this type of array, since nearest neighbor peptides may be included for a better definition of antibody cross-reactivity.

Sample to be assayed is optionally fragmented, denatured (chemical or thermal, see U.S. Ser. No. 60/519,530) or solubilized (using detergent-based or detergent free, i.e., sonication, methods) to reduce their complexity. The sample as used herein includes any body sample such as blood (serum or plasma), sputum, ascites fluids, pleural effusions, urine, biopsy specimens, isolated cells and/or cell membrane preparation. Methods of obtaining tissue biopsies and body fluids from mammals are well known in the art. The instant methods may also be used in quantitating analytes in other non-biological samples, such as environmental samples.

For example, retrieved biological samples can be further solubilized using detergent-based or detergent free (i.e., sonication) methods, depending on the biological specimen and the nature of the examined polypeptide (i.e., secreted, membrane anchored or intracellular soluble polypeptide).

In certain embodiment, the sample may be denatured by detergent-free methods, such as thermo-denaturation. This is especially useful in applications where detergent needs to be removed or is preferably removed in future analysis.

In certain embodiments, the solubilized biological sample is contacted with one or more proteolytic agents. Digestion is effected under effective conditions and for a period of time sufficient to ensure complete digestion of the diagnosed polypeptide(s). Agents that are capable of digesting a biological sample under moderate conditions in terms of temperature and buffer stringency are preferred. Measures are taken not to allow non-specific sample digestion, thus the quantity of the digesting agent, reaction mixture conditions (i.e., salinity and acidity), digestion time and temperature are carefully selected. At the end of incubation time proteolytic activity is terminated to avoid non-specific proteolytic activity, which may evolve from elongated digestion period, and to avoid further proteolysis of other peptide-based molecules (i.e., protein-derived capture agents), which are added to the mixture in following steps.

If the sample is thermo-denatured, protease active at high temperatures, such as those isolated from thermophilic bacteria, can be used after the denaturation.

The present invention is based, at least in part, on the realization that PET can be identified by computational analysis, can characterize individual proteins in a given sample, e.g., identify a particular protein from amongst others. The use of agents that bind PETs can be exploitated for the detection and quantitation of individual proteins from a milieu of several or many proteins in a (biological) sample. The subject method can be used to assess the status of proteins or protein modifications in, for example, bodily fluids, cell or tissue samples, cell lysates, cell membranes, etc. In certain embodiments, the method utilizes a set of capture agents which discriminate between splice variants, allelic variants and/or point mutations (e.g., altered amino acid sequences arising from single nucleotide polymorphisms).

As a result of the sample preparation, namely denaturation and/or proteolysis, the subject method can be used to detect specific proteins/modifications in a manner that does not require the homogeneity of the target protein for analysis and is relatively refractory to small but otherwise significant differences between samples. The methods of the invention are suitable for the detection of all or any selected subset of all proteins in a sample, including cell membrane bound and organelle membrane bound proteins.

Another aspect of the invention provides a method of screening for potential marker(s) associated with certain conditions, especially those biomarker(s) that are potentially surrogate endpoints for clinical uses. In certain embodiments, a large panel of small molecules or PET-containing proteins of interest can be selected and immobilized in an array format. Using the subject competition assay, these arrays of small molecules (and/or PET-peptides) can be used to measure/profile the levels of these candidate small molecules in certain test samples as compared to their respective control samples, so as to identify any markers that consistently and/or significantly exhibit changed levels in test vs. control samples.

To illustrate, metabolites and proteins with a sample (e.g. serum) may be identified using any of the art-recognized methods, including but are not limited to: NMR, Mass Spectrometry (MS), HPLC, LC/GC, 2-D gel, etc. One or more capture agents (e.g. antibodies) may be generated to each of these small molecules and epitopes of the proteins, using any of the subject method. These capture agents may be pre-screened using, for example, the proteome matrix chips or nearest neighbour peptides to select for ones with high specificity. These metabolites/peptides, and their specific capture agents can then be used to construct peptide or antibody arrays for use in various methods of the invention. An array with all the serum metabolites and serum proteins could be a valuable tool for expression profile studies, biomarker identification, and any other system biology studies.

This general method can be used to identify any marker or panels of markers associated with a specific condition. For example, the subject competition assay can be used to ascertain which, if any, of a panel of interested analytes may have changed levels in disease v. normal tissue, polluted v. clean environmental sample, or diseased tissues before and after treatment. Those analytes that have consistently and/or significantly changed levels in samples with the condition (e.g., diseased tissue, polluted sample, treated sample, etc.), as compared to samples without the condition (e.g., normal tissue, clean/unpolluted sample, untreated sample, etc.), are identified as markers associated with the condition.

“Significantly” changed refers to a substantial change, especially those changes that are consistently seen across the same type of sample from different individuals (individuals with similar/same disease, similarly polluted sample, patients in the same treatment group, etc.). In certain embodiments, “significantly changed” means, on average, a 5%, 10%, 20%, 50%, 100%, 2-fold, 5-fold, 10-fold, 50-fold, 100-fold, or even 1000-fold increase or decrease as compared to its control level. However, such significant change may not necessarily be statistically significant. Obviously, markers with statistically significant changes would be preferred. However, under certain circumstances, where there is no individual statistically significant markers, the use of a panel of less-than-ideal markers, such as those with significant change, but not statistically significant ones, may still be a preferable choice (or a more accurate measure) over a single marker.

The methods and reagents of the instant invention have wide applications in a number of fields, including: research and development in academic and industrial settings, medicine (predictive, preventive and personalized medicine, disease diagnosis—biomarker identification and measurement, etc.); pharmaceutical business (drug screening and development); natural and work environmental monitoring and protection; toxic substance control; food and cosmetic industry.

II. Definition

The following section provides definitions for certain terms used in the instant specification.

“Affinity” is the strength of binding between two molecules. In the antibody-antigen setting, affinity is the strength of binding between a single antigenic determinant and a single combining site on the antibody. It is the equilibrium constant that describes the Ag-Ab reaction (Ag+Ab→Ag-Ab, K_{eq}=[Ab-Ab]/([Ab][Ag])). The same equation can be used to broadly describe the binding strength between any two molecules, such as a small molecule metabolite and its binding partner (which can be an antibody or a specific protein).

“Avidity” when used in the antigen-antibody setting, is a measure of the overall strength of binding o an antigen with many antigenic determinants and multivalent antibodies (see FIG. 1, top left panel).

As used herein, the term “Proteome Epitope Tag,” or “PET” is intended to mean an amino acid sequence that, when detected in a particular sample, unambiguously indicates that the protein from which it was derived is present in the sample. See U.S. Ser. No. 60/519,530. For instance, a PET is selected such that its presence in a sample, as indicated by detection of an authentic binding event with a capture agent designed to selectively bind with the sequence, necessarily means that the protein which comprises the sequence is present in the sample. A useful PET must present a binding surface that is solvent accessible when a protein mixture is denatured and/or fragmented, and must bind with significant specificity to a selected capture agent with minimal cross reactivity. A unique recognition sequence is present within the protein from which it is derived and in no other protein that may be present in the sample, cell type, or species under investigation. Moreover, a PET will preferably not have any closely related sequence, such as determined by a nearest neighbor analysis, among the other proteins that may be present in the sample. A PET can be derived from a surface region of a protein, buried regions, splice junctions, or post translationally modified regions. An ideal PET is a peptide sequence which is present in only one protein in the proteome of a species. But a peptide comprising a PET useful in a human sample may in fact be present within the structure of proteins of other organisms. A PET useful in an adult cell sample is “unique” to that sample even though it may be present in the structure of other different proteins of the same organism at other times in its life, such as during embryology, or is present in other tissues or cell types different from the sample under investigation. A PET may be unique even though the same amino acid sequence is present in the sample from a different protein provided one or more of its amino acids are derivatized, and a binder can be developed which resolves the peptides.

When referring herein to “uniqueness” with respect to a PET, the reference is always made in relation to the foregoing. Thus, within the human genome, a PET may be an amino acid sequence that is truly unique to the protein from which it is derived. Alternatively, it may be unique just to the sample from which it is derived, but the same amino acid sequence may be present in, for example, the murine genome. Likewise, when referring to a sample which may contain proteins from multiple different organism, uniqueness refers to the ability to unambiguously identify and discriminate between proteins from the different organisms, such as being from a host or from a pathogen.

Thus, a PET may be present within more than one protein in the species, provided it is unique to the sample from which it is derived. For example, a PET may be an amino acid sequence that is unique to: a certain cell type, e.g., a liver, brain, heart, kidney or muscle cell; a certain biological sample, e.g., a plasma, urine, amniotic fluid, genital fluid, marrow, spinal fluid, or pericardial fluid sample; a certain biological pathway, e.g., a G-protein coupled receptor signaling pathway or a tumor necrosis factor (TNF) signaling pathway.

In this sense, the instant invention provides a method to identify application-specific PETs, depending on the type of proteins present in a given sample. This information may be readily obtained from a variety of sources. For example, when the whole genome of an organism is concerned, the sequenced genome provides each and every protein sequences that can be encoded by this genome, sometimes even including hypothetical proteins. This “virtually translated proteome” obtained from the sequenced genome is expected to be the most comprehensive in terms of representing all proteins in the sample. Alternatively, the type of transcribed mRNA species within a sample may also provide useful information as to what type of proteins may be present within the sample. The mRNA species present may be identified by DNA microarrays, SNP analysis, or any other suitable RNA analysis tools available in the art of molecular biology. An added advantage of RNA analysis is that it may also provide information such as alternative splicing and mutations. Finally, direct protein analysis using techniques such as mass spectrometry may help to identify the presence of specific post-translation modifications and mutations, which may aid the design of specific PETs for specific applications.

The PET may be found in the native protein from which it is derived as a contiguous or as a non-contiguous amino acid sequence. It typically will comprise a portion of the sequence of a larger peptide or protein, recognizable by a capture agent either on the surface of an intact or partially degraded or digested protein, or on a fragment of the protein produced by a predetermined fragmentation protocol. The PET may be 5, 6, 7, 8, 9, 10, 11, 12, 13, 14, 15, 16, 17, 18, 19 or 20 amino acid residues in length. In a preferred embodiment, the PET is 6, 7, 8, 9 or 10 amino acid residues, preferably 8 amino acids in length.

The term “discriminate”, as in “capture agents able to discriminate between”, refers to a relative difference in the binding of a capture agent to its intended protein analyte and background binding to other proteins (or compounds) present in the sample. In particular, a capture agent can discriminate between two different species of proteins (or species of modifications) if the difference in binding constants is such that a statistically significant difference in binding is produced under the assay protocols and detection sensitivities. In preferred embodiments, the capture agent will have a discriminating index (D.I.) of at least 0.5, and even more preferably at least 0.1, 0.001, or even 0.0001, wherein D.I. is defined as K_{d}(a)/K_{d}(b), K_{d}(a) being the dissociation constant for the intended analyte, K_{d}(b) is the dissociation constant for any other protein (or modified form as the case may be) present in sample.

As used herein, the term “capture agent” includes any agent which is capable of binding to a target analyte, such as a small molecule compound, a metabolite, or a protein that includes a PET sequence, e.g., with at least detectable selectivity. A capture agent is capable of specifically interacting with (directly or indirectly), or binding to (directly or indirectly) such an analyte. The capture agent is preferably able to produce a signal that may be detected. In a preferred embodiment, the capture agent is an antibody or a fragment thereof, such as a single chain antibody, or a peptide selected from a displayed library. In other embodiments, the capture agent may be a protein (natural or engineered), an RNA or DNA aptamer, an allosteric ribozyme or a small molecule. In other embodiments, the capture agent may allow for electronic (e.g., computer-based or information-based) recognition of a unique recognition sequence. In one embodiment, the capture agent is an agent that is not naturally found in a cell.

As used herein, the term “globally detecting” includes detecting at least 40% of the proteins in the sample. In a preferred embodiment, the term “globally detecting” includes detecting at least 50%, 60%, 65%, 70%, 75%, 80%, 85%, 90%, 95% or 100% of the proteins in the sample. Ranges intermediate to the above recited values, e.g., 50%-70% or 75%-95%, are also intended to be part of this invention. For example, ranges using a combination of any of the above recited values as upper and/or lower limits are intended to be included.

“Metabolites” are the end products of cellular regulatory processes, and their levels can be regarded as the ultimate response of biological systems to genetic or environmental agents including chemicals, drugs and nutritional factors.

“Metabolic profiling” involves measuring and interpreting complex, time-related, global changes in metabolites present in biological (or non-biological) samples, such as body fluids. The application of metabolic profiling technologies to biological systems is a powerful tool to study gene function in relation to disease (phenotype), predict toxicity of chemicals, drugs and nutritional agents in biological systems, identify markers of exposure and early disease status, and develop screening regimens for animal and human populations at increased risk of disease.

As used herein, the term “proteome” refers to the complete set of chemically distinct proteins found in an organism.

As used herein, the term “organism” includes any living organism including animals, e.g., avians, insects, mammals such as humans, mice, rats, monkeys, or rabbits; microorganisms such as bacteria, yeast, and fungi, e.g., Escherichia coli, Campylobacter, Listeria, Legionella, Staphylococcus, Streptococcus, Salmonella, Bordatella, Pneumococcus, Rhizobium, Chlamydia, Rickettsia, Streptomyces, Mycoplasma, Helicobacter pylori, Chlamydia pneumoniae, Coxiella burnetii, Bacillus Anthracis, and Neisseria; protozoa, e.g., Trypanosoma brucei; viruses, e.g., human immunodeficiency virus, rhinoviruses, rotavirus, influenza virus, Ebola virus, simian immunodeficiency virus, feline leukemia virus, respiratory syncytial virus, herpesvirus, pox virus, polio virus, parvoviruses, Kaposi's Sarcoma-Associated Herpesvirus (KSHV), adeno-associated virus (AAV), Sindbis virus, Lassa virus, West Nile virus, enteroviruses, such as 23 Coxsackie A viruses, 6 Coxsackie B viruses, and 28 echoviruses, Epstein-Barr virus, caliciviruses, astroviruses, and Norwalk virus; fungi, e.g., Rhizopus, neurospora, yeast, or puccinia; tapeworms, e.g., Echinococcus granulosus, E. multilocularis, E. vogeli and E. oligarthrus; and plants, e.g., Arabidopsis thaliana, rice, wheat, maize, tomato, alfalfa, oilseed rape, soybean, cotton, sunflower or canola.

As used herein, “sample” refers to anything which may contain an analyte suitable for the subject methods. The sample may be a biological sample, such as a biological fluid or a biological tissue. Examples of biological fluids include urine, blood, plasma, serum, saliva, semen, stool, sputum, cerebral spinal fluid, tears, mucus, amniotic fluid or the like. Biological tissues are aggregates of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s). The sample may also be a mixture of target protein containing molecules prepared in vitro.

“Small molecule” as used herein refers to molecules of any structure that has a molecular weight of less than about 5000 Dalton, preferably between about 50-3000, 50-2000, 50-1000, 50-500, or 50-200. It includes natural or synthetic compounds, metabolic intermediates, steroids, mono- or polysaccharides, lipids, pesticides, etc.

As used herein, “a comparable control sample” refers to a control sample that is only different in one or more defined aspects relative to a test sample, and the present methods, kits or arrays are used to identify the effects, if any, of these defined difference(s) between the test sample and the control sample, e.g., on the amounts and types of proteins expressed and/or on the protein modification profile. For example, the control bio-sample can be derived from physiological normal conditions and/or can be subjected to different physical, chemical, physiological or drug treatments, or can be derived from different biological stages, etc.

A report by MacBeath and Schreiber (Science 289 (2000), pp. 1760-1763) in 2000 established that proteins could be printed and assayed in a microarray format, and thereby had a large role in renewing the excitement for the prospect of a protein chip. Shortly after this, Snyder and co-workers reported the preparation of a protein chip comprising nearly 6000 yeast gene products and used this chip to identify new classes of calmodulin- and phospholipid-binding proteins (Zhu et al., Science 293 (2001), pp. 2101-2105). The proteins were generated by cloning the open reading frames and overproducing each of the proteins as glutathione-5-transferase-(GST) and His-tagged fusions. The fusions were used to facilitate the purification of each protein and the His-tagged family were also used in the immobilization of proteins. This and other references in the art established that microarrays containing thousands of proteins could be prepared and used to discover binding interactions. They also reported that proteins immobilized by way of the His tag—and therefore uniformly oriented at the surface—gave superior signals to proteins randomly attached to aldehyde surfaces.

Related work has addressed the construction of antibody arrays (de Wildt et al., Antibody arrays for high-throughput screening of antibody-antigen interactions. Nat. Biotechnol. 18 (2000), pp. 989-994; Haab, B. B. et al. (2001) Protein microarrays for highly parallel detection and quantitation of specific proteins and antibodies in complex solutions. Genome Biol. 2, RESEARCH0004.1-RESEARCH0004.13). Specifically, in an early landmark report, de Wildt and Tomlinson immobilized phage libraries presenting scFv antibody fragments on filter paper to select antibodies for specific antigens in complex mixtures (supra). The use of arrays for this purpose greatly increased the throughput when evaluating antibodies, allowing nearly 20,000 unique clones to be screened in one cycle. Brown and co-workers extended this concept to create molecularly defined arrays wherein antibodies were directly attached to aldehyde-modified glass. They printed 115 commercially available antibodies and analyzed their interactions with cognate antigens with semi-quantitative results (supra). Kingsmore and co-workers used an analogous approach to prepare arrays of antibodies recognizing 75 distinct cytokines and, using the rolling-circle amplification strategy (Lizardi et al., Mutation detection and single molecule counting using isothermal rolling circle amplification. Nat. Genet. 19 (1998), pp. 225-233), could measure cytokines at femtomolar concentrations (Schweitzer et al., Multiplexed protein profiling on microarrays by rolling-circle amplification. Nat. Biotechnol. 20 (2002), pp. 359-365).

Similarly, small molecule micro-arrays have been successfully used in a variety of setting including screening for drug targets. Kuruvilla et al. (Nature 416(6881): 653-7, 2002) demonstrate a potentially general and scalable method of identifying small molecules that bind to a particular protein. By probing a high-density microarray of immobilized small molecules generated by diversity-oriented synthesis with fluorescently labeled target protein, 3,780 protein-binding assays were performed in parallel, leading to the identification of several small molecule compounds that bind the target protein. These results demonstrate that diversity-oriented synthesis and small-molecule microarrays can be used to manufacture small molecule micro-arrays for various uses, such as identifying small molecules that bind to a protein of interest. The same method can also be used to immobilize selected small molecules/metabolites to generate micro-arrays containing these molecules for competition assay of the instant invention.

These examples demonstrate the many important roles that protein/small molecule microarray chips can play, and give evidence for the widespread activity in fabrication of these tools. The following subsections describes in further detail about various aspects of the invention.

III. Type of Capture Agents

In certain preferred embodiments, the capture agents used should be capable of selective affinity reactions with the target analyte (e.g., small molecules and PET moieties). Generally, such interaction will be non-covalent in nature, though the present invention also contemplates the use of capture reagents that become covalently linked to the analyte.

Examples of capture agents which can be used include, but are not limited to: nucleotides; nucleic acids including oligonucleotides, double stranded or single stranded nucleic acids (linear or circular), nucleic acid aptamers and ribozymes; PNA (peptide nucleic acids); proteins, including antibodies (such as monoclonal or recombinantly engineered antibodies or antibody fragments), T cell receptor and MHC complexes, lectins and scaffolded peptides; peptides; other naturally occurring polymers such as carbohydrates; artificial polymers, including plastibodies; small organic molecules such as drugs, metabolites and natural products; and the like.

In certain embodiments, the target analytes of interest are immobilized, permanently or reversibly, on a solid support such as a bead, chip, or slide. When employed to analyze a complex mixture of proteins and/or small molecules, the immobilized analytes are arrayed in addressable locations, and/or otherwise labeled for deconvolution of the binding data to yield identity of the analyte and to quantitate binding.

In one embodiment, the capture agents are conjugated with a reporter molecule such as a fluorescent molecule or an enzyme, and used to detect the quantity of capture agents remaining bound to the immobilized analytes on a support (such as a chip or bead). Alternatively, a secondary agent specific for the bound capture agent may be labeled to facilitate the detection and quantification of the bound capture agent.

An important advantage of the invention is that useful capture agents can be identified and/or synthesized even in the absence of a sample of the analyte to be detected, since the target metabolite or small molecule compound of interest is typically known and can be used to generate specific capture agents.

For instance, in the case of PET peptides, and with the completion of the whole genome in a number of organisms, such as human, fly (Drosophila melanogaster) and nematode (C. elegans), PET of a given length or combination thereof can be identified for any single given protein in a certain organism, and capture agents for any of these proteins of interest can then be made without ever cloning and expressing the full length protein.

In addition, the suitability of any PET to serve as an antigen or target of a capture agent can be further checked against other available information. For example, since amino acid sequence of many proteins can now be inferred from available genomic data, sequence from the structure of the proteins unique to the sample can be determined by computer aided searching, and the location of the peptide in the protein, and whether it will be accessible in the intact protein, can be determined. Once a suitable PET peptide is found, it can be synthesized using known techniques. With a sample of the PET in hand, an agent that interacts with the peptide such as an antibody or peptidic binder, can be raised against it or panned from a library. In this situation, care must be taken to assure that any chosen fragmentation protocol for the sample does not restrict the protein in a way that destroys or masks the PET. This can be determined theoretically and/or experimentally, and the process can be repeated until the selected PET is reliably retrieved by a capture agent(s).

The PET set selected according to the teachings of the present invention can be used to generate peptides either through enzymatic cleavage of the protein from which they were generated and selection of peptides, or preferably through peptide synthesis methods.

Proteolytically cleaved peptides can be separated by chromatographic or electrophoretic procedures and purified and renatured via well known prior art methods.

Synthetic peptides can be prepared by classical methods known in the art, for example, by using standard solid phase techniques. The standard methods include exclusive solid phase synthesis, partial solid phase synthesis methods, fragment condensation, classical solution synthesis, and even by recombinant DNA technology. See, e.g., Merrifield, J. Am. Chem. Soc., 85:2149 (1963), incorporated herein by reference. Solid phase peptide synthesis procedures are well known in the art and further described by John Morrow Stewart and Janis Dillaha Young, Solid Phase Peptide Syntheses (2nd Ed., Pierce Chemical Company, 1984).

Synthetic peptides can be purified by preparative high performance liquid chromatography [Creighton T. (1983) Proteins, structures and molecular principles. WH Freeman and Co. N.Y.] and the composition of which can be confirmed via amino acid sequencing.

In addition, other additives such as stabilizers, buffers, blockers and the like may also be provided with the capture agent.

A. Antibodies

In one embodiment, the capture agent is an antibody or an antibody-like molecule (collectively “antibody”). Thus an antibody useful as capture agent may be a full length antibody or a fragment thereof, which includes an “antigen-binding portion” of an antibody. The term “antigen-binding portion,” as used herein, refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see, e.g., Bird et al. (1988) Science 242:423-426; and Huston et al. (1988) Proc. Natl. Acad. Sci. USA 85:5879-5883; and Osbourn et al. 1998, Nature Biotechnology 16: 778). Such single chain antibodies are also intended to be encompassed within the term “antigen-binding portion” of an antibody. Any V_{H} and V_{L} sequences of specific scFv can be linked to human immunoglobulin constant region cDNA or genomic sequences, in order to generate expression vectors encoding complete IgG molecules or other isotypes. V_{H} and V_{L} can also be used in the generation of Fab, Fv or other fragments of immunoglobulins using either protein chemistry or recombinant DNA technology. Other forms of single chain antibodies, such as diabodies are also encompassed. Diabodies are bivalent, bispecific antibodies in which V_{H} and V_{L} domains are expressed on a single polypeptide chain, but using a linker that is too short to allow for pairing between the two domains on the same chain, thereby forcing the domains to pair with complementary domains of another chain and creating two antigen binding sites (see, e.g., Holliger, P., et al. (1993) Proc. Natl. Acad. Sci. USA 90:6444-6448; Poljak, R. J., et al. (1994) Structure 2:1121-1123).

Still further, an antibody or antigen-binding portion thereof may be part of a larger immunoadhesion molecule, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques.

Antibodies may be polyclonal or monoclonal. The terms “monoclonal antibodies” and “monoclonal antibody composition,” as used herein, refer to a population of antibody molecules that contain only one species of an antigen binding site capable of immunoreacting with a particular epitope of an antigen, whereas the term “polyclonal antibodies” and “polyclonal antibody composition” refer to a population of antibody molecules that contain multiple species of antigen binding sites capable of interacting with a particular antigen. A monoclonal antibody composition, typically displays a single binding affinity for a particular antigen with which it immunoreacts.

Any art-recognized methods can be used to generate an analyte-directed antibody. For example, a PET or a small molecule (alone or linked to a hapten) can be used to immunize a suitable subject, (e.g., rabbit, goat, mouse or other mammal or vertebrate). For example, the methods described in U.S. Pat. Nos. 5,422,110; 5,837,268; 5,708,155; 5,723,129; and 5,849,531 (the contents of each of which are incorporated herein by reference) can be used. The immunogenic preparation can further include an adjuvant, such as Freund's complete or incomplete adjuvant, or similar immunostimulatory agent. Immunization of a suitable subject with an antigen induces a polyclonal antibody response. The anti-analyte antibody titer in the immunized subject can be monitored over time by standard techniques, such as with an enzyme linked immunosorbent assay (ELISA) using immobilized analyte (e.g. PET).

Antibodies have been routinely raised against various small molecules such as pesticides/metabolites. For example, EnviroLogix (Portland, Me.) offers numerous commercial kits for detecting and quantitation various agents such as pesticides (Acetanilides, Alachlor, Alachlor mercapturate, Aldicarb, Atrazine, Atrazine mercapturate) and toxins (Aflatoxin), etc.

The antibody molecules directed against an analyte, such as a small molecule, can be isolated from the mammal (e.g., from the blood) and further purified by well known techniques, such as protein A chromatography to obtain the IgG fraction. At an appropriate time after immunization, e.g., when the anti-analyte antibody titers are highest, antibody-producing cells can be obtained from the subject and used to prepare, e.g., monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem 0.255:4980-83; Yeh et al. (1976) Proc. Natl. Acad. Sci. USA 76:2927-31; and Yeh et al. (1982) Int. J. Cancer 29:269-75), the more recent human B cell hybridoma technique (Kozbor et al. (1983) Immunol Today 4:72), or the EBV-hybridoma technique (Cole et al. (1985), Monoclonal Antibodies and Cancer Therapy, Alan R. Liss, Inc., pp. 77-96). The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lerner (1981) Yale J. Biol. Med., 54:387-402; M. L. Gefter et al. (1977) Somatic Cell Genet. 3:231-36). Briefly, an immortal cell line (typically a myeloma) is fused to lymphocytes (typically splenocytes) from a mammal immunized with an analyte immunogen as described above, and the culture supernatants of the resulting hybridoma cells are screened to identify a hybridoma producing a monoclonal antibody that binds the analyte.

Any of the many well known protocols used for fusing lymphocytes and immortalized cell lines can be applied for the purpose of generating a monoclonal antibody (see, e.g., G. Galfre et al. (1977) Nature 266:55052; Gefter et al. Somatic Cell Genet., cited supra; Lerner, Yale J. Biol. Med., cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NSI/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind an analyte, e.g., using a standard ELISA assay.

In addition, automated screening of antibody or scaffold libraries against arrays of target analytes will be the most rapid way of developing thousands of reagents that can be used for protein expression profiling. Furthermore, polyclonal antisera, hybridomas or selection from library systems may also be used to quickly generate the necessary capture agents. A high-throughput process for antibody isolation is described by Hayhurst and Georgiou in Curr Opin Chem Biol 5(6):683-9, December 2001 (incorporated by reference).

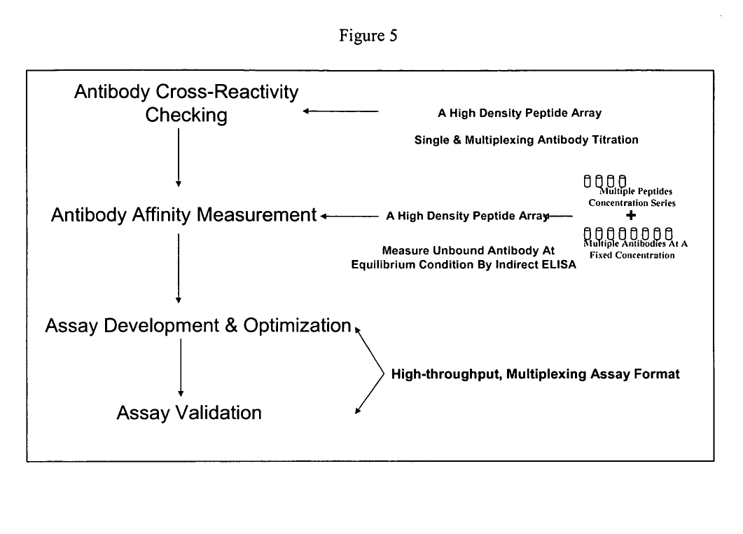

Once the candidate capture agent antibodies are generated, a high-throughput array-based antibody characterization and assay development platform may be used to efficiently identify the most useful antibodies for the purpose of the instant invention. FIG. 5 illustrates an exemplary embodiment of this assay development platform. Briefly, high-density peptide arrays may be employed to check antibody cross-reactivity, followed by antibody affinity measurement, to identify the most suitable antibodies with the highest affinity and the least cross-reactivity to a structurally similar antigen (e.g. the nearest neighbors of the PET peptides).

In certain embodiments, a “proteome matrix chip” may be used to facilitate proteome-wide testing of antibody specificity. As used herein, “proteome” does not necessarily mean a collection of all the proteins encoded by an organism's genome. Rather, it refers to a specific collection of all proteins within a given sample (e.g. a body fluid such as serum, a tissue, an organ, or an organism, etc.), or a part thereof (e.g. the top 100, 500, or 1000 most abundant protein of the sample; the phosphorylated subset, etc.). As used herein, “proteome matrix chip” refers to a peptide array representing all proteins/peptide fragments of the selected proteome, or a selected collection of such peptides. For example, In certain embodiments, a human proteome matrix chip may include all known human protein that can be encoded by the human genome. In certain embodiments, it may include all tryptic fragments of all known human proteins. In certain embodiments, it may include the top 100, 300, 500, or 1000 most abundant human proteins (or all their tryptic fragments). In certain embodiments, all theoretically possible peptides of a given length may be synthesized and tested. For example, to test all 6-mer peptides, 206 peptides may be individually synthesized for use in the subject arrays. In a related embodiment, a more selective theoretical approach may be used to reduce the amount of peptides that needs to be tested. For example, for a 6-mer peptide, one or two (or any other number of) especially important residue(s) may be fixed, while all other positions are allowed to be substituted by any of the 20 naturally occurring amino acids. In certain embodiments, any of the above-described collections of peptides may exclude certain peptides not suitable for array detection, such as highly hydrophobic or highly “sticky” peptides that tend to bind nonspecifically to a large number of other molecules.

Any of the art-recognized methods may be used to determine the identity and abundance of each expressed protein. For example, mass spectrometry, 2-D gel analysis, literature search, mRNA expression data, etc., or combinations thereof.

In certain embodiments, the selected peptides are synthesized by, for example, polypeptide synthesizer (such as solid phase synthesis utilizing the FMOC chemistry and an automated Applied Biosystems 432A peptide synthesizer). In certain embodiments, the selected peptides are recombinantly produced. In certain embodiments, the selected peptides are biochemically purified and is substantially free of contaminants (e.g. at least about 95% pure, or 99% pure, etc.). In certain embodiments, at least certain proteins, especially any small proteins with less than about 200 residues may be used directly without digestion. In certain embodiments, most or all proteins are represented as polypeptides (not full-length proteins) on the proteome matrix chip/array, preferably tryptic fragments. In certain embodiments, at least one protein in the proteome is represented by more than one peptide fragments from the protein, preferably non-overlapping fragments.

Such chips/arrays are particularly useful to comprehensively assess the cross-reactivity (and thus specificity) of any given capture agents (e.g. antibodies), since such tests are conducted on a proteome-wide scale. Using such proteome matrix chip, capture agents identified using any of the subject methods may be screened against the proteome in which they are intended to be used (e.g. all serum proteins). Since two capture agents directed to different fragments/epitopes of the same protein are unlikely to recongnize the same set of cross-reacting peptides, overall assay accuracy may be considerably improved by using two or more capture agents against different epitopes of the same target analyte.

In certain embodiments, the amount of immobilized individual peptides on the proteome matrix chip/array may be adjusted to reflect the relative abundance of these peptides under physiological conditions. For example, if serum proteins 1 and 2 are normally present in serum at a 2:1 ratio, twice amount of protein 1 peptides may be spotted than that of protein 2 peptides in the chip. This adjustment might be advantageous, since a relatively low cross-reacting antibody may exibit significant non-specific binding at the presence of relatively large amounts of non-specific peptides.

To illustrate, in an exemplary embodiment, ˜1000 discovered serum proteins may be identified as the proteome in question. Predicted tryptic peptides from, for example, the top 100, 300, 500, or 1000 (all) most abundant serum proteins will then be generated (e.g. in sillico). All or a part of these peptide fragments may be used in a peptide chip for specificity/cross-reactivity test for capture agents (e.g. antibodies). The level of each peptide may be “normalized” according to their relative serum concentration, such that high concentration proteins may be realistically represented in the array/chip by a spot of higher peptide concentration.

Thus in certain embodiments, antibodies are screened against proteome matrix chip peptides, which are present on the chip at their respective expected concentrations in the sample of interest. Such arrangement demonstrates an appropriate level of specificity for the desired measurement. Alternatively, in certain other embodiments, all peptides on the proteome matrix chip have the same concentration. Antibody affinity for cognate target antigen, relative to cross-reactive peptides can then be estimated through titration.

To facilitate quantitative comparison of capture agent (e.g. antibody) specificity and cross-reactivity, a key parameter “KC” for each tested antibody against each antigen, defined as “Ab binding constant (K)×peptide concentration (C)” can be used. For example, in a binding reaction between Ab and its ligand L:
[Ab]+[L]==[Ab−L]
K_{L}=[Ab−L]/([Ab]*[L]) (the greater the value of K_{L}, the tighter the bidning between Ab and L)

Similarly, for each potential cross-reacting peptides C1, C2, C3, etc:
[Ab]+[Ci]=[Ab−Ci] (i=1, 2, 3, etc.)
K_{C1}=[Ab−C1]/([Ab]*[C1])
K_{C2}=[Ab−C2]/([Ab]*[C2])
K_{C3}=[Ab−C3]/([Ab]*[C3])

Specificity S can be defined as:
(K_{C1}*[C1]+K_{C2}*[C2]+ . . . +K_{Cn}*[Cn])/(K_{L}*[L])

Where there are “n” cross-reacting polypeptides. Thus, Specificity S can be viewed as the likelihood of a particular antibody, at the specific test condition, to bind cross-reacting peptides as opposed to bind its cognate peptide. A high specificity Ab is expected to have a specificity value S of close to 0 (only negligible amount bound to all cross-reacting peptides combined), while larger specificity values indicate poor selectivity towards its cognate peptides.

In certain embodiments, the specificity value S for a selected Ab is no more than about 0.2, preferably no more than about 0.1, about 0.05, about 0.02, about 0.01, or about 0.001 or less. Most preferably, 0 within the detection limit.

In certain embodiments, at least about 30%, 40%, 50%, 60%, 70%, 80%, 90%, 95%, 99%, or substantially all of the capture agents used in the subject mathods have specificity value S of no more than about 0.1, preferably no more than about 0.05, 0.02, or 0.01.

Data obtained from such specificity tests could be used either: a) to screen out/discard antibodies with unacceptable properties that are undesirable for use in a particular product, and/or b) to provide directions for individual users on reliability and limitation of some or all selected antibodies.

In certain embodiments, not all tryptic fragments are used on the proteome matrix chip. Instead, certain parameters can be employed to select specific (tryptic) peptide fragments for use on the peptide array. These may include: eliminating obviously hydrophobic peptides to eliminate non-specific binding; consider length and other parameters for final peptide selection.

Once these appropriate peptide fragments are selected, a peptide array for antibody cross-reactivity screening may be made, for example, by spotting 10 pg of each of these peptide fragments on a peptide array. Each capture agent (e.g. Ab) will then be applied (for exmaple, at a concentration of about 1 nM) to these peptides to screen for any non-specific cross reactiviy.

If a certain peptide (e.g. tryptic fragment) is found to cross-react with a particular capture agent, the effect of that cross-reacting peptide on the binding of cognate PET may be further assessed. For example, the capture agent (e.g. Ab) may be spotted as a series of spots at an amount of about 100 pg/spot. A dose-response curve of the labeled cognate PET may be established, at the presence of physiological concentration of the cross-reacting peptides identified above. Such screening would provide the best available capture agents with the right combination of affinity and specificity, with user being aware of the reliability and limitation of any obtained data.

Antibody capture agents identified through these assays are then used for individual assay development, optimization, and validation.

For example, for antibody cross-reactivity verification, in a slide of 16 chambers, each chamber may has, for example, about 100 distinct analytes. Each one chamber can then be used to verify the cross-reactivity of one candidate antibody. If the antibody only reacts with one but not other 99 analytes in the same chamber, then it is deemed specific. To verify all 100 antibodies, approximately 6 parallel assays (6×16=96) have to be run. In a preferred embodiment, the total number of immobilized analytes in each chamber may be reduced so that each analyte may have several different printed concentrations. In addition, for each target analyte immobilized on the slide, one or more structurally related compounds, such as the nearest neighbor peptides, may also be included as negative controls.

For antibody affinity measurement, the same slide construction may be used. However, each chamber can in theory be used for simultaneous measurement for all the antibodies, if it can be assumed that binding of one antibody does not interfere with the binding of a different antibody, and that the overall concentration of all antibodies in solution is not too high. For example, assuming 100 immobilized analytes (of known concentration) are present in each chamber, a solution of 100 antibodies can be added to one chamber. Each antibody is about 1 pM to 1 μM in concentration (total 100 pM-100 μM). By measuring the bound antibodies at discrete locations, the K_{d}'s for each of the 100 antibodies can be readily calculated by using data from a single well, including the total amount of each immobilized analyte on each spot, the amount of bound antibodies at equilibrium. Certainly, less than 100 antibodies can be added to each chamber if the overall concentration of antibodies is a concern. In a related embodiment, different concentrations of the same set of 100 analytes can be printed in the other 15 chambers. If the same antibody cocktail is used for each chamber to measure K_{d}'s, due to the bidentate binding effect, an optimal printed concentration for each analyte can be determined for each antibody-antigen pair. Antibody cross-reactivity can also be checked using a similar assay format.

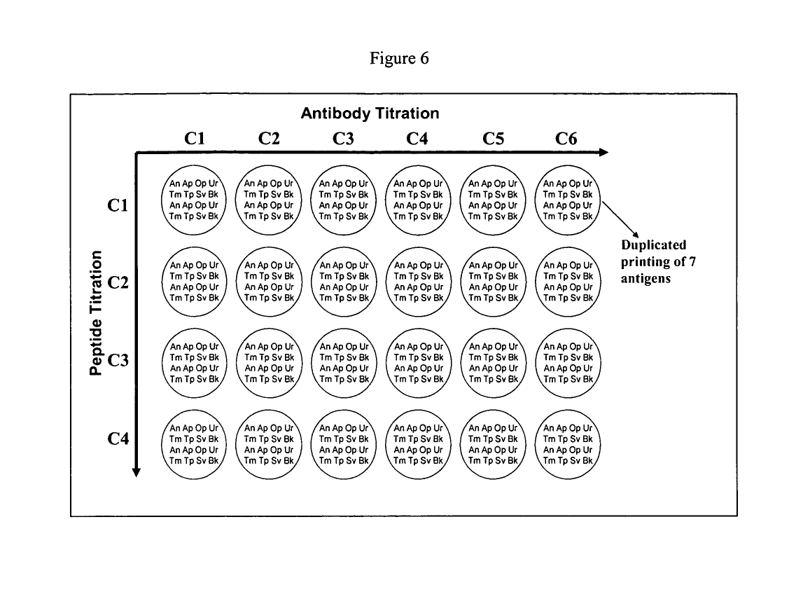

FIG. 6 is an illustrative example of the high-density peptide arrays for multiplexing antibody and peptide titration. For each well, 7 antigens (plus one blank control) are immobilized on the support by duplicated printing. For part of the whole plate shown, each wells in a row can be used for antibody titration using different antibody concentrations, while each wells in a column contains a different concentration of target peptides.

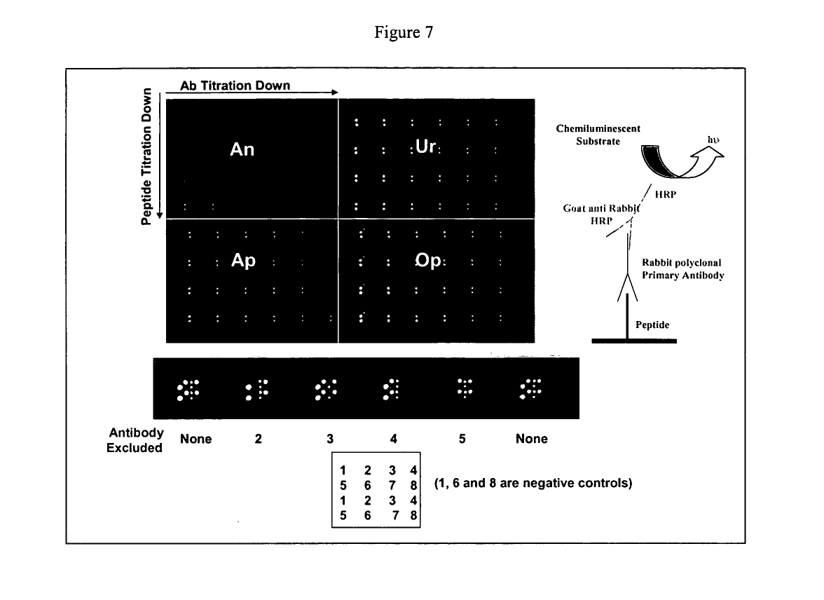

FIG. 7 is an exemplary result obtained from one of the assays. The right side illustrates the format of the peptide competition assay with immobilized PET peptides. An HRP-conjugated Goat anti-rabbit secondary antibody is used for the ELC reaction to detect the amount of bound rabbit polyclonal primary antibodies. The top 4 panels on the left side show the results of titrating down both the antibody and the competitor antigen for An, Ur, Ap and Op. For each of the four proteins, four concentrations of competitor peptides are used for each antibody titration curve. The middle and bottom panels show the relative specificity of the antibodies. When specific antibodies are excluded from the assay mixture, ECL signals corresponding to the respective antigens are also missing, demonstrating that the other antibodies do not react with the immobilized PETs except for their respective antigen PETs.

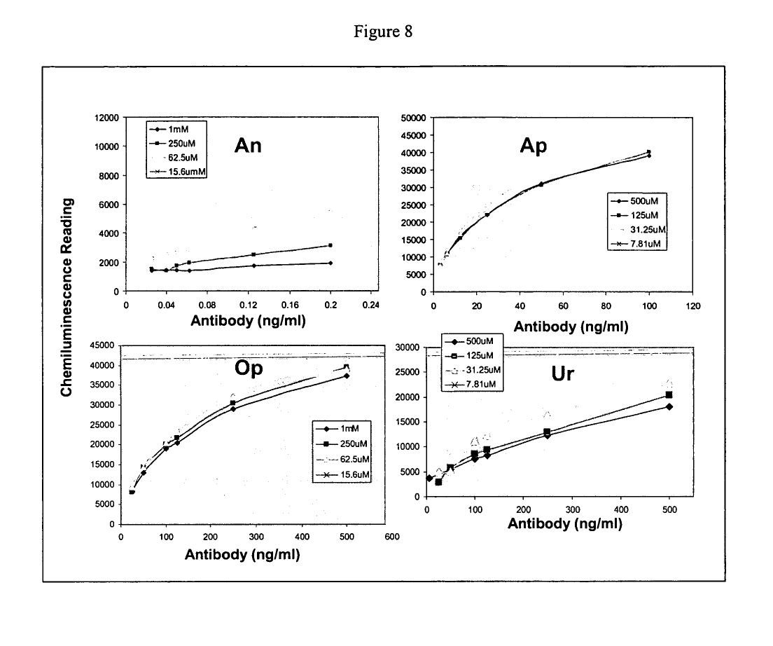

FIG. 8 shows the results of antibody titration curves for the above 4 antigens An, Ap, Op and Ur. The higher the competitor peptide concentration, the more effective/complete the competition, and thus the less ECL signals from the remaining bound primary capture agents.

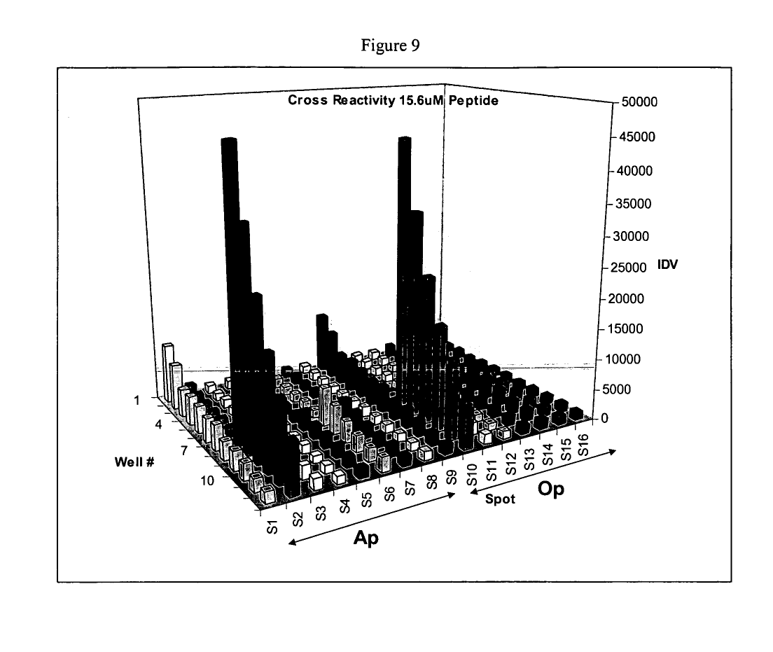

Antibody cross-reactivity can also be checked using a similar assay format. FIG. 9 demonstrates that the PET-specific antibodies used in FIGS. 7 and 8 are highly specific—they only reacts with different concentrations of the antigens to which they are raised against, but nothing else.

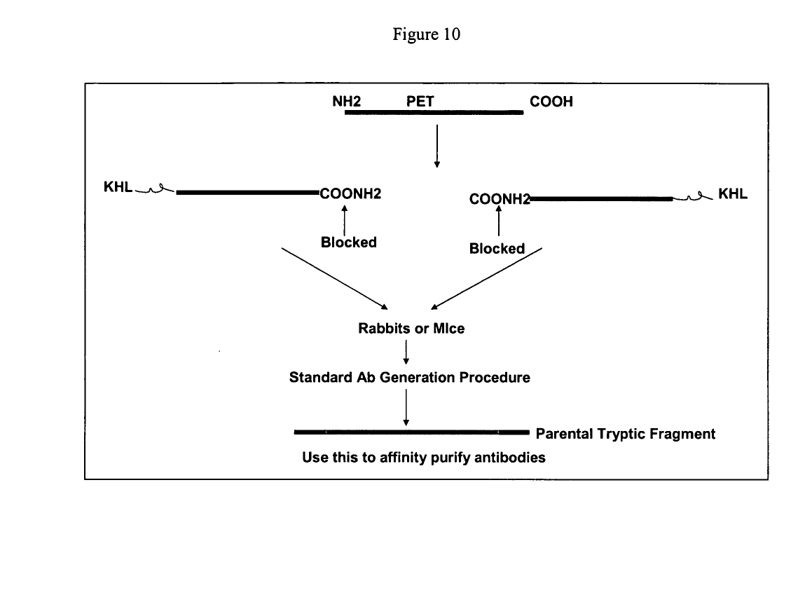

The PET antigens used for the generation of PET-specific antibodies are preferably blocked at either the N- or C-terminal end, most preferably at both ends (see FIG. 10) to generate neutral groups, since antibodies raised against peptides with non-neutralized ends may not be functional for the methods of the invention. The PET antigens can be most easily synthesized using standard molecular biology or chemical methods, for example, with a peptide synthesizer. The terminals can be blocked with NH2— or COO— groups as appropriate, or any other blocking agents to eliminate free ends. In a preferred embodiment, one end (either N- or C-terminus) of the PET will be conjugated with a carrier protein such as KHL or BSA to facilitate antibody generation. KHL represents Keyhole-limpet hemocyanin, an oxygen carrying copper protein found in the keyhole-limpet (Megathura crenulata), a primitive mollusk sea snail. KHL has a complex molecular arrangement and contains a diverse antigenic structure and elicits a strong nonspecific immune response in host animals. Therefore, when small peptides (which may not be very immunogenic) are used as immunogens, they are preferably conjugated to KHL or other carrier proteins (BSA) for enhanced immune responses in the host animal. The resulting antibodies can be affinity purified using a polypeptide corresponding to the PET-containing tryptic peptide of interest (see FIG. 10).

Blocking the ends of PET in antibody generation may be advantageous, since in many (if not most) cases, the selected PETs are contained within larger (tryptic) fragments. In these cases, the PET-specific antibodies are required to bind PETs in the middle of a peptide fragment. Therefore, blocking both the C- and N-terminus of the PETs best simulates the antibody binding of peptide fragments in a digested sample. Similarly, if the selected PET sequence happens to be at the N- or C-terminal end of a target fragment, then only the other end of the immunogen needs to be blocked, preferably by a carrier such as KHL or BSA.

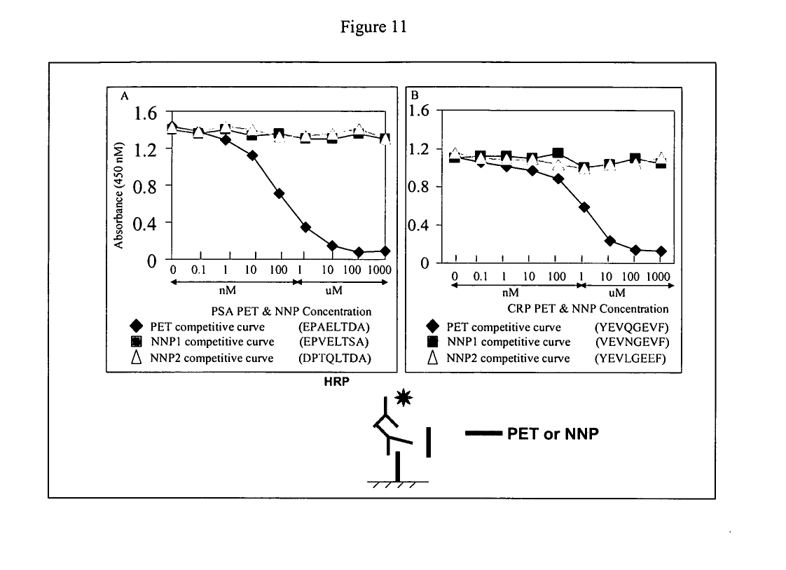

FIG. 11 below shows that PET-specific antibodies are highly specific and have high affinity for their respective PET-antigens.

B. Proteins and Peptides

Other methods for generating the capture agents of the present invention include phage-display technology described in, for example, Dower et al., WO 91/17271, McCafferty et al., WO 92/01047, Herzig et al., U.S. Pat. No. 5,877,218, Winter et al., U.S. Pat. No. 5,871,907, Winter et al., U.S. Pat. No. 5,858,657, Holliger et al., U.S. Pat. No. 5,837,242, Johnson et al., U.S. Pat. No. 5,733,743 and Hoogenboom et al., U.S. Pat. No. 5,565,332 (the contents of each of which are incorporated by reference). In these methods, libraries of phage are produced in which members display different antibodies, antibody binding sites, or peptides on their outer surfaces. Antibodies are usually displayed as Fv or Fab fragments. Phage displaying sequences with a desired specificity are selected by affinity enrichment to a specific analyte.

Methods such as yeast display and in vitro ribosome display may also be used to generate the capture agents of the present invention. The foregoing methods are described in, for example, Methods in Enzymology Vol 328-Part C: Protein-protein interactions & Genomics and Bradbury A. (2001) Nature Biotechnology 19:528-529, the contents of each of which are incorporated herein by reference.

In a related embodiment, proteins or polypeptides may also act as capture agents of the present invention. These peptide capture agents also specifically bind to a given analyte, and can be identified, for example, using phage display screening against an immobilized analyte, or using any other art-recognized methods. Once identified, the peptidic capture agents may be prepared by any of the well known methods for preparing peptidic sequences. For example, the peptidic capture agents may be produced in prokaryotic or eukaryotic host cells by expression of polynucleotides encoding the particular peptide sequence. Alternatively, such peptidic capture agents may be synthesized by chemical methods. Methods for expression of heterologous peptides in recombinant hosts, chemical synthesis of peptides, and in vitro translation are well known in the art and are described further in Maniatis et al., Molecular Cloning: A Laboratory Manual (1989), 2nd Ed., Cold Spring Harbor, N.Y.; Berger and Kimmel, Methods in Enzymology, Volume 152, Guide to Molecular Cloning Techniques (1987), Academic Press, Inc., San Diego, Calif.; Merrifield, J. (1969) J. Am. Chem. Soc. 91:501; Chaiken, I. M. (1981) CRC Crit. Rev. Biochem. 11:255; Kaiser et al. (1989) Science 243:187; Merrifield, B. (1986) Science 232:342; Kent, S. B. H. (1988) Ann. Rev. Biochem. 57:957; and Offord, R. E. (1980) Semisynthetic Proteins, Wiley Publishing, which are incorporated herein in their entirety by reference).

The peptidic capture agents may also be prepared by any suitable method for chemical peptide synthesis, including solution-phase and solid-phase chemical synthesis. Methods for chemically synthesizing peptides are well known in the art (see, e.g., Bodansky, M. Principles of Peptide Synthesis, Springer Verlag, Berlin (1993) and Grant, G. A (ed.). Synthetic Peptides: A User's Guide, W.H. Freeman and Company, New York (1992). Automated peptide synthesizers useful to make the peptidic capture agents are commercially available.

Protein capture agents may also be obtained for small molecules by engineering existing proteins using established computer algorithms. Looger et al. (Nature 423, 185-190, 2003) describes a computational design protocol that offers enormous generality for engineering protein structure and function. The structure-based computational method can drastically redesign protein ligand-binding specificities. This method was used to construct soluble receptors that bind small molecules such as trinitrotoluene, L-lactate or serotonin with high selectivity and affinity. The use of various ligands and proteins shows that a high degree of control over biomolecular recognition has been established computationally.

By using high-resolution three-dimensional structures, the algorithm identifies amino-acid sequences that are predicted to form a complementary surface between the protein and a target ligand replacing the wild-type ligand. The procedure combines target-ligand docking (10⁸ translations and rotations) with mutations of amino-acid residues in direct contact with the wild-type ligand (typically 12-18 residues, corresponding to 10⁴⁵ to 10⁶⁸ mutant structures representing 10¹⁵ to 10²³ sequences). The resulting combinatorial problem (10⁵³ to 10⁷⁶ choices) is solved with an algorithm based on the dead-end elimination (DEE) theorems. This procedure deterministically identifies the global minimum of a semi-empirical potential function describing the molecular interactions in the system, including a modified Lennard-Jones potential, an explicit, geometry-dependent hydrogen-bonding term and a continuum solvation term to represent the hydrophobic effect. Additionally, a new term demanding that potential hydrogen-bond donors and acceptors in the ligand must be satisfied was found to be critical; it captures the necessity of balancing the hydrogen bond inventory, which is a dominant effect in molecular recognition. Designs are selected for experimentation from a rank-ordered set of possibilities. The design process is relatively rapid, requiring about 3 days of computation to generate a set of designs in a particular protein for a given ligand on a 20-processor computer cluster. The detailed procedures of the method is described in the Supplemental Material section of Looger et al., Nature 423, 185-190, 2003 (incorporated herein by reference). This procedure was successfully used to engineer binding sites for trinitrotoluene (TNT), L-lactate or serotonin in place of the wild-type sugar or amino-acid ligands of five members of the Escherichia coli periplasmic binding protein (PBP) superfamily.

C. Scaffolded Peptides

An alternative approach to generating capture agents for use in the present invention makes use of antibodies are scaffolded peptides, e.g., peptides displayed on the surface of a protein. The idea is that restricting the degrees of freedom of a peptide by incorporating it into a surface-exposed protein loop could reduce the entropic cost of binding to a target protein, resulting in higher affinity. Thioredoxin, fibronectin, avian pancreatic polypeptide (aPP) and albumin, as examples, are small, stable proteins with surface loops that will tolerate a great deal of sequence variation. To identify scaffolded peptides that selectively bind a target analyte, libraries of chimeric proteins can be generated in which random peptides are used to replace the native loop sequence, and through a process of affinity maturation, those which selectively bind an analyte of interest are identified.

D. Simple Peptides and Peptidomimetic Compounds

Peptides are also attractive candidates for capture agents because they combine advantages of small molecules and proteins. Large, diverse libraries can be made either biologically or synthetically, and the “hits” obtained in binding screens against a particular analyte can be made synthetically in large quantities.

Peptide-like oligomers (Soth et al. (1997) Curr. Opin. Chem. Biol. 1:120-129) such as peptoids (Figliozzi et al., (1996) Methods Enzymol. 267:437-447) can also be used as capture reagents, and can have certain advantages over peptides. They are impervious to proteases and their synthesis can be simpler and cheaper than that of peptides, particularly if one considers the use of functionality that is not found in the 20 common amino acids.

E. Nucleic Acids

In another embodiment, aptamers binding specifically to an analyte may also be used as capture agents. As used herein, the term “aptamer,” e.g., RNA aptamer or DNA aptamer, includes single-stranded oligonucleotides that bind specifically to a target molecule. Aptamers are selected, for example, by employing an in vitro evolution protocol called systematic evolution of ligands by exponential enrichment. Aptamers bind tightly and specifically to target molecules; most aptamers to proteins bind with a K_{d} (equilibrium dissociation constant) in the range of 1 pM to 1 nM. Aptamers and methods of preparing them are described in, for example, E. N. Brody et al. (1999) Mol. Diagn. 4:381-388, the contents of which are incorporated herein by reference.

In one embodiment, the subject aptamers can be generated using SELEX, a method for generating very high affinity receptors that are composed of nucleic acids instead of proteins. See, for example,. Brody et al. (1999) Mol. Diagn. 4:381-388. SELEX offers a completely in vitro combinatorial chemistry alternative to traditional protein-based antibody technology. Similar to phage display, SELEX is advantageous in terms of obviating animal hosts, reducing production time and labor, and simplifying purification involved in generating specific binding agents to a particular target analyte.

To further illustrate, SELEX can be performed by synthesizing a random oligonucleotide library, e.g., of greater than 20 bases in length, which is flanked by known primer sequences. Synthesis of the random region can be achieved by mixing all four nucleotides at each position in the sequence. Thus, the diversity of the random sequence is maximally 4ⁿ, where n is the length of the sequence, minus the frequency of palindromes and symmetric sequences. The greater degree of diversity conferred by SELEX affords greater opportunity to select for oligonuclotides that form 3-dimensional binding sites. Selection of high affinity oligonucleotides is achieved by exposing a random SELEX library to an immobilized target analyte. Sequences, which bind readily without washing away, are retained and amplified by the PCR, for subsequent rounds of SELEX consisting of alternating affinity selection and PCR amplification of bound nucleic acid sequences. Four to five rounds of SELEX are typically sufficient to produce a high affinity set of aptamers.

Therefore, hundreds to thousands of aptamers can be made in an economically feasible fashion. Blood and urine can be analyzed on aptamer chips that capture and quantitate proteins. SELEX has also been adapted to the use of 5-bromo (5-Br) and 5-iodo (5-I) deoxyuridine residues. These halogenated bases can be specifically cross-linked to proteins. Selection pressure during in vitro evolution can be applied for both binding specificity and specific photo-cross-linkability. These are sufficiently independent parameters to allow one reagent, a photo-cross-linkable aptamer, to substitute for two reagents, the capture antibody and the detection antibody, in a typical sandwich array. After a cycle of binding, washing, cross-linking, and detergent washing, proteins will be specifically and covalently linked to their cognate aptamers. Because no other proteins are present on the chips, protein-specific stain will now show a meaningful array of pixels on the chip. Combined with learning algorithms and retrospective studies, this technique should lead to a robust yet simple diagnostic chip.

In yet another related embodiment, a capture agent may be an allosteric ribozyme. The term “allosteric ribozymes,” as used herein, includes single-stranded oligonucleotides that perform catalysis when triggered with a variety of effectors, e.g., nucleotides, second messengers, enzyme cofactors, pharmaceutical agents, proteins, and oligonucleotides. Allosteric ribozymes and methods for preparing them are described in, for example, S. Seetharaman et al. (2001) Nature Biotechnol. 19: 336-341, the contents of which are incorporated herein by reference. According to Seetharaman et al., a prototype biosensor array has been assembled from engineered RNA molecular switches that undergo ribozyme-mediated self-cleavage when triggered by specific effectors. Each type of switch is prepared with a 5′-thiotriphosphate moiety that permits immobilization on gold to form individually addressable pixels. The ribozymes comprising each pixel become active only when presented with their corresponding effector, such that each type of switch serves as a specific analyte sensor. An addressed array created with seven different RNA switches was used to report the status of targets in complex mixtures containing metal ion, enzyme cofactor, metabolite, and drug analytes. The RNA switch array also was used to determine the phenotypes of Escherichia coli strains for adenylate cyclase function by detecting naturally produced 3′,5′-cyclic adenosine monophosphate (cAMP) in bacterial culture media.

F. Plastibodies

In certain embodiments the subject capture agent is a plastibody. The term “plastibody” refers to polymers imprinted with selected template molecules. See, for example, Bruggemann (2002) Adv Biochem Eng Biotechnol 76:127-63; and Haupt et al. (1998) Trends Biotech. 16:468-475. The plastibody principle is based on molecular imprinting, namely, a recognition site that can be generated by stereoregular display of pendant functional groups that are grafted to the sidechains of a polymeric chain to thereby mimic the binding site of, for example, an antibody.

G. Chimeric Binding Agents Derived From Two Low-Affinity Ligands

Still another strategy for generating suitable capture agents is to link two or more modest-affinity ligands and generate high affinity capture agent. Given the appropriate linker, such chimeric compounds can exhibit affinities that approach the product of the affinities for the two individual ligands for the analyte (e.g. PET peptide). To illustrate, a collection of compounds is screened at high concentrations for weak interacters of a target analyte. The compounds that do not compete with one another are then identified and a library of chimeric compounds is made with linkers of different length. This library is then screened for binding to the analyte at much lower concentrations to identify high affinity binders. Such a technique may also be applied to peptides or any other type of modest-affinity analyte-binding compound.

H. Labels for Capture Agents

The capture agents of the present invention may be modified to enable detection using techniques known to one of ordinary skill in the art, such as fluorescent, radioactive, chromatic, optical, and other physical or chemical labels, as described herein below.

I. Miscellaneous

In addition, for any given analyte, multiple capture agents belonging to each of the above described categories of capture agents may be available. These multiple capture agents may have different properties, such as affinity/avidity/specificity for the analyte. Different affinities are useful in covering the wide dynamic ranges of expression which some binders can exhibit. Depending on specific use, in any given array of capture agents, different types/amounts of capture agents may be present on a single chip/array to achieve optimal overall performance.

In a preferred embodiment, capture agents are raised against PETs that are located on the surface of the protein of interest, e.g., hydrophilic regions. PETs that are located on the surface of the protein of interest may be identified using any of the well known software available in the art. For example, the Naccess program may be used.

Naccess is a program that calculates the accessible area of a molecule from a PDB (Protein Data Bank) format file. It can calculate the atomic and residue accessibilities for both proteins and nucleic acids. Naccess calculates the atomic accessible area when a probe is rolled around the Van der Waal's surface of a macromolecule. Such three-dimensional co-ordinate sets are available from the PDB at the Brookhaven National laboratory. The program uses the Lee & Richards (1971) J. Mol. Biol., 55, 379400 method, whereby a probe of given radius is rolled around the surface of the molecule, and the path traced out by its center is the accessible surface.

The solvent accessibility method described in Boger, J., Emini, E. A. & Schmidt, A., Surface probability profile—An heuristic approach to the selection of synthetic peptide antigens, Reports on the Sixth International Congress in Immunology (Toronto) 1986 p. 250 also may be used to identify PETs that are located on the surface of the protein of interest. The package MOLMOL (Koradi, R. et al. (1996) J. Mol. Graph. 14:51-55) and Eisenhaber's ASC method (Eisenhaber and Argos (1993) J. Comput. Chem. 14:1272-1280; Eisenhaber et al. (1995) J. Comput. Chem. 16:273-284) may also be used.

In another embodiment, capture agents are raised that are designed to bind with peptides generated by digestion of intact proteins rather than with accessible peptidic surface regions on the proteins. In this embodiment, it is preferred to employ a fragmentation protocol which reproducibly generates all of the PETs in the sample under study.

IV. Array Construction

In certain embodiments, to construct arrays, e.g., high-density arrays, the target analytes (e.g. PET peptide fragments) need to be immobilized onto a solid support (e.g., a planar support or a bead). A variety of methods are known in the art for attaching biological molecules to solid supports. See, generally, Affinity Techniques, Enzyme Purification: Part B, Meth. Enz. 34 (ed. W. B. Jakoby and M. Wilchek, Acad. Press, N.Y. 1974) and Immobilized Biochemicals and Affinity Chromatography, Adv. Exp. Med. Biol. 42 (ed. R. Dunlap, Plenum Press, N.Y. 1974). The following are a few considerations when constructing arrays.

A. Formats and Surfaces Consideration

Arrays have been designed as a miniaturization of familiar immunoassay methods such as ELISA and dot blotting, often utilizing fluorescent readout, and facilitated by robotics and high throughput detection systems to enable multiple assays to be carried out in parallel. Common physical supports include glass slides, silicon, microwells, nitrocellulose or PVDF membranes, and magnetic and other microbeads. While microdrops of protein delivered onto planar surfaces are widely used, related alternative architectures include CD centrifugation devices based on developments in microfluidics [Gyros] and specialized chip designs, such as engineered microchannels in a plate [The Living Chip™, Biotrove] and tiny 3 D posts on a silicon surface [Zyomyx]. Particles in suspension can also be used as the basis of arrays, providing they are coded for identification; systems include color coding for microbeads [Luminex, Bio-Rad] and semiconductor nanocrystals [QDots™, Quantum Dots], and barcoding for beads [UltraPlex™, Smartbeads] and multimetal microrods [Nanobarcodes™ particles, Surromed]. Beads can also be assembled into planar arrays on semiconductor chips [LEAPS technology, BioArray Solutions].

B. Immobilization Considerations

For small molecule immobilization, Winssinger et al. (“From split-pool libraries to spatially addressable microarrays and its application to functional proteomic profiling,” Angewandte Chemie International Edition in English, 40:3152-55, 2001, incorporate herein by reference) recently reported a simple, general and robust new technique that utilizes the ability of peptide nucleic acid (PNA) to bind strongly to microarrays of encoding tags in the form of DNA to pull out high affinity ligands for different proteins in mixtures. In that report, small molecules are synthesized simultaneously with an encoding PNA string and incubated with target proteins in solution. The complexes are isolated by simple dialysis and structure of active ligands decoded by binding to complementary DNA codes on the microchip. Detection of protein binding by differential fluorescence labeled target proteins allows the distinction between binding activities for several targets. The same technology can be readily modified to immobilize large amounts of different small molecules in array format. Briefly, a tag PNA of a specific sequence may be covalently attached to each small molecule of interest. The PNA tag will then specifically tether the linked small molecule to an addressable location on a microarray, by hybridizing specifically with matching polynucleotide sequences immobilized on the array.

An added advantage of using the PNA tag is that all small molecules on an array are similarly oriented, thus providing more consistent and more standardized binding between the small molecules and their capture agents.

Similarly, a DNA, rather than a PNA tag may be used for the same purpose.

Alternatively, small molecules may be printed directly onto solid support to manufacture microarrays. In order to allow attachment by an adapter or directly by a small molecule, the surface of the substrate may require preparation to create suitable reactive groups. Such reactive groups could include simple chemical moieties such as amino, hydroxyl, carboxyl, carboxylate, aldehyde, ester, amide, amine, nitrile, sulfonyl, phosphoryl, or similarly chemically reactive groups. Alternatively, reactive groups may comprise more complex moieties that include, but are not limited to, sulfo-N-hydroxysuccinimide, nitrilotriacetic acid, activated hydroxyl, haloacetyl (e.g., bromoacetyl, iodoacetyl), activated carboxyl, hydrazide, epoxy, aziridine, sulfonylchloride, trifluoromethyldiaziridine, pyridyldisulfide, N-acyl-imidazole, imidazolecarbamate, succinimidylcarbonate, arylazide, anhydride, diazoacetate, benzophenone, isothiocyanate, isocyanate, imidoester, fluorobenzene, biotin and avidin. Techniques of placing such reactive groups on a substrate by mechanical, physical, electrical or chemical means are well known in the art, such as described by U.S. Pat. No. 4,681,870, incorporated herein by reference.

Once the initial preparation of reactive groups on the substrate is completed (if necessary), adapter molecules optionally may be added to the surface of the substrate to make it suitable for further attachment chemistry. Such adapters covalently join the reactive groups already on the substrate and the small molecules to be immobilized, having a backbone of chemical bonds forming a continuous connection between the reactive groups on the substrate and the small molecules, and having a plurality of freely rotating bonds along that backbone. Substrate adapters may be selected from any suitable class of compounds and may comprise polymers or copolymers of organic acids, aldehydes, alcohols, thiols, amines and the like. For example, polymers or copolymers of hydroxy-, amino-, or di-carboxylic acids, such as glycolic acid, lactic acid, sebacic acid, or sarcosine may be employed. Alternatively, polymers or copolymers of saturated or unsaturated hydrocarbons such as ethylene glycol, propylene glycol, saccharides, and the like may be employed. Preferably, the substrate adapter should be of an appropriate length to allow the small molecule, which is to be attached, to interact freely with molecules (such as capture agents) in a sample solution and to form effective binding. The substrate adapters may be either branched or unbranched, but this and other structural attributes of the adapter should not interfere stereochemically with relevant functions of the immobilized small molecules, such as a binding to the capture agent. Protection groups, known to those skilled in the art, may be used to prevent the adapter's end groups from undesired or premature reactions. For instance, U.S. Pat. No. 5,412,087, incorporated herein by reference, describes the use of photo-removable protection groups on a adapter's thiol group.

Methods of coupling the analytes to the reactive end groups on the surface of the substrate or on the adapter include reactions that form linkage such as thioether bonds, disulfide bonds, amide bonds, carbamate bonds, urea linkages, ester bonds, carbonate bonds, ether bonds, hydrazone linkages, Schiff-base linkages, and noncovalent linkages mediated by, for example, ionic or hydrophobic interactions. The form of reaction will depend, of course, upon the available reactive groups on both the substrate/adapter and the small molecule to be immobilized.

To illustrate, Stuart Schreiber's laboratory has pursued several different types of chemistry for covalent attachment of small molecules to glass microscope slides with success. Herein below describes several most commonly used surfaces that may be used to immobilize thiols, primary alcohols, phenols, and carboxylic acids in generating small molecule microarrays.

One of the favored attachment method for small molecules involves primary and secondary alcohols (chlorinated glass) or phenols (diazobenzylidene-functionalized glass). This chemistry is compatible with diversity-oriented synthesis (such as split pool synthesis) that uses high-capacity 500-600 μM polystyrene beads equipped with a silicon linker for temporary attachment and eventual fluoride-mediated release of synthetic, alcohol-containing compounds. This strategy has been used to prepare and print more than 40,000 small molecules from ten different DOS-libraries including 1,3-dioxanes,6,7 dihydropyrancarboxamides, 8,9 and biaryl-containing medium rings (Spring et al., J. Am. Chem. Soc. 124: 1354-1363, 2002).

Fabrication of Custom Slide Chambers: In an effort to minimize reagent volume during the chemical treatment of glass microscope slides, custom slide-sized reaction chambers can be designed and fabricated. In one embodiment, the chambers enable the uniform application of 1.35 mL to one face of a 2.5 cm×7.5 cm glass slide. Each chamber can hold two slides. A master template mold designed to hold, e.g., two arrays (or any other desired number of arrays) is cut from a block of Delhran plastic. The chambers are prepared by casting degassed polydimethylsiloxane prepolymer around the master template in a polystyrene OmniTray. After curing at 65° C. for 4 hours, the polymer is peeled away from the master template to give the finished product. Microscope slides are placed in the chambers with the face to be modified down. Reagents are introduced under the slides and to the reactive face.

Cleaning Glass Slides: To make amino-functionalized slides or activating slides with thionyl chloride, plain glass slides (cat. # 48300-036) can be purchased from VWR Scientific Products, USA (other any other suitable vender) and cleaned in piranha solution (70:30 v/v mixture of concentrated H₂SO₄ to 30% H₂O₂) for at least 12 hours at room temperature. Once the slides are removed from the piranha bath, they are washed for at least 12 hours in ddH₂O. The slides are stored in ddH₂O until further use.

Preparation of Amino-Functionalized Glass Slides: Cleaned slides are removed from water and dried by centrifugation. A 200 mL solution containing 3:5:92 3-aminopropyltriethoxysilane: ddH₂O:ethanol is prepared and stirred for 10 minutes to allow for hydrolysis and formation of silanol. The silanol solution is poured into a 250 mL glass slide tank containing the cleaned glass slides and a stir bar. The slides are incubated in the solution with stirring for 1 hour at room temperature. The slides were removed from the silanol solution, washed for 30 seconds in 100% ethanol, and dried by centrifugation to remove excess silanol from the surface.

The adsorbed silane layer is cured at 115° C. for 1 hour. After cooling to room temperature, the slides are washed in 95% (v/v) ethanol for 30 minutes. The washing is repeated four times. Amino slides are stored under vacuum at room temperature until further use. One slide from each batch is used to verify the presence of amino groups on the glass surface. The slide is washed briefly in 5 mL 50 mM sodium bicarbonate, pH 8.5. The slide is then dipped in 5 mL of 50 mM sodium bicarbonate, pH 8.5 containing 2% (v/v) DMF and 0.1 mM sulfo-succinimidyl-4-O-(4,4′-dimethoxytrityl)-butyrate (s-SDTB). The slide is incubated in the s-SDTB solution with shaking for 30 minutes at room temperature. The slide is then washed three times in 20 mL of ddH₂O and subsequently treated with 5 mL of 30% (v/v) perchloric acid. An orange-colored solution indicated that the slide had been successfully derivatized with amines. No color change is observed for untreated glass slides. Quantitation of the 4,4′-dimethoxytrityl cation (e498 nm=70,000 M-1 cm-1) released by acid treatment indicated an approximate density of 2-4 amino groups per nm².

Preparation of Michael Acceptor-Functionalized Glass Slides For Capture of Thiol-Containing Small Molecules: Amino-functionalized slides (CMT-GAPS™ coated or prepared as described above) are transferred to the custom polydimethylsiloxane (PDMS) slide chambers. Several different types of Michael acceptor slides are prepared by treating one face of each slide with a 20 mM solution of one the reagents. Solutions of NHS-esters are prepared by dissolving in DMF and then diluting 10-fold with 50 mM sodium bicarbonate buffer, pH 8.5. Alternatively, solutions of NHS-esters are prepared by dissolving in DMF containing 5 eq. DIPEA. Succinimidyl ester 8 is prepared according to the procedure of Nielsen et al. in comparable yield. The slides are incubated in these solutions for 3 hours at room temperature. Slides are then washed four times in ddH₂O for 30 minutes each, dried by centrifugation, and stored at room temperature under vacuum until further use.

Preparation of Silyl Chloride Glass Slides For Capture of Primary Alcohol-Containing Small Molecules: Standard glass microscope slides are cleaned as described above. To convert to the silyl chloride, the slides are first removed from water and dried by centrifugation. The dried slides are then immersed in a solution of dry THF containing 1% (v/v) thionyl chloride and 0.1% DMF in a glass slide tank (oven-dried overnight). The slides are incubated in this solution for 4 hours at room temperature. The slides are then removed from the chlorination solution, washed briefly in THF, and immediately placed on the microarrayer platform for printing.

Preparation of Diazobenzylidene Glass Slides For Capture of Phenols and Carboxylic Acids: Diazobenzylidene slides were prepared as follows. CMT-GAPS™ coated slides (Corning®) or homemade amino slides are immersed in a solution of 1 (10 mM), PyBOP (10 mM), and DIPEA (10 mM) in anhydrous DMF for 2-16 hours (2 hours is sufficient, 16 hours is typical). The slides are then washed extensively in DMF and then in methanol. To convert the tosylhydrazone-derived slides to diazobenzylidenederived slides, the slides are immersed in a solution of 100 mM sodium methoxide in ethylene glycol, and heated at 90° C. for 2 hours. The slides are washed extensively with methanol. The slides can be stored at this stage for at least 3 weeks in the dark at room temperature with no noticeable deterioration in performance, but are usually stored at −20° C.

Synthesis of 1,4-carboxybenzaldehyde (50.5 g, 336 mmol) and toluenesulfonylhydrazide (62.5 g, 336 mmol) are heated in methanol (1.5 L) at 70° C. The resulting solution is stirred at 23° C. for 16 hours, brought to 60° C. and, after addition of 750 mL water, is slowly cooled to 23° C. The white precipitate (69.7 g) is collected by filtration. Water (2 L) is added to the filtrate, and the resulting precipitate (31.9 g) is collected by filtration to afford 1 (101.6 g, 95%): ¹H NMR (400 MHz, CD₃OD) d 7.97 (d, J=8.4 Hz, 2H), 7.85 (s, 1H), 7.82 (d, J=8.0 Hz, 2H), 7.64 (d, J=8.4 Hz, 2H), 7.35 (d, J=8.0 Hz, 2H), 2.37 (s, 3H); ¹³C NMR (100 MHz, CD₃OD) d 169.2, 147.1, 145.5, 139.5, 137.3, 133.0, 131.0, 130.7, 128.7, 127.9, 21.5; FT-IR (thin film) 3216 (br), 1699, 1686, 1673, 1664, 1654, 1555, 1509, 1412, 1366, 1346, 1320, 1289, 1228, 1157, 1121, 1049, 1013, 942, 840, 768, 697 cm⁻¹; LCMS (TOF ES) calcd for C₁₅H₁₅N₂O₄, 319 m/z (M+H)⁺; observed 319.

Preparation of Tetramethylrhodamine Marker (4a) on Polystyrene Beads with a 6-aminocaproic acid Linker (7a): Either Polystyrene A Trt-Cys(Mmt) Fmoc or Polystyrene A Trt-Ala Fmoc resin (400 mg, 0.4 meq/g, 0.16 mmol) is placed in a 10 mL column and allowed to swell in 6 mL DMF for 2 minutes. The column is drained and the Fmoc group is removed by two 15 minute treatments with 6 mL of 20% (v/v) piperidine in DMF. The resin is washed as described for the general procedures, dried under vacuum, and swollen with 6 mL of anhydrous DMF for 2 minutes. The column is drained and the resin is swollen with 6 mL of distilled CH₂Cl₂ for another 2 minutes. The column is drained and a solution of Fmoc-w-Aca-OH (238 mg, 0.8 mmol, 5 eq.) and PyBOP® (416 mg, 0.8 mmol, 5 eq.) in 5.2 mL anhydrous DMF is added to the resin. The column is rocked gently to mix the contents and then DIPEA (279 μL, 1.60 mmol, 10 eq.) is added. After rocking gently for 12 hours, the resin is washed and provided a negative Kaiser ninhydrin test result. The Fmoc group is then removed as described above, washed, and dried under vacuum. At this point, the resin provides a positive Kaiser test result.

Resin 7a (80 mg, 0.032 mmol, 1 eq.) is placed into a 2 mL column and swollen with 1.5 mL anhydrous DMF for 2 minutes. The column is drained, the resin is swollen with 1.5 mL distilled CH₂Cl₂ for another 2 minutes, and drained again. A solution of 5(6)-TAMRA succinimidyl ester (50 mg, 0.094 mmol, 3.0 eq.) and DIPEA (40 μL, 0.23 mmol, 7.2 eq.) in 1.0 mL anhydrous DMF is added to the column. The resin is agitated by gentle rocking for 12 hours, drained and washed. The resin gives a negative Kaiser test result. An aliquot of beads (10 mg) is exposed to 100 μL of a solution containing 2:1:17 TFA:TIS:CHCl₃ for 2 hours to cleave compound from the resin and to deprotect the Mmt-protected thiol of 4a. The cleavage solution is removed in vacuo and the crude products are dissolved in 10 μL of acetonitrile for analysis by LCMS.

(9-{2-Carboxyl-5-[5-(1R-carboxy-2-mercapto-ethylcarbamoyl)-pentacarbamoyl]-phenyl}-6-dimethylamino-xanthen-3-ylidene)-dimethyl-ammonium and (9-{2-Carboxyl-5-[6-(1R-carboxy-2-mercapto-ethylcarbamoyl)-pentacarbamoyl]-phenyl}-6-dimethylamino-xanthen-3-ylidene)-dimethyl-ammonium (5,6-TAMRA-w-Aca-Cys, 4a). LCMS (TOF MS ES⁺): tR=8.126 min., m/z (rel int) 647 ([M+H]⁺, 100). HRMS (NBA/NaI) m/z calcd for C₃₄H₃₈N₄O₇SNa 669.7429; found 669.7432.

Small molecules are printed onto activated slides using the OmniGrid™ 2000 Microarrayer (GeneMachines, San Carlos, Calif.). The microarrayer is loaded with 48 ArrayIt™ stealth microspotting pins (catalog # SMP4, TeleChem International, Inc., Sunnyvale, Calif.). The pins typically pick up 250 nL of the DMF stock solution from a 384-well microtiter plate. To ensure uniform spot diameters, ca. 20 spots were printed on a blot slide or a series of 20 unactivated blot slides at the front of the platter. The arrayer is instructed to deliver 1 nL drops placed 350-375 μM apart on the slides. The pins are washed with acetonitrile (or acetone) in a stirring bath for 8 seconds and dried under a stream of air for 8 seconds. The cycle is repeated before dipping into the next well for a 6 second sample loading. Tetramethylrhodamine marker 4a is printed on thionyl chloride and maleimide slides as a marker. The marker is placed in the upper right hand corner of each 12×12 feature subarray (48 such subarrays make up the 6,912-feature total array).

Following printing, the maleimide slides are left on the printing platter at room temperature for 12 hours and then immersed in a 1% (v/v) solution of 2-mercaptoethanol in DMF to quench any remaining maleimide groups. Silyl chloride slides and diazobenzylidene slides are also allowed to sit undisturbed on the platter for 12 hours after printing. Diazobenzylidene slides are then immersed in a 1 M aq. glycolic acid solution for 30 minutes to quench any remaining diazobenzylidene moieties. A quench step is not performed for thionyl chloride slides. All slides are then washed for at least 1 hour each in DMF, THF, and iso-propanol or methanol. Slides are dried by centrifugation, and either used immediately or stored in a foil-covered box, flushed with argon at −20° C.

The variables in immobilization of proteins such as PET-containing peptide fragments include both the coupling reagent and the nature of the surface being coupled to. Ideally, the immobilization method used should be reproducible, applicable to proteins of different properties (size, hydrophilic, hydrophobic), amenable to high throughput and automation, and compatible with retention of fully functional protein activity. Orientation of the surface-bound protein is recognized as an important factor in presenting it to ligand or substrate in an active state; for peptide arrays the most efficient binding results are obtained with orientated peptide fragments, which generally requires site-specific labeling of the protein.

The properties of a good protein array support surface are that it should be chemically stable before and after the coupling procedures, allow good spot morphology, display minimal nonspecific binding, not contribute a background in detection systems, and be compatible with different detection systems.

Both covalent and noncovalent methods of protein immobilization are used and have various pros and cons. Passive adsorption to surfaces is methodologically simple, but allows little quantitative or orientational control; it may or may not alter the functional properties of the protein, and reproducibility and efficiency are variable. Covalent coupling methods provide a stable linkage, can be applied to a range of proteins and have good reproducibility; however, orientation may be variable, chemical dramatization may alter the function of the protein and requires a stable interactive surface. Biological capture methods utilizing a tag on the protein provide a stable linkage and bind the protein specifically and in reproducible orientation, but the biological reagent must first be immobilized adequately and the array may require special handling and have variable stability.

Several immobilization chemistries and tags have been described for fabrication of protein arrays. Substrates for covalent attachment include glass slides coated with amino- or aldehyde-containing silane reagents [Telechem]. In the Versalinx™ system [Prolinx], reversible covalent coupling is achieved by interaction between the protein derivatized with phenyldiboronic acid, and salicylhydroxamic acid immobilized on the support surface. This also has low background binding and low intrinsic fluorescence and allows the immobilized proteins to retain function. Noncovalent binding of unmodified protein occurs within porous structures such as HydroGel™ [PerkinElmer], based on a 3-dimensional polyacrylamide gel; this substrate is reported to give a particularly low background on glass microarrays, with a high capacity and retention of protein function. Widely used biological capture methods are through biotin/streptavidin or hexahistidine/Ni interactions, having modified the protein appropriately. Biotin may be conjugated to a poly-lysine backbone immobilized on a surface such as titanium dioxide [Zyomyx] or tantalum pentoxide [Zeptosens].

Arenkov et al., for example, have described a way to immobilize proteins while preserving their function by using microfabricated polyacrylamide gel pads to proteins, and then accelerating diffusion through the matrix by microelectrophoresis (Arenkov et al. (2000), Anal Biochem 278(2):123-31). The patent literature also describes a number of different methods for attaching biological molecules to solid supports. For example, U.S. Pat. No. 4,282,287 describes a method for modifying a polymer surface through the successive application of multiple layers of biotin, avidin, and extenders. U.S. Pat. No. 4,562,157 describes a technique for attaching biochemical ligands to surfaces by attachment to a photochemically reactive arylazide. U.S. Pat. No. 4,681,870 describes a method for introducing free amino or carboxyl groups onto a silica matrix, in which the groups may subsequently be covalently linked to a protein in the presence of a carbodiimide. In addition, U.S. Pat. No. 4,762,881 describes a method for attaching a polypeptide chain to a solid substrate by incorporating a light-sensitive unnatural amino acid group into the polypeptide chain and exposing the product to low-energy UV light.

The surface of the support is chosen to possess, or is chemically derivatized to possess, at least one reactive chemical group that can be used for further attachment chemistry. There may be optional flexible adapter molecules interposed between the support and the capture agents. In one embodiment, the capture agents are physically adsorbed onto the support.

In certain embodiments of the invention, a PET-containing peptide is immobilized on a support in ways that separate the PET region used to bind capture agents and the region where it is linked to the support. In a preferred embodiment, the PET-containing peptide is engineered to form a covalent bond between one of its termini to an adapter molecule on the support. Such a covalent bond may be formed through a Schiff-base linkage, a linkage generated by a Michael addition, or a thioether linkage.

In order to allow attachment by an adapter or directly by a PET-containing peptide, the surface of the substrate may require preparation to create suitable reactive groups. Generally see above, including those described by U.S. Pat. No. 4,681,870, incorporated herein by reference.

C. Array Fabrication Consideration

Preferably, the immobilized small molecules or PET sequences are arranged in an array on a solid support, such as a silicon-based chip or glass slide. One or more small molecules or PET sequences designed to detect the presence and the concentration of a given target (one previously recognized as existing) is immobilized at each of a plurality of cells/regions/addressable locations in the array. Thus, a signal at a particular cell/region/location indicates the presence of a known target in the sample, and the identity of the protein is revealed by the position of the cell. Alternatively, small molecules or PET sequences are immobilized on beads, which optionally are labeled to identify their intended target analyte, or are distributed in an array such as a microwell plate.

In one embodiment, the microarray is high density, with a density over about 100, preferably over about 1000, 1500, 2000, 3000, 4000, 5000 and further preferably over about 9000, 10000, 11000, 12000 or 13000 spots per cm², formed by attaching small molecules or PET sequences onto a support surface which has been functionalized to create a high density of reactive groups or which has been functionalized by the addition of a high density of adapters bearing reactive groups. In another embodiment, the microarray comprises a relatively small number of small molecules or PET sequences, e.g., 10 to 50, selected to detect in a sample various combinations of specific proteins which generate patterns probative of disease diagnosis, cell type determination, pathogen identification, etc.

Although the characteristics of the substrate or support may vary depending upon the intended use, the shape, material and surface modification of the substrates must be considered. Although it is preferred that the substrate have at least one surface which is substantially planar or flat, it may also include indentations, protuberances, steps, ridges, terraces and the like and may have any geometric form (e.g., cylindrical, conical, spherical, concave surface, convex surface, string, or a combination of any of these). Suitable substrate materials include, but are not limited to, glasses, ceramics, plastics, metals, alloys, carbon, papers, agarose, silica, quartz, cellulose, polyacrylamide, polyamide, and gelatin, as well as other polymer supports, other solid-material supports, or flexible membrane supports. Polymers that may be used as substrates include, but are not limited to: polystyrene; poly(tetra)fluoroethylene (PTFE); polyvinylidenedifluoride; polycarbonate; polymethylmethacrylate; polyvinylethylene; polyethyleneimine; polyoxymethylene (POM); polyvinylphenol; polylactides; polymethacrylimide (PMI); polyalkenesulfone (PAS); polypropylene; polyethylene; polyhydroxyethylmethacrylate (HEMA); polydimethylsiloxane; polyacrylamide; polyimide; and various block co-polymers. The substrate can also comprise a combination of materials, whether water-permeable or not, in multi-layer configurations. A preferred embodiment of the substrate is a plain 2.5 cm×7.5 cm glass slide with surface Si—OH functionalities.

Array fabrication methods include robotic contact printing, ink-jetting, piezoelectric spotting and photolithography. A number of commercial arrayers are available [e.g. Packard Biosience] as well as manual equipment [V & P Scientific]. Bacterial colonies can be robotically gridded onto PVDF membranes for induction of protein expression in situ.

At the limit of spot size and density are nanoarrays, with spots on the nanometer spatial scale, enabling thousands of reactions to be performed on a single chip less than 1 mm square. BioForce Laboratories have developed nanoarrays with 1521 protein spots in 85 sq microns, equivalent to 25 million spots per sq cm, at the limit for optical detection; their readout methods are fluorescence and atomic force microscopy (AFM).

A microfluidics system for automated sample incubation with arrays on glass slides and washing has been codeveloped by NextGen and PerkinElmer Lifesciences.

For example, the subject microarrays may be produced by a number of means, including “spotting” wherein small amounts of the reactants are dispensed to particular positions on the surface of the substrate. Methods for spotting include, but are not limited to, microfluidics printing, microstamping (see, e.g., U.S. Pat. No. 5,515,131, U.S. Pat. No. 5,731,152, Martin, B. D. et al. (1998), Langmuir 14: 3971-3975 and Haab, B B et al. (2001) Genome Biol 2 and MacBeath, G. et al. (2000) Science 289: 1760-1763), microcontact printing (see, e.g., PCT Publication WO 96/29629), inkjet head printing (Roda, A. et al. (2000) BioTechniques 28: 492-496, and Silzel, J. W. et al. (1998) Clin Chem 44: 2036-2043), microfluidic direct application (Rowe, C. A. et al. (1999) Anal Chem 71: 433-439 and Bernard, A. et al. (2001), Anal Chem 73: 8-12) and electrospray deposition (Morozov, V. N. et al. (1999) Anal Chem 71: 1415-1420 and Moerman R. et al. (2001) Anal Chem 73: 2183-2189). Generally, the dispensing device includes calibrating means for controlling the amount of sample deposition, and may also include a structure for moving and positioning the sample in relation to the support surface. The volume of fluid to be dispensed per target molecule in an array varies with the intended use of the array, and available equipment. Preferably, a volume formed by one dispensation is less than 100 nL, more preferably less than 10 nL, and most preferably about 1 nL. The size of the resultant spots will vary as well, and in preferred embodiments these spots are less than 20,000 μm in diameter, more preferably less than 2,000 μm in diameter, and most preferably about 150-200 μm in diameter (to yield about 1600 spots per square centimeter). Solutions of blocking agents may be applied to the microarrays to prevent non-specific binding by reactive groups that have not bound to a capture agent. Solutions of bovine serum albumin (BSA), casein, or nonfat milk, for example, may be used as blocking agents to reduce background binding in subsequent assays.

In preferred embodiments, high-precision, contact-printing robots are used to pick up small volumes of dissolved analytes from the wells of a microtiter plate and to repetitively deliver approximately 1 nL of the solutions to defined locations on the surfaces of substrates, such as chemically-derivatized glass microscope slides. Examples of such robots include the GMS 417 Arrayer, commercially available from Affymetrix of Santa Clara, Calif., and a split pin arrayer constructed according to instructions downloadable from the Brown lab website at http://cmgm.stanford.edu/pbrown. This results in the formation of microscopic spots of compounds on the slides. It will be appreciated by one of ordinary skill in the art, however, that the current invention is not limited to the delivery of 1 nL volumes of solution, to the use of particular robotic devices, or to the use of chemically derivatized glass slides, and that alternative means of delivery can be used that are capable of delivering picoliter or smaller volumes. Hence, in addition to a high precision array robot, other means for delivering the compounds can be used, including, but not limited to, ink jet printers, piezoelectric printers, and small volume pipetting robots.

In one embodiment, the compositions, e.g., microarrays or beads, comprising the analytes of the present invention may also comprise other components, e.g., molecules that recognize and bind specific peptides, metabolites, drugs or drug candidates, RNA, DNA, lipids, and the like. Thus, an array of analytes, only some of which bind a capture agent can comprise an embodiment of the invention.

As an alternative to planar microarrays, bead-based assays combined with fluorescence-activated cell sorting (FACS) have been developed to perform multiplexed immunoassays. Fluorescence-activated cell sorting has been routinely used in diagnostics for more than 20 years. Using mAbs, cell surface markers are identified on normal and neoplastic cell populations enabling the classification of various forms of leukemia or disease monitoring (recently reviewed by Herzenberg et al. Immunol Today 21 (2000), pp. 383-390).

Bead-based assay systems employ microspheres as solid support for the capture molecules instead of a planar substrate, which is conventionally used for microarray assays. In each individual immunoassay, the analyte is coupled to a distinct type of microsphere. The reaction takes place on the surface of the microspheres. The individual microspheres are color-coded by a uniform and distinct mixture of red and orange fluorescent dyes. After coupling to the appropriate analyte, the different color-coded bead sets can be pooled and the immunoassay is performed in a single reaction vial. Product formation of the analytes with their respective capture agents on the different bead types can be detected with a fluorescence-based reporter system. The signal intensities are measured in a flow cytometer, which is able to quantify the amount of captured targets on each individual bead. Each bead type and thus each immobilized target is identified using the color code measured by a second fluorescence signal. This allows the multiplexed quantification of multiple targets from a single sample. Sensitivity, reliability and accuracy are similar to those observed with standard microtiter ELISA procedures. Color-coded microspheres can be used to perform up to a hundred different assay types simultaneously (LabMAP system, Laboratory Multiple Analyte Profiling, Luminex, Austin, Tex., USA). For example, microsphere-based systems have been used to simultaneously quantify cytokines or autoantibodies from biological samples (Carson and Vignali, J Immunol Methods 227 (1999), pp. 41-52; Chen et al., Clin Chem 45 (1999), pp. 1693-1694; Fulton et al., Clin Chem 43 (1997), pp. 1749-1756). Bellisario et al. (Early Hum Dev 64 (2001), pp. 21-25) have used this technology to simultaneously measure antibodies to three HIV-1 antigens from newborn dried blood-spot specimens.

Bead-based systems have several advantages. As the small molecule analytes or PET sequences are coupled to distinct microspheres, each individual coupling event can be perfectly analyzed. Thus, only quality-controlled beads can be pooled for multiplexed immunoassays. Furthermore, if an additional parameter has to be included into the assay, one must only add a new type of loaded bead. No washing steps are required when performing the assay. The sample is incubated with the different bead types together with fluorescently labeled detection agents. After formation of the analyte-capture agent complex, only the fluorophores that are definitely bound to the surface of the microspheres are counted in the flow cytometer.

D. Exemplary Array Generation

The patent literature has reported a number of ways to generate peptide arrays, a few of which are represented below. All of them can be adapted for use in the instant invention, and are all incorporated herein by reference.

WO 03/038033A2 describes the use of ultrahigh resolution patterning, preferably carried out by dip-pen nanolithographic printing, for constructing peptide and protein nanoarrays with nanometer-level dimensions. The generated peptide and protein nanoarrays exhibit almost no detectable nonspecific binding of proteins to their passivated portions. This application demonstrates how dip pen nanolithographic printing can be used in methods to generate high density protein and peptide patterns, which exhibit bioactivity and virtually no non-specific adsorption. It also shows that one can use AFM-based screening procedures to study the reactivity of the features that comprise such nanoarrays. The method is suitable for a wide range of protein and peptide structures including peptides and antibodies. Features at or below 300 nm can be achieved using this method.

U.S. 20020037359A1 relates to arrays of peptidic molecules and the preparation of peptide arrays using focused acoustic energy. The arrays are prepared by acoustically ejecting peptide-containing fluid droplets from individual reservoirs towards designated sites on a substrate for attachment thereto.

One attempt at synthesizing a large number of diverse arrays of polypeptides and polymers in a smaller space is found in U.S. Pat. No. 5,143,854 granted to Pirrung et al. (1992). This patent describes the use of photo lithographic techniques for the solid phase synthesis of arrays of polypeptides and polymers. The disclosed technique uses “photomasks” and photo-labile protecting groups for protecting the underlying functional group. Each step of the process requires the use of a different photomask to control which regions are exposed to light and thus deprotected.

Another attempt to synthesize large numbers of polymers is disclosed by Southern in international patent application WO 93/22480, published Nov. 11, 1993. Southern describes a method for synthesizing polymers at selected sites by electrochemically modifying a surface—this method involves providing an electrolyte overlaying the surface and an array of electrodes adjacent to the surface. In each step of Southern's synthesis process, an array of electrodes is mechanically placed adjacent the points of synthesis, and a voltage is applied that is sufficient to produce electrochemical reagents at the electrode. The electrochemical reagents are deposited on the surface themselves or are allowed to react with another species, found either in the electrolyteor on the surface, in order to deposit or to modify a substance at the desired points of synthesis. The array of electrodes is then mechanically removed and the surface is subsequently contacted with selected monomers. For subsequent reactions, the array of electrodes is again mechanically placed adjacent the surface and a subsequent set of selected electrodes activated.

A more recent attempt to automate the synthesis of polymers is disclosed by Heller in international patent application WO 95/12808, published May 11, 1995. Heller describes aself-addressable, self-assembling microelectronic system that can carry out controlled multi-step reactions in microscopic environments, including biopolymer synthesis of oligonucleotides and peptides. The Heller method employs free field electrophoresis to transport analytes or reactants to selected micro-locations where they are effectively concentrated and reacted with the specific binding entities. Each micro-location of the Heller device has a derivatized surface for the covalent attachment of specific binding entities, which includes an attachment layer, a permeation layer, and an underlying direct current micro-electrode. The presence of the permeation layer prevents any electrochemically generated reagents from interacting with or binding to either the points of synthesis or to reagents that are electrophoretically transported to each synthesis site. Thus, all synthesis is due to reagents that are electrophoretically transported to each site of synthesis.

WO0053625A2 describes arrays designed to allow synthesizing chemical compounds such as peptides at well-defined and individually addressable locations. Such arrays may be manufactured at low cost by contracting fabricators using existing semiconductor manufacturing facilities. Briefly, the array may be coated with a biocompatible porous membrane that allows molecules to flow freely between a bulk solvent and an electrode. The array may then be immersed in a solution containing a precursor to an electrochemically-generated (ECG) reagent of interest. For peptide synthesis, this is preferably an ECG-reagent to remove amino protecting groups. A computer may then interface with the array to turn on the desired electrode pattern, and the precursor may be electrochemically converted into an active species. The electrochemically-generated (ECG) reagent, in turn, reacts with molecules immobilized to the membrane overlying the electrode.

A central feature of the preferred arrays according to the that technique is the ability to confine the ECG reagents to a region immediately adjacent to a selected microelectrode. Here, a fluorescein dye has been immobilized covalently at individually addressed microelectrode locations. The dye may be tightly confined to a checkerboard pattern and exhibits substantially no chemical cross-talk between active and inactive microelectrodes. This level of localization of ECG reagents may be achieved by exploiting the physical chemistry of the solution in which the microelectrode array is immersed. Such solutions usually contain buffers and scavengers that react with ECG reagents. However, the rate at which ECG reagents are produced can overwhelm the ability of the solution to react with them in the small local area immediately proximate to the microelectrode. As a result, chemistry that is mediated by ECG reagents occurs near selected microelectrodes, but there is no chemical cross-talk.

E. Exemplary Array Product

In a typical array construction with multiple reaction chambers, each chamber may contain up to 400 (20×20) spots of immobilized small molecules. Each of the spots may be about 200 micrometers in diameter, and is spaced at about 100 micrometers apart. Thus each chamber is about 6×6 mm in dimension. For accuracy, each peptide can be printed 4 or more times in each chamber, so that up to 100 peptides may be present in each chamber. Since the array may be used multiple times, the arrays may be used to simultaneously measure anywhere between 1-100 particular proteins in 4 samples. For positive control, each chamber may contain immobilized rabbit IgG, which will be bound by the labeled secondary agents. If less than 100 peptides are simultaneously measured, any of the unused immobilized analytes are negative controls for the analytes being measured.

If several of these arrays are used, the total number of proteins represented by these arrays may approach the total number of protein within a given proteome, or a specific subset thereof. Thus in another aspect, the invention provides compositions comprising a plurality of isolated and arrayed PET-containing peptides, wherein the PET-containing peptides represent at least 50%, 55%, 60%, 65%, 70%, 75%, 80%, 85%, 90% 95% or 100% of an organism's proteome, preferably serum proteome. In one embodiment, each of the PET-containing peptides is derived from a different protein. In another embodiment, the PET-containing peptides represents disease markers.

A packaged array product of the instant invention typically comprises an array of immobilized small molecules; a plurality of antibodies (or other capture agents) specific for these molecules in concentrated storage, one or more labeled secondary antibody, such as a fluorescent dye (e.g., Cy3) labeled or enzyme conjugated (e.g., HRP) antibody; appropriate washing buffers; chemical detection reagents (such as those for ECL) if necessary; an instruction including information regarding the immobilized peptides (identity, sequence, etc.), detailed assay parameters for each molecule, a standard competition curve for each molecule, a protocol for standard curve and sample measurement (including recommended dilution factors), and exemplary data processing procedures. For compatibility with other technology, the microarray slide can be manufactured in such a dimension so that it can be readily scanned with commercially available models of DNA microarray scanners, such as the GenePix 4000B scanner and the accompanying GenePix Pro software (Axon Instruments, Inc., Union City, Calif.).

V. Methods of Detecting Binding Events

In certain embodiments of the invention, there is a need to detect and quantitate the amount of capture agents bound to immobilized small molecules or PET peptides. Any of the following methods and other well-known methods in the art can be used to facilitate the detection/quantitation of binding.

In one embodiment, the capture agent or any secondary agent that can specifically bind the capture agent may be labeled with a detectable label, and the amount of bound label can then be directly measured. The term “label” is used herein in a broad sense to refer to agents that are capable of providing a detectable signal, either directly or through interaction with one or more additional members of a signal producing system. Labels that are directly detectable and may find use in the present invention include, for example, fluorescent labels such as fluorescein, rhodamine, BODIPY, cyanine dyes (e.g. from Amersham Pharmacia), Alexa dyes (e.g. from Molecular Probes, Inc.), fluorescent dye phosphoramidites, beads, chemilumninescent compounds, colloidal particles, and the like. Suitable fluorescent dyes are known in the art, including fluoresceinisothiocyanate (FITC); rhodamine and rhodamine derivatives; Texas Red; phycoerythrin; allophycocyanin; 6-carboxyfluorescein (6-FAM); 2′,7′-dimethoxy-41,51-dichloro carboxyfluorescein (JOE); 6-carboxy-X-rhodamine (ROX); 6-carboxy-21,41,71,4,7-hexachlorofluorescein (HEX); 5-carboxyfluorescein (5-FAM); N,N,N1,N′-tetramethyl carboxyrhodamine (TAMRA); sulfonated rhodamine; Cy3; Cy5, etc. Radioactive isotopes, such as ³⁵S, ³²P, ³H, ¹²⁵I, etc., and the like can also be used for labeling. In addition, labels may also include near-infrared dyes (Wang et al., Anal. Chem., 72:5907-5917 (2000), upconverting phosphors (Hampl et al., Anal. Biochem., 288:176-187 (2001), DNA dendrimers (Stears et al., Physiol. Genomics 3: 93-99 (2000), quantum dots (Bruchez et al., Science 281:2013-2016 (1998), latex beads (Okana et al., Anal. Biochem. 202:120-125 (1992), selenium particles (Stimpson et al., Proc. Natl. Acad. Sci. 92:6379-6383 (1995), and europium nanoparticles (Harma et al., Clin. Chem. 47:561-568 (2001). The label is one that preferably does not provide a variable signal, but instead provides a constant and reproducible signal over a given period of time.

Here below describes a simple calculation of the optimum concentration of labeled antigen to use for achieving better dynamic range. The same calculation can be adopted to calculate the optimum concentration of labeled capture agents to achieve better dynamic range in the array-based competition assay.

Generally, in a parallel, competitive immunoassay, an array of antibodies on a fixed support is used to quantitate the amount of a set of antigens in solution. This can be done by introducing a known quantity of labeled antigen into the sample, quantitating the amount of labeled antigen-antibody complex formed at equilibrium, and then calculating the amount of unlabeled antigen in the original sample. One difficulty that arises with such parallel arrays is that the range of antibody-antigen concentrations that can be measured in a single detection scan may be limited, for example, to 2 or 3 orders of magnitude, by the specific detection scheme. It may be desirable to control the amount of each labeled antigen such that the amount of labeled antigen-antibody complex is within this 2 to 3 order of magnitude range for each complex. To do this, one must have fairly good knowledge of each antibody affinity and the concentration of each “unknown” antigen to be quantitated. The appropriate amount of labeled antigen to add to the analyte can then be computed via the approach discussed below.

Each antibody-antigen pair reacts to form a complex, represented schematically by:
A+B [symbol] AB (1)
A+B* [symbol] AB*(2)
where A, B, B*, AB, and AB* represent the antibody, the unlabeled antigen, the labeled antigen, the unlabeled complex, and the labeled complex, respectively. When the array and the sample are contacted and allowed to reach equilibrium, the concentration of the species above are related by:
   K_{d}[AB]=[A][B] (3)
   K_{d}[AB*]=[A][B*] (4)
where K_{d} is the equilibrium dissociation constant (presumably the same as the solution-phase reaction of A and B), and [ ] denotes the concentration of the species enclosed in brackets. The concentrations of the surface species, [A], [AB], and [AB*], are computed as the number of moles of each species on the array divided by the analyte volume. Using the initial conditions, [A]₀, [B]₀, and [B*]₀, the unknowns [A] and [B] can be eliminated:
   K_{d}[AB]=([A]₀−[AB]−[AB*])([B]₀−[AB]) (5)
   K_{d}[AB*]=([A]₀−[AB]−[AB*])([B*]₀−[AB*]) (6)

Assuming K_{d}, [A]₀, and [B*]₀ are known and [AB*] will be measured by the assay, the above two equations contain two unknowns, [AB] and [B]₀. Solving for [B]₀ leads to:
[B]₀={[B*]₀(−K_{d}[AB*]+[A]₀[B*]₀−[A]₀[AB*]−[AB*][B*]₀+[AB*]²)}/[AB*]([B*]₀−[AB*]) (7)
which is one way to calculate the unknown concentration of antigen in the sample. In a microarray format, it is very likely, in fact desirable, that the extent of antigen binding has a negligible effect on the antigen concentration in solution ([B]≅[B]₀ and [B*]≅[B*]₀), leading to the simpler form:
   [B]₀=[B*]₀([A]₀/[AB*]−1)−K_{d} (8)

Some numerical examples are shown in the Table below.

Example calculations of antigen concentration in the sample [B]₀ via equation 7

| K_{d} (nM) | [A]₀ (fM) | [B*]₀ (nM) | [AB*] (fM) | [B]₀ (nM) |
|---|---|---|---|---|
| 10 | 1 | 100 | 0.5 | 90 |
| 1 | 1 | 100 | 0.5 | 99 |
| 0.1 | 1 | 100 | 0.5 | 99.9 |
| 1 | 1 | 100 | 0.5 | 9 |
| 0.1 | 1 | 1 | 0.5 | 0.9 |

The approximation leading to equation 8 from equation 7 is good to 4-7 digits in [B]₀.

The difficulty with this approach in a parallel array is that the range of [AB*] that can be measured may be much smaller than its actual range. For example, on an array of spots each containing 10⁶ molecules (about 1 pg of 150 kD antibody), the range of [AB*] can be 6 logs (from 1 to 10⁶). The detector's range may be significantly less than this, perhaps 2-3 logs. Thus, the range of values from a single detector scan will only be 2-3 logs. One way to circumvent this problem is to adjust the concentration of labeled antigen ([B*]) by pre-binding some antigen (or any other method which leads to a controlled fraction of antigen bound to the array being (un)labeled at equilibrium). In this case, we would like to know what [B*] should be used for each antigen given a target [AB*] and estimates of K_{d}, [A]₀, and [B]₀ for that antigen. This can be accomplished by solving equation 8 for the required [B*]₀:
[B*]₀=([B]₀+K_{d})/{[A]₀/[AB*]−1} (9)
where [A]₀/[AB*] is the variable that it will be desirable to hold relatively constant, for example around 1000 (106 molecules half bound on a log scale). The required amount of labeled antigen is therefore proportional to [B]₀ when [B]₀>>K_{d}, and constant when [B]₀<<K_{d}.

A very useful labeling agent is water-soluble quantum dots, or so-called “functionalized nanocrystals” or “semiconductor nanocrystals” as described in U.S. Pat. No. 6,114,038. Generally, quantum dots can be prepared which result in relative monodispersity (e.g., the diameter of the core varying approximately less than 10% between quantum dots in the preparation), as has been described previously (Bawendi et al., 1993, J. Am. Chem. Soc. 115:8706). Examples of quantum dots are known in the art to have a core selected from the group consisting of CdSe, CdS, and CdTe (collectively referred to as “CdX”)(see, e.g., Norris et al., 1996, Physical Review B. 53:16338-16346; Nirmal et al., 1996, Nature 383:802-804; Empedocles et al., 1996, Physical Review Letters 77:3873-3876; Murray et al., 1996, Science 270: 1355-1338; Effros et al., 1996, Physical Review B. 54:4843-4856; Sacra et al., 1996, J. Chem. Phys. 103:5236-5245; Murakoshi et al., 1998, J. Colloid Interface Sci. 203:225-228; Optical Materials and Engineering News, 1995, Vol. 5, No. 12; and Murray et al., 1993, J. Am. Chem. Soc. 115:8706-8714; the disclosures of which are hereby incorporated by reference).

CdX quantum dots have been passivated with an inorganic coating (“shell”) uniformly deposited thereon. Passivating the surface of the core quantum dot can result in an increase in the quantum yield of the luminescence emission, depending on the nature of the inorganic coating. The shell which is used to passivate the quantum dot is preferably comprised of YZ wherein Y is Cd or Zn, and Z is S, or Se. Quantum dots having a CdX core and a YZ shell have been described in the art (see, e.g., Danek et al., 1996, Chem. Mater. 8:173-179; Dabbousi et al., 1997, J. Phys. Chem. B 101:9463; Rodriguez-Viejo et al., 1997, Appl. Phys. Lett. 70:2132-2134; Peng et al., 1997, J. Am. Chem. Soc. 119:7019-7029; 1996, Phys. Review B. 53:16338-16346; the disclosures of which are hereby incorporated by reference). However, the above described quantum dots, passivated using an inorganic shell, have only been soluble in organic, non-polar (or weakly polar) solvents. To make quantum dots useful in biological applications, it is desirable that the quantum dots are water-soluble. “Water-soluble” is used herein to mean sufficiently soluble or suspendable in an aqueous-based solution, such as in water or water-based solutions or buffer solutions, including those used in biological or molecular detection systems as known by those skilled in the art.

U.S. Pat. No. 6,114,038 provides a composition comprising functionalized nanocrystals for use in non-isotopic detection systems. The composition comprises quantum dots (capped with a layer of a capping compound) that are water-soluble and functionalized by operably linking, in a successive manner, one or more additional compounds. In a preferred embodiment, the one or more additional compounds form successive layers over the nanocrystal. More particularly, the functionalized nanocrystals comprise quantum dots capped with the capping compound, and have at least a diaminocarboxylic acid which is operatively linked to the capping compound. Thus, the functionalized nanocrystals may have a first layer comprising the capping compound, and a second layer comprising a diaminocarboxylic acid; and may further comprise one or more successive layers including a layer of amino acid, a layer of affinity ligand, or multiple layers comprising a combination thereof. The composition comprises a class of quantum dots that can be excited with a single wavelength of light resulting in detectable luminescence emissions of high quantum yield and with discrete luminescence peaks. Such functionalized nanocrystal may be used to label capture agents or secondary agents of the instant invention for their use in the detection and/or quantitafion of the binding events.

U.S. Pat. No. 6,326,144 describes quantum dots (QDs) having a characteristic spectral emission, which is tunable to a desired energy by selection of the particle size of the quantum dot. For example, a 2 nanometer quantum dot emits green light, while a 5 nanometer quantum dot emits red light. The emission spectra of quantum dots have linewidths as narrow as 25-30 nm depending on the size heterogeneity of the sample, and lineshapes that are symmetric, gaussian or nearly gaussian with an absence of a tailing region. The combination of tunability, narrow linewidths, and symmetric emission spectra without a tailing region provides for high resolution of multiply-sized quantum dots within a system and enables researchers to examine simultaneously a variety of biological moieties tagged with QDs. In addition, the range of excitation wavelengths of the nanocrystal quantum dots is broad and can be higher in energy than the emission wavelengths of all available quantum dots. Consequently, this allows the simultaneous excitation of all quantum dots in a system with a single light source, usually in the ultraviolet or blue region of the spectrum. QDs are also more robust than conventional organic fluorescent dyes and are more resistant to photobleaching than the organic dyes. The robustness of the QD also alleviates the problem of contamination of the degradation products of the organic dyes in the system being examined. These QDs can be used for labeling capture agents of protein, nucleic acid, and other biological molecules in nature. Cadmium Selenide quantum dot nanocrystals are available from Quantum Dot Corporation of Hayward, Calif.

Alternatively, the primary capture agent is not labeled, but a secondary labeled reagent specific for the capture agent is added in order to detect the presence or quantitate the amount of primary capture agent on the immobilized PET-peptide fragments. This method of detection have the disadvantage that two reagents (the primary capture agent and the secondary agent) must be developed for each protein, one to capture/bind the PET and one to label the capture agent once bound. Such methods have the advantage that they are characterized by an inherently improved signal to noise ratio as they exploit two binding reactions, thus the presence and/or concentration of the protein can be measured with more accuracy and precision because of the increased signal to noise ratio.

In yet another embodiment, the subject peptide array can be a “virtual arrays”. For example, a virtual array can be generated in which PET-containing peptides are immobilized on beads whose identity, with respect to the particular PET it is specific for as a consequence to the associated capture agent, is encoded by a particular ratio of two or more covalently attached dyes. Mixtures of encoded PET-beads are added to a sample, resulting in binding of the capture agents to the PET entities, at the presence or absence of different concentrations of competition peptide fragments.

To quantitate the captured agents remaining bound, the beads are then introduced into an instrument, such as a flow cytometer, that reads the intensity of the various fluorescence signals on each bead, and the identity of the bead can be determined by measuring the ratio of the dyes. This technology is relatively fast and efficient, and can be adapted by researchers to monitor almost any PET of interest.

Preferably, the capture agent to be labeled is combined with an activated dye that reacts with a group present on the capture agent, e.g., amine groups, thiol groups, or aldehyde groups.

The label may also be a covalently bound enzyme capable of providing a detectable product signal after addition of suitable substrate. Examples of suitable enzymes for use in the present invention include horseradish peroxidase, alkaline phosphatase, malate dehydrogenase and the like.

Enzyme-Linked Immunosorbent Assay (ELISA) may also be used for detection of a protein that interacts with a capture agent. In an ELISA, the indicator molecule is covalently coupled to an enzyme and may be quantified by determining with a spectrophotometer the initial rate at which the enzyme converts a clear substrate to a correlated product. Methods for performing ELISA are well known in the art and described in, for example, Perlmann, H. and Perlmann, P. (1994). Enzyme-Linked Immunosorbent Assay. In: Cell Biology: A Laboratory Handbook. San Diego, Calif., Academic Press, Inc., 322-328; Crowther, J. R. (1995). Methods in Molecular Biology, Vol. 42-ELISA: Theory and Practice. Humana Press, Totowa, N.J.; and Harlow, E. and Lane, D. (1988). Antibodies: A Laboratory Manual. Cold Spring Harbor Laboratory Press, Cold Spring Harbor, N.Y., 553-612, the contents of each of which are incorporated by reference.

A fully-automated, microarray-based approach for high-throughput, ELISAs was described by Mendoza et al. (BioTechniques 27:778-780,782-786,788, 1999). This system consisted of an optically flat glass plate with 96 wells separated by a Teflon mask. More than a hundred peptides can be immobilized in each well. Sample incubation, washing and fluorescence-based detection were performed with an automated liquid pipettor. The microarrays were quantitatively imaged with a scanning charge-coupled device (CCD) detector. Thus, the feasibility of multiplex detection of arrayed antigens in a high-throughput fashion using marker antigens could be successfully demonstrated. In addition, Silzel et al. (Clin Chem 44 pp. 2036-2043, 1998) could demonstrate that multiple IgG subclasses can be detected simultaneously using microarray technology. Wiese et al. (Clin Chem 47 pp. 1451-1457, 2001) were able to measure prostate-specific antigen (PSA), —(1)-antichymotrypsin-bound PSA and interleukin-6 in a microarray format. Arenkov et al. (supra) carried out microarray sandwich immunoassays and direct antigen or antibody detection experiments using a modified polyacrylamide gel as substrate for immobilized capture molecules.

Most of the microarray assay formats described in the art rely on chemiluminescence- or fluorescence-based detection methods. A further improvement with regard to sensitivity involves the application of fluorescent labels and waveguide technology. A fluorescence-based array immunosensor was developed by Rowe et al. (Anal Chem 71 (1999), pp. 433-439; and Biosens Bioelectron 15 (2000), pp. 579-589) and applied for the simultaneous detection of clinical analytes using the sandwich immunoassay format. Biotinylated PET peptides can be immobilized on avidin-coated waveguides using a flow-chamber module system. Discrete regions of PET peptides can be vertically arranged on the surface of the waveguide. Samples of interest, including capture agents and competition peptides, can be incubated to allow the capture molecules to bind to their PET-peptides. Bound capture agents are then visualized with appropriate fluorescently labeled detection molecules. This type of array immunosensor was shown to be appropriate for the detection and measurement of targets at physiologically relevant concentrations in a variety of clinical samples.

A further increase in the sensitivity using waveguide technology was achieved with the development of the planar waveguide technology (Duveneck et al., Sens Actuators B B38 (1997), pp. 88-95). Thin-film waveguides are generated from a high-refractive material such as Ta₂O₅ that is deposited on a transparent substrate. Laser light of desired wavelength is coupled to the planar waveguide by means of diffractive grating. The light propagates in the planar waveguide and an area of more than a square centimeter can be homogeneously illuminated. At the surface, the propagating light generates a so-called evanescent field. This extends into the solution and activates only fluorophores that are bound to the surface. Fluorophores in the surrounding solution are not excited. Close to the surface, the excitation field intensities can be a hundred times higher than those achieved with standard confocal excitation. A CCD camera is used to identify signals simultaneously across the entire area of the planar waveguide. Thus, the immobilization of the PET peptides in a microarray format on the planar waveguide allows the performance of highly sensitive miniaturized and parallelized immunoassays. This type of system was successfully employed to detect interleukin-6 at concentrations as low as 40 fM and has the additional advantage that the assay can be performed without washing steps that are usually required to remove unbound detection molecules (Weinberger et al., Pharmacogenomics 1 (2000), pp. 395-416).

Alternative strategies pursued to increase sensitivity are based on signal amplification procedures. For example, immunoRCA (immuno rolling circle amplification) involves an oligonucleotide primer that is covalently attached to a detection molecule (such as a second capture agent in a sandwich-type assay format). Using circular DNA as template, which is complementary to the attached oligonucleotide, DNA polymerase will extend the attached oligonucleotide and generate a long DNA molecule consisting of hundreds of copies of the circular DNA, which remains attached to the detection molecule. The incorporation of thousands of fluorescently labeled nucleotides will generate a strong signal. Schweitzer et al. (Proc Natl Acad Sci USA 97 (2000), pp. 10113-10119) have evaluated this detection technology for use in microarray-based assays. Sandwich immunoassays for hulgE and prostate-specific antigens were performed in a microarray format. The antigens could be detected at femtomolar concentrations and it was possible to score single, specifically captured antigens by counting discrete fluorescent signals that arose from the individual antibody-antigen complexes. The authors demonstrated that immunoassays employing rolling circle DNA amplification are a versatile platform for the ultra-sensitive detection of antigens and thus are well suited for use in protein microarray technology.

A novel technology for protein detection, proximity ligation, has recently been developed, along with improved methods for in situ synthesis of DNA microarrays. Proximity ligation may be another amplification strategy that can be employed with anti-PET antibodies. Proximity ligation enables a specific and quantitative transformation of proteins present in a sample into nucleic acid sequences. As pairs of so-called proximity probes bind the individual target molecules at distinct sites (say two adjacent epitopes on the same target molecule), these proximity probes are brought in close proximity. The probes consist of a protein specific binding part coupled to an oligonucleotide with either a free 3′- or 5′-end capable of hybridizing to a common connector oligonucleotide. When the probes are in proximity, promoted by target binding, the polynucleotide strands can be joined by enzymatic ligation. The nucleic acid sequence that is formed can then be amplified and quantitatively detected in a real-time monitored polymerase chain reaction or any type of polynucleotide amplification method (such as rolling circle amplification, etc.). In certain embodiments, the common connector oligonucleotide may be omitted, and the ends of the oligonucleotides on the proximity probes may be directly ligated by, for example, T4 DNA ligase. This convenient assay is simple to perform and allows highly sensitive protein detection. It also eliminates or significantly reduces background issue associated with the immuno-PCR method (Sano et al., Chemtech January 1995, pp 24-30), where non-specifically bound oligonucleotides may also be accidentally amplified by the very sensitive PCR method. See WO 97/00446, WO 01/61037 and WO 03/044231, entire contents of which are all incorporated herein by reference.

In certain embodiments, immuno-PCR method such as those described in Sano et al., Chemtech January 1995, pp 24-30 (incorporated herein by reference) may be used to detect any capture agents (e.g. Ab) that specifically bind the immobilized target analytes.

Radioimmunoassays (RIA) may also be used for detection of a protein that interacts with a capture agent. In a RIA, the indicator molecule is labeled with a radioisotope and it may be quantified by counting radioactive decay events in a scintillation counter. Methods for performing direct or competitive RIA are well known in the art and described in, for example, Cell Biology: A Laboratory Handbook. San Diego, Calif., Academic Press, Inc., the contents of which are incorporated herein by reference.

Other immunoassays commonly used to quantitate the levels of proteins in cell samples, and are well-known in the art, can be adapted for use in the instant invention. The invention is not limited to a particular assay procedure, and therefore is intended to include both homogeneous and heterogeneous procedures. Exemplary other immunoassays which can be conducted according to the invention include fluorescence polarization immunoassay (FPIA), fluorescence immunoassay (FIA), enzyme immunoassay (EIA), nephelometric inhibition immunoassay (NIA). An indicator moiety, or label group, can be attached to the subject antibodies and is selected so as to meet the needs of various uses of the method which are often dictated by the availability of assay equipment and compatible immunoassay procedures. General techniques to be used in performing the various immunoassays noted above are known to those of ordinary skill in the art. In one embodiment, the determination of protein level in a biological sample may be performed by a microarray analysis (protein chip).

In several other embodiments, detection of the presence of a protein that interacts with a capture agent may be achieved without labeling. For example, determining the ability of a protein to bind to a capture agent can be accomplished using a technology such as real-time Biomolecular Interaction Analysis (BIA). Sjolander, S. and Urbaniczky, C. (1991) Anal. Chem. 63:2338-2345 and Szabo et al. (1995) Curr. Opin. Struct. Biol. 5:699-705. As used herein, “BIA” is a technology for studying biospecific interactions in real time, without labeling any of the interactants (e.g., BIAcore).

In another embodiment, a biosensor with a special diffractive grating surface may be used to detect/quantitate binding between PET-containing peptides immobilized at the surface of the biosensor and non-labeled capture agents. Details of the technology is described in more detail in B. Cunningham, P. Li, B. Lin, J. Pepper, “Colorimetric resonant reflection as a direct biochemical assay technique,” Sensors and Actuators B, Volume 81, p. 316-328, Jan 5, 2002, and in PCT No. WO 02/061429 A2 and U.S. 2003/0032039. Briefly, a guided mode resonant phenomenon is used to produce an optical structure that, when illuminated with collimated white light, is designed to reflect only a single wavelength (color). When molecules are attached to the surface of the biosensor, the reflected wavelength (color) is shifted due to the change of the optical path of light that is coupled into the grating. By linking molecules to the grating surface, complementary binding molecules can be detected/quantitated without the use of any kind of fluorescent probe or particle label. The spectral shifts may be analyzed to determine the expression data provided, and to indicate the presence or absence of a particular indication.

The biosensor typically comprises: a two-dimensional grating comprised of a material having a high refractive index, a substrate layer that supports the two-dimensional grating, and one or more detection probes immobilized on the surface of the two-dimensional grating opposite of the substrate layer. When the biosensor is illuminated a resonant grating effect is produced on the reflected radiation spectrum. The depth and period of the two-dimensional grating are less than the wavelength of the resonant grating effect.

A narrow band of optical wavelengths can be reflected from the biosensor when it is illuminated with a broad band of optical wavelengths. The substrate can comprise glass, plastic or epoxy. The two-dimensional grating can comprise a material selected from the group consisting of zinc sulfide, titanium dioxide, tantalum oxide, and silicon nitride.

The substrate and two-dimensional grating can optionally comprise a single unit. The surface of the single unit comprising the two-dimensional grating is coated with a material having a high refractive index, and the one or more detection probes are immobilized on the surface of the material having a high refractive index opposite of the single unit. The single unit can be comprised of a material selected from the group consisting of glass, plastic, and epoxy.

The biosensor can optionally comprise a cover layer on the surface of the two-dimensional grating opposite of the substrate layer. The one or more detection probes are immobilized on the surface of the cover layer opposite of the two-dimensional grating. The cover layer can comprise a material that has a lower refractive index than the high refractive index material of the two-dimensional grating. For example, a cover layer can comprise glass, epoxy, and plastic.

A two-dimensional grating can be comprised of a repeating pattern of shapes selected from the group consisting of lines, squares, circles, ellipses, triangles, trapezoids, sinusoidal waves, ovals, rectangles, and hexagons. The repeating pattern of shapes can be arranged in a linear grid, i.e., a grid of parallel lines, a rectangular grid, or a hexagonal grid. The two-dimensional grating can have a period of about 0.01 microns to about 1 micron and a depth of about 0.01 microns to about 1 micron.

To illustrate, biochemical interactions occurring on a surface of a calorimetric resonant optical biosensor embedded into a surface of a microarray slide, microtiter plate or other device, can be directly detected and measured on the sensor's surface without the use of fluorescent tags or calorimetric labels. The sensor surface contains an optical structure that, when illuminated with collimated white light, is designed to reflect only a narrow band of wavelengths (color). The narrow wavelength is described as a wavelength “peak.” The “peak wavelength value” (PWV) changes when biological material is deposited or removed from the sensor surface, such as when binding occurs. Such binding-induced change of PWV can be measured using a measurement instrument disclosed in U.S. 2003/0032039.

In one embodiment, the instrument illuminates the biosensor surface by directing a collimated white light on to the sensor structure. The illuminated light may take the form of a spot of collimated light. Alternatively, the light is generated in the form of a fan beam. The instrument collects light reflected from the illuminated biosensor surface. The instrument may gather this reflected light from multiple locations on the biosensor surface simultaneously. The instrument can include a plurality of illumination probes that direct the light to a discrete number of positions across the biosensor surface. The instrument measures the Peak Wavelength Values (PWVs) of separate locations within the biosensor-embedded microtiter plate using a spectrometer. In one embodiment, the spectrometer is a single-point spectrometer. Alternatively, an imaging spectrometer is used. The spectrometer can produce a PWV image map of the sensor surface. In one embodiment, the measuring instrument spatially resolves PWV images with less than 200 micron resolution.

In one embodiment, a subwavelength structured surface (SWS) may be used to create a sharp optical resonant reflection at a particular wavelength that can be used to track with high sensitivity the interaction of biological materials, such as specific binding substances or binding partners or both. A calorimetric resonant diffractive grating surface acts as a surface binding platform for specific binding substances (such as immobilized PET-peptides of the instant invention). SWS is an unconventional type of diffractive optic that can mimic the effect of thin-film coatings. (Peng & Morris, “Resonant scattering from two-dimensional gratings,” J. Opt. Soc. Am. A, Vol. 13, No. 5, p. 993, May; Magnusson, & Wang, “New principle for optical filters,” Appl. Phys. Lett., 61, No. 9, p. 1022, August, 1992; Peng & Morris, “Experimental demonstration of resonant anomalies in diffraction from two-dimensional gratings,” Optics Letters, Vol. 21, No. 8, p. 549, April, 1996). A SWS structure contains a surface-relief, two-dimensional grating in which the grating period is small compared to the wavelength of incident light so that no diffractive orders other than the reflected and transmitted zeroth orders are allowed to propagate. A SWS surface narrowband filter can comprise a two-dimensional grating sandwiched between a substrate layer and a cover layer that fills the grating grooves. Optionally, a cover layer is not used. When the effective index of refraction of the grating region is greater than the substrate or the cover layer, a waveguide is created. When a filter is designed accordingly, incident light passes into the waveguide region. A two-dimensional grating structure selectively couples light at a narrow band of wavelengths into the waveguide. The light propagates only a short distance (on the order of 10-100 micrometers), undergoes scattering, and couples with the forward- and backward-propagating zeroth-order light. This sensitive coupling condition can produce a resonant grating effect on the reflected radiation spectrum, resulting in a narrow band of reflected or transmitted wavelengths (colors). The depth and period of the two-dimensional grating are less than the wavelength of the resonant grating effect.

The reflected or transmitted color of this structure can be modulated by the addition of molecules such as capture agents with or without the competition peptides, to the upper surface of the cover layer or the two-dimensional grating surface. The added molecules increase the optical path length of incident radiation through the structure, and thus modify the wavelength (color) at which maximum reflectance or transmittance will occur. Thus in one embodiment, a biosensor, when illuminated with white light, is designed to reflect only a single wavelength. When specific binding substances are attached to the surface of the biosensor, the reflected wavelength (color) is shifted due to the change of the optical path of light that is coupled into the grating. By linking specific binding substances to a biosensor surface, complementary binding partner molecules can be detected without the use of any kind of fluorescent probe or particle label. The detection technique is capable of resolving changes of, for example, about 0.1 nm thickness of protein binding, and can be performed with the biosensor surface either immersed in fluid or dried. This PWV change can be detected by a detection system consists of, for example, a light source that illuminates a small spot of a biosensor at normal incidence through, for example, a fiber optic probe. A spectrometer collects the reflected light through, for example, a second fiber optic probe also at normal incidence. Because no physical contact occurs between the excitation/detection system and the biosensor surface, no special coupling prisms are required. The biosensor can, therefore, be adapted to a commonly used assay platform including, for example, microtiter plates and microarray slides. A spectrometer reading can be performed in several milliseconds, thus it is possible to efficiently measure a large number of molecular interactions taking place in parallel upon a biosensor surface, and to monitor reaction kinetics in real time.

Various embodiments, variations of the biosensor described above can be found in U.S. 2003/0032039, incorporated herein by reference in its entirety.

One or more specific analytes may be immobilized on the two-dimensional grating or cover layer, if present. Immobilization may occur by any of the above described methods. Suitable capture agents can be, for example, a nucleic acid, polypeptide, antigen, polyclonal antibody, monoclonal antibody, single chain antibody (scFv), F(ab) fragment, F(ab′)₂ fragment, Fv fragment, small organic molecule, even cell, virus, or bacteria. A biological sample can be obtained and/or derived from, for example, blood, plasma, serum, gastrointestinal secretions, homogenates of tissues or tumors, synovial fluid, feces, saliva, sputum, cyst fluid, amniotic fluid, cerebrospinal fluid, peritoneal fluid, lung lavage fluid, semen, lymphatic fluid, tears, or prostatitc fluid. Preferably, one or more specific analytes are arranged in a microarray of distinct locations on a biosensor. A microarray of analytes comprises one or more specific analytes on a surface of a biosensor such that a biosensor surface contains a plurality of distinct locations, each with a different analyte or with a different amount of a specific analyte. For example, an array can comprise 1, 10, 100, 1,000, 10,000, or 100,000 distinct locations. A biosensor surface with a large number of distinct locations is called a microarray because one or more specific analytes are typically laid out in a regular grid pattern in x-y coordinates. However, a microarray can comprise one or more specific analytes laid out in a regular or irregular pattern.

A microarray spot can range from about 50 to about 500 microns in diameter. Alternatively, a microarray spot can range from about 150 to about 200 microns in diameter. One or more specific analytes can be bound to their specific capture agents, at the presence or absence of the competition peptides.

In one biosensor embodiment, a microarray on a biosensor is created by placing microdroplets of one or more specific analytes onto, for example, an x-y grid of locations on a two-dimensional grating or cover layer surface. When the biosensor is exposed to a test sample comprising capture agents and competition peptides, the binding partners will be preferentially attracted to distinct locations on the microarray that comprise capture agents that have high affinity for the analyte binding partners. Some of the distinct locations will gather binding partners onto their surface, while other locations will not. Thus a specific capture agent specifically binds to its immobilized analyte binding partner, but does not substantially bind other analyte binding partners on the biosensor. By application of specific analytes with a microarray spotter onto a biosensor, specific binding substance densities of 10,000 specific binding substances/in² can be obtained. By focusing an illumination beam of a fiber optic probe to interrogate a single microarray location, a biosensor can be used as a label-free microarray readout system.

For the detection of analytes at concentrations of less than about 0.1 ng/ml, one may amplify and transduce binding partners bound to a biosensor into an additional layer on the biosensor surface. The increased mass deposited on the biosensor can be detected as a consequence of increased optical path length. By incorporating greater mass onto a biosensor surface, an optical density of binding partners on the surface is also increased, thus rendering a greater resonant wavelength shift than would occur without the added mass. The addition of mass can be accomplished, for example, enzymatically, through a “sandwich” assay, or by direct application of mass (such as a second capture agent specific for the first capture agent) to the biosensor surface in the form of appropriately conjugated beads or polymers of various size and composition. This principle has been exploited for other types of optical biosensors to demonstrate sensitivity increases over 1500× beyond sensitivity limits achieved without mass amplification. See, e.g., Jenison et al., “Interference-based detection of nucleic acid targets on optically coated silicon,” Nature Biotechnology, 19: 62-65, 2001.

In an alternative embodiment, a biosensor comprises volume surface-relief volume diffractive structures (a SRVD biosensor). SRVD biosensors have a surface that reflects predominantly at a particular narrow band of optical wavelengths when illuminated with a broad band of optical wavelengths. Where specific capture agents and/or analytes are immobilized on a SRVD biosensor, the reflected wavelength of light is shifted. One-dimensional surfaces, such as thin film interference filters and Bragg reflectors, can select a narrow range of reflected or transmitted wavelengths from a broadband excitation source. However, the deposition of additional material, such as specific capture agents and/or analytes onto their upper surface results only in a change in the resonance linewidth, rather than the resonance wavelength. In contrast, SRVD biosensors have the ability to alter the reflected wavelength with the addition of material, such as specific capture agents and/or binding partners to the surface.

A SRVD biosensor comprises a sheet material having a first and second surface. The first surface of the sheet material defines relief volume diffraction structures. Sheet material can comprise, for example, plastic, glass, semiconductor wafer, or metal film. A relief volume diffractive structure can be, for example, a two-dimensional grating, as described above, or a three-dimensional surface-relief volume diffractive grating. The depth and period of relief volume diffraction structures are less than the resonance wavelength of light reflected from a biosensor. A three-dimensional surface-relief volume diffractive grating can be, for example, a three-dimensional phase-quantized terraced surface relief pattern whose groove pattern resembles a stepped pyramid. When such a grating is illuminated by a beam of broadband radiation, light will be coherently reflected from the equally spaced terraces at a wavelength given by twice the step spacing times the index of refraction of the surrounding medium. Light of a given wavelength is resonantly diffracted or reflected from the steps that are a half-wavelength apart, and with a bandwidth that is inversely proportional to the number of steps. The reflected or diffracted color can be controlled by the deposition of a dielectric layer so that a new wavelength is selected, depending on the index of refraction of the coating.

A stepped-phase structure can be produced first in photoresist by coherently exposing a thin photoresist film to three laser beams, as described previously. See e.g., Cowen, “The recording and large scale replication of crossed holographic grating arrays using multiple beam interferometry,” in International Conference on the Application, Theory, and Fabrication of Periodic Structures, Diffraction Gratings, and Moire Phenomena II, Lerner, ed., Proc. Soc. Photo-Opt. Instrum. Eng., 503, 120-129, 1984; Cowen, “Holographic honeycomb microlens,” Opt. Eng. 24, 796-802 (1985); Cowen & Slafer, “The recording and replication of holographic micropatterns for the ordering of photographic emulsion grains in film systems,” J Imaging Sci. 31, 100-107, 1987. The nonlinear etching characteristics of photoresist are used to develop the exposed film to create a three-dimensional relief pattern. The photoresist structure is then replicated using standard embossing procedures. For example, a thin silver film may be deposited over the photoresist structure to form a conducting layer upon which a thick film of nickel can be electroplated. The nickel “master” plate is then used to emboss directly into a plastic film, such as vinyl, that has been softened by heating or solvent. A theory describing the design and fabrication of three-dimensional phase-quantized terraced surface relief pattern that resemble stepped pyramids is described: Cowen, “Aztec surface-relief volume diffractive structure,” J. Opt. Soc. Am. A, 7:1529 (1990). An example of a three-dimensional phase-quantized terraced surface relief pattern may be a pattern that resembles a stepped pyramid. Each inverted pyramid is approximately 1 micron in diameter. Preferably, each inverted pyramid can be about 0.5 to about 5 microns diameter, including for example, about 1 micron. The pyramid structures can be close-packed so that a typical microarray spot with a diameter of 150-200 microns can incorporate several hundred stepped pyramid structures. The relief volume diffraction structures have a period of about 0.1 to about 1 micron and a depth of about 0.1 to about 1 micron.

One or more specific binding substances, as described above, are immobilized on the reflective material of a SRVD biosensor. One or more specific binding substances can be arranged in microarray of distinct locations, as described above, on the reflective material.

A SRVD biosensor reflects light predominantly at a first single optical wavelength when illuminated with a broad band of optical wavelengths, and reflects light at a second single optical wavelength when one or more specific binding substances are immobilized on the reflective surface. The reflection at the second optical wavelength results from optical interference. A SRVD biosensor also reflects light at a third single optical wavelength when the one or more specific capture agents are bound to their respective analytes, due to optical interference. Readout of the reflected color can be performed serially by focusing a microscope objective onto individual microarray spots and reading the reflected spectrum with the aid of a spectrograph or imaging spectrometer, or in parallel by, for example, projecting the reflected image of the microarray onto an imaging spectrometer incorporating a high resolution color CCD camera.

A SRVD biosensor can be manufactured by, for example, producing a metal master plate, and stamping a relief volume diffractive structure into, for example, a plastic material like vinyl. After stamping, the surface is made reflective by blanket deposition of, for example, a thin metal film such as gold, silver, or aluminum. Compared to MEMS-based biosensors that rely upon photolithography, etching, and wafer bonding procedures, the manufacture of a SRVD biosensor is very inexpensive.

A SWS or SRVD biosensor embodiment can comprise an inner surface. In one preferred embodiment, such an inner surface is a bottom surface of a liquid-containing vessel. A liquid-containing vessel can be, for example, a microtiter plate well, a test tube, a petri dish, or a microfluidic channel. In one embodiment, a SWS or SRVD biosensor is incorporated into a microtiter plate. For example, a SWS biosensor or SRVD biosensor can be incorporated into the bottom surface of a microtiter plate by assembling the walls of the reaction vessels over the resonant reflection surface, so that each reaction “spot” can be exposed to a distinct test sample. Therefore, each individual microtiter plate well can act as a separate reaction vessel. Separate chemical reactions can, therefore, occur within adjacent wells without intermixing reaction fluids and chemically distinct test solutions can be applied to individual wells.

This technology is useful in applications where large numbers of biomolecular interactions are measured in parallel, particularly when molecular labels would alter or inhibit the functionality of the molecules under study. High-throughput screening of pharmaceutical compound libraries with protein targets, and microarray screening of protein-protein interactions for proteomics are examples of applications that require the sensitivity and throughput afforded by the compositions and methods of the invention.

Unlike surface plasmon resonance, resonant mirrors, and waveguide biosensors, the described compositions and methods enable many thousands of individual binding reactions to take place simultaneously upon the biosensor surface. This technology is useful in applications where large numbers of biomolecular interactions are measured in parallel (such as in an array), particularly when molecular labels alter or inhibit the functionality of the molecules under study. These biosensors are especially suited for high-throughput screening of pharmaceutical compound libraries with protein targets, and microarray screening of protein-protein interactions for proteomics. A biosensor of the invention can be manufactured, for example, in large areas using a plastic embossing process, and thus can be inexpensively incorporated into common disposable laboratory assay platforms such as microtiter plates and microarray slides.

Other similar biosensors may also be used in the instant invention. Numerous biosensors have been developed to detect a variety of biomolecular complexes including oligonucleotides, antibody-antigen interactions, hormone-receptor interactions, and enzyme-substrate interactions. In general, these biosensors consist of two components: a highly specific recognition element and a transducer that converts the molecular recognition event into a quantifiable signal. Signal transduction has been accomplished by many methods, including fluorescence, interferometry (Jenison et al., “Interference-based detection of nucleic acid targets on optically coated silicon,” Nature Biotechnology, 19, p. 62-65; Lin et al., “A porous silicon-based optical interferometric biosensor,” Science, 278, p. 840-843, 1997), and gravimetry (A. Cunningham, Bioanalytical Sensors, John Wiley & Sons (1998)). Of the optically-based transduction methods, direct methods that do not require labeling of analytes with fluorescent compounds are of interest due to the relative assay simplicity and ability to study the interaction of small molecules and proteins that are not readily labeled.

These direct optical methods include surface plasmon resonance (SPR) (Jordan & Corn, “Surface Plasmon Resonance Imaging Measurements of Electrostatic Biopolymer Adsorption onto Chemically Modified Gold Surfaces,” Anal. Chem., 69:1449-1456 (1997); plasmom-resonant particles (PRPs) (Schultz et al., Proc. Nat. Acad. Sci., 97: 996-1001 (2000); grating couplers (Morhard et al., “Immobilization of antibodies in micropatterns for cell detection by optical diffraction,” Sensors and Actuators B, 70, p. 232-242, 2000); ellipsometry (Jin et al., “A biosensor concept based on imaging ellipsometry for visualization of biomolecular interactions,” Analytical Biochemistry, 232, p. 69-72, 1995), evanascent wave devices (Huber et al., “Direct optical immunosensing (sensitivity and selectivity),” Sensors and Actuators B, 6, p. 122.126, 1992), resonance light scattering (Bao et al., Anal. Chem., 74:1792-1797 (2002), and reflectometry (Brecht & Gauglitz, “Optical probes and transducers,” Biosensors and Bioelectronics, 10, p. 923-936, 1995). Changes in the optical phenomenon of surface plasmon resonance (SPR) can be used as an indication of real-time reactions between biological molecules. Theoretically predicted detection limits of these detection methods have been determined and experimentally confirmed to be feasible down to diagnostically relevant concentration ranges.

Surface plasmon resonance (SPR) has been successfully incorporated into an immunosensor format for the simple, rapid, and nonlabeled assay of various biochemical analytes. Proteins, complex conjugates, toxins, allergens, drugs, and pesticides can be determined directly using either natural antibodies or synthetic receptors with high sensitivity and selectivity as the sensing element. Immunosensors are capable of real-time monitoring of the antigen-antibody reaction. A wide range of molecules can be detected with lower limits ranging between 10⁻⁹ and 10⁻¹³ mol/L. Several successful commercial developments of SPR immunosensors are available and their web pages are rich in technical information. Wayne et al. (Methods 22: 77-91, 2000) reviewed and highlighted many recent developments in SPR-based immunoassay, functionalizations of the gold surface, novel receptors in molecular recognition, and advanced techniques for sensitivity enhancement.

Utilization of the optical phenomenon surface plasmon resonance (SPR) has seen extensive growth since its initial observation by Wood in 1902 (Phil. Mag. 4 (1902), pp. 396-402). SPR is a simple and direct sensing technique that can be used to probe refractive index (η) changes that occur in the very close vicinity of a thin metal film surface (Otto Z. Phys. 216 (1968), p. 398). The sensing mechanism exploits the properties of an evanescent field generated at the site of total internal reflection. This field penetrates into the metal film, with exponentially decreasing amplitude from the glass-metal interface. Surface plasmons, which oscillate and propagate along the upper surface of the metal film, absorb some of the plane-polarized light energy from this evanescent field to change the total internal reflection light intensity Iᵣ. A plot of Iᵣ versus incidence (or reflection) angle θ produces an angular intensity profile that exhibits a sharp dip. The exact location of the dip minimum (or the SPR angle θᵣ) can be determined by using a polynomial algorithm to fit the Iᵣ signals from a few diodes close to the minimum. The binding of molecules on the upper metal surface causes a change in η of the surface medium that can be observed as a shift in θᵣ.

The potential of SPR for biosensor purposes was realized in 1982-1983 by Liedberg et al., who adsorbed an immunoglobulin G (IgG) antibody overlayer on the gold sensing film, resulting in the subsequent selective binding and detection of IgG (Nylander et al., Sens. Actuators 3 (1982), pp. 79-84; Liedberg et al., Sens. Actuators 4 (1983), pp. 229-304). The principles of SPR as a biosensing technique have been reviewed previously (Daniels et al., Sens. Actuators 15 (1988), pp. 11-18; VanderNoot and Lai, Spectroscopy 6 (1991), pp. 28-33; Lundström Biosens. Bioelectron. 9 (1994), pp. 725-736; Liedberg et al., Biosens. Bioelectron. 10 (1995); Morgan et al., Clin. Chem. 42 (1996), pp. 193-209; Tapuchi et al., S. Afr. J. Chem. 49 (1996), pp. 8-25). Applications of SPR to biosensing were demonstrated for a wide range of molecules, from virus particles to sex hormone-binding globulin and syphilis. Most importantly, SPR has an inherent advantage over other types of biosensors in its versatility and capability of monitoring binding interactions without the need for fluorescence or radioisotope labeling of the biomolecules. This approach has also shown promise in the real-time determination of concentration, kinetic constant, and binding specificity of individual biomolecular interaction steps. Antibody-antigen interactions, peptide/protein-protein interactions, DNA hybridization conditions, biocompatibility studies of polymers, biomolecule-cell receptor interactions, and DNA/receptor-ligand interactions can all be analyzed (Pathak and Savelkoul, Immunol. Today 18 (1997), pp. 464-467). Commercially, the use of SPR-based immunoassay has been promoted by companies such as Biacore (Uppsala, Sweden) (Jonsson et al., Ann. Biol. Clin. 51 (1993), pp. 19-26), Windsor Scientific (U.K.) (WWW URL for Windsor Scientific IBIS Biosensor), Quantech (Minnesota) (WWW URL for Quantech), and Texas Instruments (Dallas, Tex.) (WWW URL for Texas Instruments).

In another related embodiment, the binding event between the capture agents and the analyte can be detected by using a water-soluble luminescent quantum dot as described in U.S. 2003/0008414A1 (incorporated herein by reference). In one embodiment, a water-soluble luminescent semiconductor quantum dot comprises a core, a cap and a hydrophilic attachment group. The “core” is a nanoparticle-sized semiconductor. While any core of the IIB-VIB, IIIB-VB or IVB-IVB semiconductors can be used in this context, the core must be such that, upon combination with a cap, a luminescent quantum dot results. A IIB-VIB semiconductor is a compound that contains at least one element from Group IEB and at least one element from Group VIB of the periodic table, and so on. Preferably, the core is a IIB-VIB, IIIB-VB or IVB-IVB semiconductor that ranges in size from about 1 nm to about 10 nm. The core is more preferably a IIB-VIB semiconductor and ranges in size from about 2 mm to about 5 nm. Most preferably, the core is CdS or CdSe. In this regard, CdSe is especially preferred as the core, in particular at a size of about 4.2 nm.

The “cap” is a semiconductor that differs from the semiconductor of the core and binds to the core, thereby forming a surface layer on the core. The cap must be such that, upon combination with a given semiconductor core, results in a luminescent quantum dot. The cap should passivate the core by having a higher band gap than the core. In this regard, the cap is preferably a IIB-VIB semiconductor of high band gap. More preferably, the cap is ZnS or CdS. Most preferably, the cap is ZnS. In particular, the cap is preferably ZnS when the core is CdSe or CdS and the cap is preferably CdS when the core is CdSe.

The “attachment group” as that term is used herein refers to any organic group that can be attached, such as by any stable physical or chemical association, to the surface of the cap of the luminescent semiconductor quantum dot and can render the quantum dot water-soluble without rendering the quantum dot no longer luminescent. Accordingly, the attachment group comprises a hydrophilic moiety. Preferably, the attachment group enables the hydrophilic quantum dot to remain in solution for at least about one hour, one day, one week, or one month. Desirably, the attachment group is attached to the cap by covalent bonding and is attached to the cap in such a manner that the hydrophilic moiety is exposed. Preferably, the hydrophilic attachment group is attached to the quantum dot via a sulfur atom. More preferably, the hydrophilic attachment group is an organic group comprising a sulfur atom and at least one hydrophilic attachment group. Suitable hydrophilic attachment groups include, for example, a carboxylic acid or salt thereof, a sulfonic acid or salt thereof, a sulfamic acid or salt thereof, an amino substituent, a quaternary ammonium salt, and a hydroxy. The organic group of the hydrophilic attachment group of the present invention is preferably a C1-C6 alkyl group or an aryl group, more preferably a C1-C6 alkyl group, even more preferably a C1-C3 alkyl group. Therefore, in a preferred embodiment, the attachment group of the present invention is a thiol carboxylic acid or thiol alcohol. More preferably, the attachment group is a thiol carboxylic acid. Most preferably, the attachment group is mercaptoacetic acid.

Accordingly, a preferred embodiment of a water-soluble luminescent semiconductor quantum dot is one that comprises a CdSe core of about 4.2 nm in size, a ZnS cap and an attachment group. Another preferred embodiment of a water soluble luminescent semiconductor quantum dot is one that comprises a CdSe core, a ZnS cap and the attachment group mercaptoacetic acid. An especially preferred water-soluble luminescent semiconductor quantum dot comprises a CdSe core of about 4.2 nm, a ZnS cap of about 1 nm and a mercaptoacetic acid attachment group.

The capture agent of the instant invention can be attached to the quantum dot via the hydrophilic attachment group and forms a conjugate. The capture agent can be attached, such as by any stable physical or chemical association, to the hydrophilic attachment group of the water-soluble luminescent quantum dot directly or indirectly by any suitable means, through one or more covalent bonds, via an optional linker that does not impair the function of the capture agent or the quantum dot. For example, if the attachment group is mercaptoacetic acid and a nucleic acid biomolecule is being attached to the attachment group, the linker preferably is a primary amine, a thiol, streptavidin, neutravidin, biotin, or a like molecule. If the attachment group is mercaptoacetic acid and a protein biomolecule or a fragment thereof is being attached to the attachment group, the linker preferably is streptavidin, neutravidin, biotin, or a like molecule.

By using the quantum dot-capture agent conjugate, an immobilized analyte, when in contact with a conjugate as described above, will promote the emission of luminescence when the capture agent of the conjugate specifically binds to the analyte. This is particularly useful when the capture agent is a nucleic acid aptamer or an antibody. When the aptamer is used, an alternative embodiment may be employed, in which a fluorescent quencher may be positioned adjacent to the quantum dot via a self-pairing stem-loop structure when the aptamer is not bound to an analyte. When the aptamer binds to the analyte, the stem-loop structure is opened, thus releasing the quenching effect and generates luminescence.

In another related embodiment, arrays of nanosensors comprising nanowires or nanotubes as described in U.S. 2002/0117659A1 may be used for detection and/or quantitation of analyte-capture agent interaction. Briefly, a “nanowire” is an elongated nanoscale semiconductor, which can have a cross-sectional dimension of as thin as 1 nanometer. Similarly, a “nanotube” is a nanowire that has a hollowed-out core, and includes those nanotubes know to those of ordinary skill in the art. A “wire” refers to any material having a conductivity at least that of a semiconductor or metal. These nanowires/nanotubes may be used in a system constructed and arranged to determine an analyte (e.g., capture agent) in a sample to which the nanowire(s) is exposed. The surface of the nanowire is functionalized by coating with an analyte. Binding of an analyte to the functionalized nanowire causes a detectable change in electrical conductivity of the nanowire or optical properties. Thus, presence of the analyte can be determined by determining a change in a characteristic in the nanowire, typically an electrical characteristic or an optical characteristic. A variety of biomolecular entities can be used for coating, including, but not limited to, amino acids, proteins, sugars, DNA, antibodies, antigens, and enzymes, etc. For more details such as construction of nanowires, functionalization with various biomolecules (such as the capture agents of the instant invention), and detection in nanowire devices, see U.S. 2002/0117659A1 (incorporated by reference). Since multiple nanowires can be used in parallel, each with a different analyte as the functionalized group, this technology is ideally suited for large scale arrayed detection of analytes in biological samples without the need to label the analytes. This nanowire detection technology has been successfully used to detect pH change (H+ binding), biotin-streptavidin binding, antibody-antigen binding, metal (Ca 2+) binding with picomolar sensitivity and in real time (Cui et al., Science 293: 1289-1292).

Matrix-assisted laser desorption/ionization time-of-flight mass spectrometry (MALDI-TOF MS), uses a laser pulse to desorb proteins from the surface followed by mass spectrometry to identify the molecular weights of the proteins (Gilligan et al., Mass spectrometry after capture and small-volume elution of analyte from a surface plasmon resonance biosensor. Anal. Chem. 74 (2002), pp. 2041-2047). Because this method only measures the mass of proteins at the interface, and because the desorption protocol is sufficiently mild that it does not result in fragmentation, MALDI can provide straightforward useful information such as confirming the identity of the bound capture agents. For this matter, MALDI can be used to identify proteins that are bound to immobilized analytess.

VI. Miscellaneous

Samples and Their Preparation

If the target analytes include proteins (not just small molecules/metabolites), the sample containing these target analytes is preferably pre-treated for use with the PET-peptide containing arrays. The protein targets to be analyzed in a sample, e.g., a biological fluid, a water sample, or a food sample, are typically fragmented to generate a collection of peptides, under conditions suitable for binding a PET corresponding to a protein of interest.

Even if all interested analytes are non-peptide small molecules/metabolites, treatment of the sample may be advantageous since the treatment simplifies the complexity of the sample, eliminating such potential interfering factors as anti-animal antibodies, and/or natural proteins bound to and/or acts on interested metabolites (enzymes, etc.).

The co-pending U.S. Ser. No. 60/519,530 describes in detail about various sample preparation methods, the content of which are incorporated herein by reference.

For all embodiments, samples to be used for the assay of the present invention may be drawn from various physiological, environmental or artificial sources. In particular, physiological samples such as body fluids or tissue samples of a patient or an organism may be used as assay samples. Such fluids include, but are not limited to, saliva, mucous, sweat, whole blood, serum, urine, amniotic fluid, genital fluids, fecal material, marrow, plasma, spinal fluid, pericardial fluids, gastric fluids, abdominal fluids, peritoneal fluids, pleural fluids and extraction from other body parts, and secretion from other glands. Alternatively, biological samples drawn from cells taken from the patient or grown in culture may be employed. Such samples include supernatants, whole cell lysates, or cell fractions obtained by lysis and fractionation of cellular material. Extracts of cells and fractions thereof, including those directly from a biological entity and those grown in an artificial environment, can also be used. In addition, a biological sample can be obtained and/or deribed from, for example, blood, plasma, serum, gastrointestinal secretions, homogenates of tissues or tumors, synovial fluid, feces, saliva, sputum, cyst fluid, amniotic fluid, cerebrospinal fluid, peritoneal fluid, lung lavage fluid, semen, lymphatic fluid, tears, or prostatitc fluid.

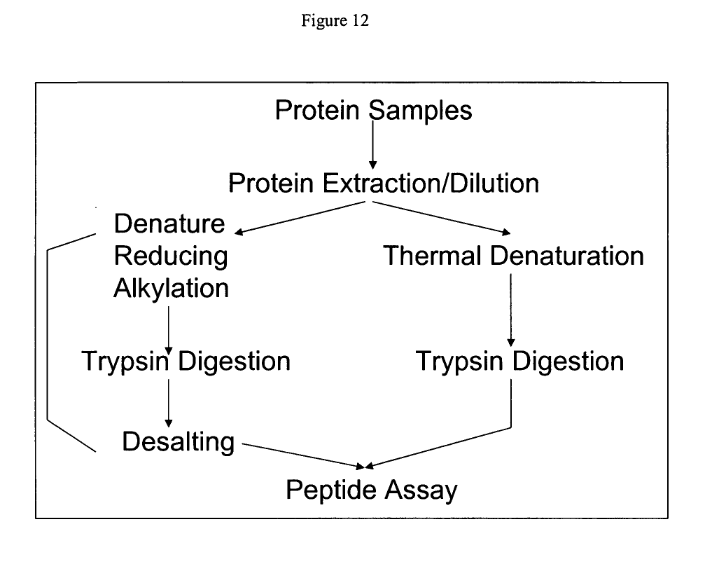

A general scheme of sample preparation prior to its use in the methods of the instant invention is described in FIG. 12. Briefly, a sample can be pretreated by extraction and/or dilution to minimize the interference from certain substances present in the sample. The sample can then be either chemically reduced, denatured, alkylated, or subjected to thermo-denaturation. Regardless of the denaturation step, the denatured sample is then digested by a protease, such as trypsin, before it is used in subsequent assays. A desalting step may also be added just after protease digestion if chemical denaturation if used. This process is generally simple, robust and reproducible, and is generally applicable to main sample types including serum, cell lysates and tissues.

The sample may be pre treated to remove extraneous materials, stabilized, buffered, preserved, filtered, or otherwise conditioned as desired or necessary. Proteins in the sample typically are fragmented, either as part of the methods of the invention or in advance of performing these methods. Fragmentation can be performed using any art-recognized desired method, such as by using chemical cleavage (e.g., cyanogen bromide); enzymatic means (e.g., using a protease such as trypsin, chymotrypsin, pepsin, papain, carboxypeptidase, calpain, subtilisin, gluc-C, endo lys-C and proteinase K, or a collection or sub-collection thereof); or physical means (e.g., fragmentation by physical shearing or fragmentation by sonication). As used herein, the terms “fragmentation” “cleavage,” “proteolytic cleavage,” “proteolysis” “restriction” and the like are used interchangeably and refer to scission of a chemical bond, typically a peptide bond, within proteins to produce a collection of peptides (i.e., protein fragments).

The purpose of the fragmentation is to generate competition peptides comprising PET which are soluble and available for binding with a capture agent. In essence, the sample preparation is designed to assure to the extent possible that all PET present on or within relevant proteins that may be present in the sample are available for competition binding to the capture agents with the immobilized PET-containing peptides. This strategy can avoid many of the problems encountered with previous attempts to design protein chips caused by protein-protein complexation, post translational modifications and the like.

In one embodiment, the sample of interest is treated using a pre-determined protocol which: (A) inhibits masking of the target protein caused by target protein-protein non covalent or covalent complexation or aggregation, target protein degradation or denaturing, target protein post-translational modification, or environmentally induced alteration in target protein tertiary structure, and (B) fragments the target protein to, thereby, produce at least one peptide epitope (i.e., a PET) whose concentration is directly proportional to the true concentration of the target protein in the sample. The sample treatment protocol is designed and empirically tested to result reproducibly in the generation of a PET that is available for competitive binding with a given capture agent. The treatment can involve protein separations; protein fractionations; solvent modifications such as polarity changes, osmolarity changes, dilutions, or pH changes; heating; freezing; precipitating; extractions; reactions with a reagent such as an endo-, exo- or site specific protease; non proteolytic digestion; oxidations; reductions; neutralization of some biological activity, and other steps known to one of skill in the art.

For example, the sample may be treated with an alkylating agent and a reducing agent in order to prevent the formation of dimers or other aggregates through disulfide/dithiol exchange. The sample of PET-containing peptides may also be treated to remove secondary modifications, including but are not limited to, phosphorylation, methylation, glycosylation, acetylation, prenylation, using, for example, respective modification-specific enzymes such as phosphatases, etc.

In one embodiment, proteins of a sample will be denatured, reduced and/or alkylated, but will not be proteolytically cleaved. Proteins can be denatured by thermal denaturation or organic solvents, then subjected to direct detection or optionally, further proteolytic cleavage.

The use of thermal denaturation (50-90° C. for about 20 minutes) of proteins prior to enzyme digestion in solution is preferred over chemical denaturation (such as 6-8 M guanidine HCl or urea) because it does not require purification/concentration, which might be preferred or required prior to subsequent analysis. Park and Russell reported that enzymatic digestions of proteins that are resistant to proteolysis are significantly enhanced by thermal denaturation (Anal. Chem., 72 (11): 2667-2670, 2000). Native proteins that are sensitive to proteolysis show similar or just slightly lower digestion yields following thermal denaturation. Proteins that are resistant to digestion become more susceptible to digestion, independent of protein size, following thermal denaturation. For example, amino acid sequence coverage from digest fragments increases from 15 to 86% in myoglobin and from 0 to 43% in ovalbumin. This leads to more rapid and reliable protein identification by the instant invention, especially to protease resistant proteins.

Although some proteins aggregate upon thermal denaturation, the protein aggregates are easily digested by trypsin and generate sufficient numbers of digest fragments for protein identification. In fact, protein aggregation may be the reason thermal denaturation facilitates digestion in most cases. Protein aggregates are believed to be the oligomerization products of the denatured form of protein (Copeland, R. A. Methods for Protein Analysis; Chapman & Hall: New York, N.Y., 1994). In general, hydrophobic parts of the protein are located inside and relatively less hydrophobic parts of the protein are exposed to the aqueous environment. During the thermal denaturation, intact proteins are gradually unfolded into a denatured conformation and sufficient energy is provided to prevent a fold back to its native conformation. The probability for interactions with other denatured proteins is increased, thus allowing hydrophobic interactions between exposed hydrophobic parts of the proteins. In addition, protein aggregates of the denatured protein can have a more protease-labile structure than nondenatured proteins because more cleavage sites are exposed to the environment. Protein aggregates are easily digested, so that protein aggregates are not observed at the end of 3 h of trypsin digestion (Park and Russell, Anal. Chem., 72 (11): 2667-2670, 2000). Moreover, trypsin digestion of protein aggregates generates more specific cleavage products.

Ordinary proteases such as trypsin may be used after denaturation. The process may be repeated by one or more rounds after the first round of denaturation and digestion. Alternatively, this thermal denaturation process can be further assisted by using thermophilic trypsin-like enzymes, so that denaturation and digestion can be done simultaneously. For example, Nongporn Towatana et al. (J of Bioscience and Bioengineering 87(5): 581-587, 1999) reported the purification to apparent homogeneity of an alkaline protease from culture supernatants of Bacillus sp. PS719, a novel alkaliphilic, thermophilic bacterium isolated from a thermal spring soil sample. The protease exhibited maximum activity towards azocasein at pH 9.0 and at 75° C. The enzyme was stable in the pH range 8.0 to 10.0 and up to 80° C. in the absence of Ca²⁺. This enzyme appears to be a trypsin-like serine protease, since phenylmethylsulfonyl fluoride (PMSF) and 3,4-dichloroisocoumarin (DCI) in addition to N-α-p-tosyl-L-lysine chloromethyl ketone (TLCK) completely inhibited the activity. Among the various oligopeptidyl-p-nitroanilides tested, the protease showed a preference for cleavage at arginine residues on the carboxylic side of the scissile bond of the substrate, liberating p-nitroaniline from N-carbobenzoxy (CBZ)-L-arginine-p-nitroanilide with the Kₘ and Vₘₐₓ values of 0.6 mM and 1.0 μmol min⁻¹ mg protein⁻¹, respectively.

Alternatively, existing proteases may be chemically modified to achieve enhanced thermostability for use in this type of application. Mozhaev et al. (Eur J Biochem. 173(1):147-54, 1988) experimentally verified the idea presented earlier that the contact of nonpolar clusters located on the surface of protein molecules with water destabilizes proteins. It was demonstrated that protein stabilization could be achieved by artificial hydrophilization of the surface area of protein globules by chemical modification. Two experimental systems were studied for the verification of the hydrophilization approach. In one experiment, the surface tyrosine residues of trypsin were transformed to aminotyrosines using a two-step modification procedure: nitration by tetranitromethane followed by reduction with sodium dithionite. The modified enzyme was much more stable against irreversible thermo-inactivation: the stabilizing effect increased with the number of aminotyrosine residues in trypsin and the modified enzyme could become even 100 times more stable than the native one. In another experiment, alpha-chymotrypsin was covalently modified by treatment with anhydrides or chloroanhydrides of aromatic carboxylic acids. As a result, different numbers of additional carboxylic groups (up to five depending on the structure of the modifying reagent) were introduced into each Lys residue modified. Acylation of all available amino groups of alpha-chymotrypsin by cyclic anhydrides of pyromellitic and mellitic acids resulted in a substantial hydrophilization of the protein as estimated by partitioning in an aqueous Ficoll-400/Dextran-70 biphasic system. These modified enzyme preparations were extremely stable against irreversible thermal inactivation at elevated temperatures (65-98° C.); their thermostability was practically equal to the stability of proteolytic enzymes from extremely thermophilic bacteria, the most stable proteinases known to date. Similar approaches may be used to any other chosen proteases for the subject method.

In certain embodiments, immobilized enzymes may be used as a means to: a) speed up the digestion, and b) decrease the presence of fragments of trypsin or other proteases in the sample that goes on to further analysis steps.

In other embodiments, samples can be pre-treated with reducing agents such as mercaptoethanol or DTT to reduce the disulfide bonds to facilitate digestion.

Fractionation may be performed using any single or multidimentional chromatography, such as reversed phase chromatography (RPC), ion exchange chromatography, hydrophobic interaction chromatography, size exclusion chromatography, or affinity fractionation such as immunoaffinity and immobilized metal affinity chromatography. Preferably, the fractionation involves surface-mediated selection strategies. Electrophoresis, either slab gel or capillary electrophoresis, can also be used to fractionate the peptides in the sample. Examples of slab gel electrophoretic methods include sodium dodecyl sulfate polyacrylamide gel electrophoresis (SDS-PAGE) and native gel electrophoresis. Capillary electrophoresis methods that can be used for fractionation include capillary gel electrophoresis (CGE), capillary zone electrophoresis (CZE) and capillary electrochromatography (CEC), capillary isoelectric focusing, immobilized metal affinity chromatography and affinity electrophoresis.

Protein precipitation may be performed using techniques well known in the art. For example, precipitation may be achieved using known precipitants, such as potassium thiocyanate, trichloroacetic acid and ammonium sulphate.

Subsequent to fragmentation, the sample may be contacted with the capture agents and the immobilized peptide arrays of the present invention, e.g., PET-containing peptide arrays immobilized on a planar support or on a bead, as described herein. Alternatively, the fragmented sample (containing a collection of peptides) may be fractionated based on, for example, size, post-translational modifications (e.g., glycosylation or phosphorylation) or antigenic properties, and then contacted with the capture agents and the immobilized peptide arrays of the present invention, e.g., PET-containing peptide arrays immobilized on a planar support or on a bead.

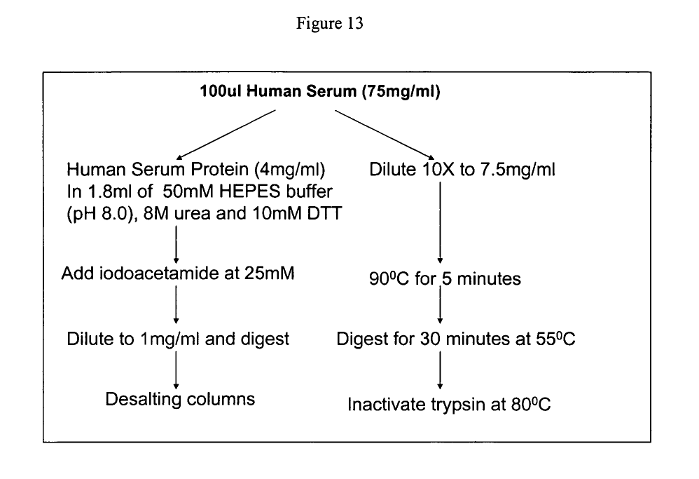

FIG. 13 provides an illustrative example of serum sample pre-treatment using either the thermo-denaturation or the chemical denaturation. Briefly, for thermo-denaturation, 100 μL of human serum (about 75 mg/mL total protein) is first diluted 10-fold to about 7.5 mg/mL. The diluted sample is then heated to 90° C. for 5 minutes to denature the proteins, followed by 30 minutes of trypsin digestion at 55° C. The trypsin is inactivated at 80° C. after the digestion.

For chemical denaturation, about 1.8 mL of human serum proteins diluted to about 4 mg/mL is denatured in a final concentration of 50 mM HEPES buffer (pH 8.0), 8M urea and 10 mM DTT. Iodoacetamide is then added to 25 mM final concentration. The denatured sample is then further diluted to about 1 mg/mL for protease digestion. The digested sample will pass through a desalting column before being used in subsequent assays.

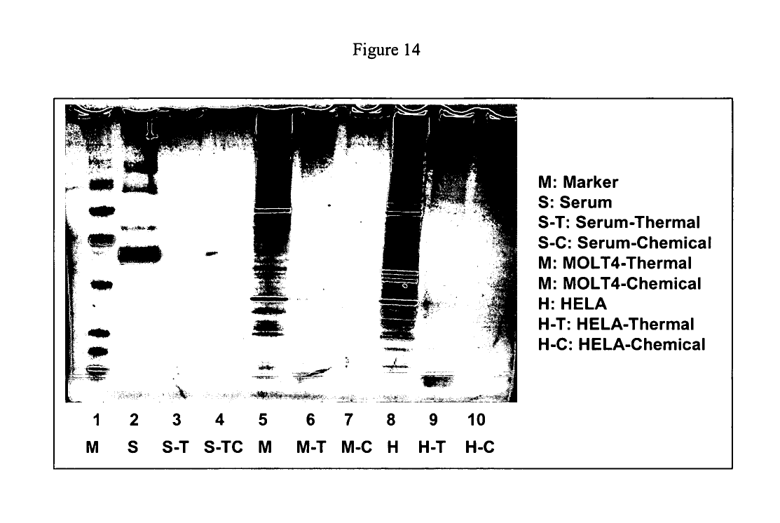

FIG. 14 shows the result of thermo-denaturation and chemical denaturation of serum proteins, cell lysates (MOLT4 and Hela cells). It is evident that denaturation was successful for the majority, if not all of the proteins in both the thermo- and chemical-denaturation lanes, and both methods achieved comparable results in terms of protein denaturation and fragmentation.

The above example is for illustrative purpose only and is by no means limiting. Minor alterations of the protocol depending on specific uses can be easily achieved for optimal results in individual assays.

Selection of PET

One advantages of the PET of the instant invention is that PET can be determined in sillico and generated in vitro (such as by peptide synthesis) without cloning or purifying the protein it belongs. PET is also advantageous over the full-length tryptic fragments (or for that matter, any other fragments that predictably results from any other treatments) since full-length tryptic fragments tend to contain one or more PETs themselves, though the tryptic fragment itself may be unique simply because of its length (the longer a stretch of peptide, the more likely it will be unique). A direct implication is that, by using relatively short and unique PETs rather than the full-length (tryptic) peptide fragments, the method of the instant invention has greatly reduced, if not completely eliminated, the risk of having multiple antibodies with unique specificities against the same peptide fragment—a source of antibody cross-reactivity. An additional advantage may be added due to the PET selection process, such as the nearest-neighbor analysis and ranking prioritization(see below), which further eliminates the chance of cross-reactivity. All these features make the PET-based methods particularly suitable for genome-wide analysis using multiplexing techniques.

The PET of the instant invention can be selected in various ways. In the simplest embodiment, the PET for a given organism or biological sample can be generated or identified by a brute force search of the relevant database, using all theoretically possible PET with a given length. This process is preferably carried out computationaly using, for example, any of the sequence search tools available in the art or variations thereof. For example, to identify PET of 5 amino acids in length (a total of 3.2 million possible PET candidates, see table 2.2.2 below), each of the 3.2 million candidates may be used as a query sequence to search against the human proteom as described below. Any candidate that has more than one hit (found in two or more proteins) is immediately eliminated before further searching is done. At the end of the search, a list of human proteins that have one or more PETs can be obtained (see Example 1 below). The same or similar procedure can be used for any pre-determined organism or database.

For example, PETs for each human protein can be identified using the following procedure. A Perl program is developed to calculate the occurrence of all possible peptides, given by 20^{N}, of defined length N (amino acids) in human proteins. For example, the total tag space is 160,000 (20⁴) for tetramer peptides, 3.2 M (20⁵) for pentamer peptides, and 64 M (20⁶) for hexamer peptides, so on. Predicted human protein sequences are analyzed for the presence or absence of all possible peptides of N amino acids. PET are the peptide sequences that occur only once in the human proteome. Thus the presence of a specific PET is an intrinsic property of the protein sequence and is operational independent. According to this approach, a definitive set of PETs can be defined and used regardless of the sample processing procedure (operational independence).

In one embodiment, to speed up the searching process, computer algorithms may be developed or modified to eliminate unnecessary searches before the actual search begins.

Using the example above, two highly related (say differ only in a few amino acid positions) human proteins may be aligned, and a large number of candidate PET can be eliminated based on the sequence of the identical regions. For example, if there is a stretch of identical sequence of 20 amino acids, then sixteen 5-amino acid PETs can be eliminated without searching, by virtue of their simultaneous appearance in two non-identical human proteins. This elimination process can be continued using as many highly related protein pairs or families as possible, such as the evolutionary conserved proteins such as histones, globins, etc.

In another embodiment, the identified PET for a given protein may be rank-ordered based on certain criteria, so that higher ranking PETs are preferred to be used in generating specific capture agents.

For example, certain PET may naturally exist on protein surface, thus making good candidates for being a soluble peptide when digested by a protease. On the other hand, certain PET may exist in an internal or core region of a protein, and may not be readily soluble even after digestion. Such solubility property may be evaluated by available softwares. The solvent accessibility method described in Boger, J., Emini, E. A. & Schmidt, A., Surface probability profile—An heuristic approach to the selection of synthetic peptide antigens, Reports on the Sixth International Congress in Immunology (Toronto) 1986 p. 250 also may be used to identify PETs that are located on the surface of the protein of interest. The package MOLMOL (Koradi, R. et al. (1996) J. Mol. Graph. 14:51-55) and Eisenhaber's ASC method (Eisenhaber and Argos (1993) J. Comput. Chem. 14:1272-1280; Eisenhaber et al. (1995) J. Comput. Chem. 16:273-284) may also be used. Surface PETs generally have higher ranking than internal PETS. In one embodiment, the logP or logD values that can be calculated for a PET, or proteolytic fragment containing a PET, can be calculated and used to rank order the PET's based on likely solubility under conditions that a protein sample is to be contacted with a capture agent.

Regardless of the manner the PETs are generated, an ideal PET preferably is 8 amino acids in length, and the parental tryptic peptide should be smaller than 20 amino acid long. This is because antibodies typically recognize peptide epitopes of 4-8 amino acids, thus peptides of 12-20 amino acids are conventionally used for antibody production.

Since trypsin is a preferred digestion enzyme in certain embodiments, a PET in these embodiments should not contain K or R in the middle of the sequence so that the PET will not be cleaved by trypsin during sample preparation. In a more general sense, the selected PET should not contain or overlap a digestion site such that the PET is expected to be destroyed after digestion, unless an assay specifically prefer that a PET be destroyed after digestion.

In addition, an ideal PET preferably does not have hydrophobic parental tryptic peptide, is highly antigenic, and has the smallest numbers (preferably none) of closest related peptides (nearest neighbor peptides or NNP) defined by nearest neighbor analysis.

Any PET may also be associated with an annotation, which may contain useful information such as: whether the PET may be destroyed by a certain protease (such as trypsin), whether it is likely to appear on a digested peptide with a relatively rigid or flexible structure, etc. These characteristics may help to rank order the PETs for use if generating specific capture agents, especially when there are a large number of PETs associated with a given protein. Since PET may change depending on particular use in a given organism, ranking order may change depending on specific usages. A PET may be low ranking due to its probability of being destroyed by a certain protease may rank higher in a different fragmentation scheme using a different protease.

In another embodiment, the computational algorithm for selecting optimal PET from a protein for antibody generation takes antibody-peptide interaction data into consideration. A process such as Nearest-Neighbor Analysis (NNA), can be used to select most unique PET for each protein. Each PET in a protein is given a relative score, or PET Uniqueness Index, that is based on the number of nearest neighbors it has. The higher the PET Uniqueness Index, the more unique the PET is. The PET Uniqueness Index can be calculated using an Amino Acid Replacement Matrix such as the one in Table VIII of Getzoff, E D, Tainer J A and Lerner R A. The chemistry and meachnism of antibody binding to protein antigens. 1988. Advances. Immunol. 43: 1-97. In this matrix, the replaceability of each amino acid by the remaining 19 amino acids was calculated based on experimental data on antibody cross-reactivity to a large number of peptides of single mutations (replacing each amino acid in a peptide sequence by the remaining 19 amino acids). For example, each octamer PET from a protein is compared to 8.7 million octamers present in human proteome and a PET Uniqueness Index is calculated. This process not only selects the most unique PET for particular protein, it also identifies Nearest Neighbor Peptides for this PET. This becomes important for defining cross-reactivity of PET-specific antibodies since Nearest Neighbor Peptides are the ones most likely will cross-react with particular antibody.

Besides PET Uniqueness Index, the following parameters for each PET may also be calculated and help to rank the PETs:
a) PET Solubility Index: which involves calculating LogP and LogD of the PET.
b) PET Hydrophobicity & water accessibility: only hydrophilic peptides and peptides with good water accessibility will be selected.
c) PET Length: since longer peptides tend to have conformations in solution, we use PET peptides with defined length of 8 amino acids. PET-specific antibodies will have better defined specificity due to limited number of epitopes in a shorter peptide sequences. This is very important for multiplexing assays using these antibodies. In one embodiment, only antibodies generated by this way will be used for multiplexing assays.
d) Evolutionary Conservation Index: each human PET will be compared with other species to see whether a PET sequence is conserved cross species. Ideally, PET with minimal conservation, for example, between mouse and human sequences will be selected. This will maximize the possibility to generate good immunoresponse and monoclonal antibodies in mouse.
   VII. Applications of the Invention

A. Investigative and Diagnostic Applications

The microarrays of the present invention provides a powerful tool in probing living systems and in diagnostic applications (e.g., clinical, environmental and industrial, and food safety diagnostic applications). For clinical diagnostic applications, the arrays may be used to detect the concentration or changes thereof in one or more diagnostic targets in a biological sample (e.g., a disease related protein or small molecule metabolites, collection or pattern of proteins and/or metabolites). Specific individual disease related proteins include, for example, prostate-specific antigen (PSA), prostatic acid phosphatase (PAP) or prostate specific membrane antigen (PSMA) (for diagnosing prostate cancer); Cyclin E for diagnosing breast cancer; Annexin, e.g., Annexin V (for diagnosing cell death in, for example, cancer, ischemia, or transplant rejection); or β-amyloid plaques (for diagnosing Alzheimer's Disease).

For example, the subject arrays can be used to identify potential biomarkers as surrogate end points in developing new drugs, monitoring treatment efficacy or disease progression, and prediction of clinical outcomes. There is a high level of interest in biomarkers in the pharmaceutical industry, which is faced with the ever increasing cost of research and development, and with growing pressure to accelerate the rate of bringing new drugs to the marketplace. In this context, biomarkers show considerable promise for improving the efficiency and informativeness of drug development and regulatory decision making.

“Biological marker (biomarker)” refers to a physical sign or laboratory measurement that occurs in association with a pathological process and that has putative diagnostic and/or prognostic utility.

“Surrogate endpoint” (or “surrogate marker”) is a biomarker that is intended to serve as a substitute for a clinically meaningful endpoint and is expected to predict the effect of a therapeutic intervention. It is an objective biochemical marker which correlates with the absence or presence of a disease or disorder, or with the progression of a disease or disorder (e.g., with the presence or absence of a tumor). The presence or quantity of such markers is independent of the causation of the disease. Therefore, these markers may serve to indicate whether a particular course of treatment is effective in lessening a disease state or disorder. Surrogate markers are of particular use when the presence or extent of a disease state or disorder is difficult to assess through standard methodologies (e.g., early stage tumors), or when an assessment of disease progression is desired before a potentially dangerous clinical endpoint is reached (e.g., an assessment of cardiovascular disease may be made using an analyte corresponding to a protein associated with a cardiovascular disease as a surrogate marker, and an analysis of HIV infection may be made using an analyte corresponding to an HIV protein as a surrogate marker, well in advance of the undesirable clinical outcomes of myocardial infarction or fully-developed AIDS). Examples of the use of surrogate markers in the art include: Koomen et al. (2000) J. Mass. Spectrom. 35:258-264; and James (1994) AIDS Treatment News Archive 209.

“Clinical endpoint” is a clinically meaningful measure of how a patient feels, functions, or survives.

The hierarchical distinction between biomarkers and surrogate endpoints is intended to indicate that relatively few biomarkers will meet the stringent criteria that are needed for them to serve as reliable substitutes for clinical endpoints. In fact, not all clinical endpoints are equally definitive and they can be further categorized as “intermediate endpoint” (a clinical endpoint that is not the ultimate outcome but is nonetheless of real clinical benefit) and “ultimate outcome” (a clinical endpoint such as survival, onset of serious morbidity, or symptomatic response that captures the benefits and risks of an intervention.” In some cases, the clinical benefit of an intermediate endpoint may be important to patients even though this benefit is not associated with improvement in the clinical outcome of increased survival. However, in other cases, when the ultimate outcome is considered, the clinical benefit of an intermediate endpoint is more than offset by the adverse effects of drug therapy.

A high level of stringency is required when a biomarker response is substituted for a clinical outcome and is proposed as the basis for regulatory approval of an application to market a new drug. However, biomarkers need not be validated as rigorously in order to play other important roles, such as facilitating our understanding of disease mechanisms and natural history, expediting the development of new drugs, addressing regulatory concerns related to dose-exposure-response relationships, and even assisting with some aspects of clinical practice.

Thus, arrays of the present invention may be used as a tool of identifying and/or measuring surrogate markers. Specifically, the subject arrays containing a subset of candidate small molecules that might be important biomarkers for certain disease conditions can be used to rapidly profile a large number of disease v. normal samples, such that a pattern of profile changes specific for the disease condition can be readily identified. Consistent and statistically significant changes in profile of certain small molecules are deemed to be associated with such specific disease conditions, and may serve as surrogate markers for such diseases. The arrays of the invention can be used to measure the level or changes thereof for markers of disorders or disease states, for markers for precursors of disease states, for markers for predisposition of disease states, for markers of exposure to toxic agents, for markers of drug activity, or for markers of the pharmacogenomic profile of protein expression and/or profile of metabolites.

Such biomarkers play an important role in the preclinical assessment of potentially beneficial and harmful effects of a new drug candidate. Screening tests in animals using biomarkers provide important demonstration that a compound is likely to have the intended therapeutic activity in patients. Biomarkers for potential toxicity play an equally important role. Biomarkers are perhaps most useful in the early phases of drug development, when measurement of clinical endpoints may be too time-consuming or cumbersome to provide timely proof of concept or dose-ranging information. However, the continued use of such markers may also be very helpful in late stage clinical development. Perhaps the most widespread application of surrogate endpoints in late-phase clinical development is in the substitution of drug concentration measurements for clinical endpoints in the registration of new drug formulations and generic drug products. Federal regulations state that measurement of either blood concentrations or urine excretion rates of a drug may be used to demonstrate that a new formulation has bioavailability comparable to that of the reference material (US Gov. Print. Off. 1997. Code of Federal Regulations, Title 21, Vol. 5, Part 320, Subpart B. Washington, D.C.: US Gov. Print. Off.).

To illustrate, genetic mutations and environmental insults are believed to contribute to the death of neurons. Specific metabolic signatures are starting to emerge for the different subtypes of MND (motor neuron disease). Databases are being established that link biochemical changes with clinical endpoints, the chemical identification of which could highlight disease-related biochemical and signaling events, and diagnostic markers for the diseases. Profiling the metabolites and their change pattern may also be used to screen for potential therapeutic lead molecules.

It is contemplated that either single small molecule or a combination of several small molecules can serve as biomarkers or surrogate endpoints. If a combination of several small molecules are used, only when all small molecules have predicted profile changes can a disease association be implicated. In fact, perhaps the most significant use of the invention is that it enables practice of a powerful new analysis technique: analyses of samples for the presence of specific combinations of proteins/small molecules and specific levels of combinations of proteins/small molecules. This is valuable in molecular biology investigations generally, and particularly in development of novel assays. Thus, this invention permits one to identify analytes (proteins and/or small molecules), groups of analytes, and profiles of analytes present in a sample which are characteristic of some disease, physiologic state, or species identity. Such multiparametric assay protocols may be particularly informative if the analytes being detected are from disconnected or remotely connected pathways. For example, the invention might be used to compare profiles of proteins and/or small molecules metabolites in tissue, urine, or blood from normal patients and cancer patients, and to discover that in the presence of a particular type of cancer a first group of analytes are expressed at a higher level than normal and another group are expressed at a lower level. As another example, the subject arrays might be used to survey analyte levels in various strains of bacteria, to discover patterns of expression which characterize different strains, and to determine which strains are susceptible to which antibiotic. Furthermore, the invention enables production of specialty assay devices comprising arrays or other arrangements of capture agents for detecting specific patterns of specific analytes. Thus, to continue the example, in accordance with the practice of the invention, one can produce a chip which can be exposed to a cell lysate preparation from a patient or a body fluid to reveal the presence or absence or pattern of expression informative that the patient is cancer free, or is suffering from a particular cancer type. Alternatively, one might produce a chip that would be exposed to a sample and read to indicate the species of bacteria in an infection and the antibiotic that will destroy it.

A junction PET is a peptide which spans the region of a protein corresponding to a splice site of the RNA which encodes it. Capture agents designed to bind to a junction PET may be included in such analyses to detect splice variants as well as gene fusions generated by chromosomal rearrangements, e.g., cancer-associated chromosomal rearrangements. Detection of such rearrangements may lead to a diagnosis of a disease, e.g., cancer. It is now becoming apparent that splice variants are common and that mechanisms for controlling RNA splicing have evolved as a control mechanism for various physiological processes. The invention permits detection of expression of proteins encoded by such species, and correlation of the presence of such proteins with disease or abnormality. Examples of cancer-associated chromosomal rearrangements include: translocation t(16;21)(p11; q22) between genes FUS-ERG associated with myeloid leukemia and non-lymphocytic, acute leukemia (see Ichikawa H. et al. (1994) Cancer Res. 54(11):2865-8); translocation t(21;22)(q22; q12) between genes ERG-EWS associated with Ewing's sarcoma and neuroepithelioma (see Kaneko Y. et al. (1997) Genes Chromosomes Cancer 18(3):228-31); translocation t(14;18)(q32; q21) involving the bcl2 gene and associated with follicular lymphoma; and translocations juxtaposing the coding regions of the PAX3 gene on chromosome 2 and the FKHR gene on chromosome 13 associated with alveolar rhabdomyosarcoma (see Barr F. G. et al. (1996) Hum. Mol. Genet. 5:15-21).

For applications in environmental and industrial diagnostics the capture agents are designed such that they bind to one or more PET corresponding to a biowarfare agent (e.g., anthrax, small pox, cholera toxin) and/or one or more PET corresponding to other environmental toxins (Staphylococcus aureus a-toxin, Shiga toxin, cytotoxic necrotizing factor type 1, Escherichia coli heat-stable toxin, and botulinum and tetanus neurotoxins) or allergens. The capture agents may also be designed to bind to one or more PET corresponding to an infectious agent such as a bacterium, a prion, a parasite, or a PET corresponding to a virus (e.g., human immunodeficiency virus-1 (HIV-1), HIV-2, simian immunodeficiency virus (SIV), hepatitis C virus (HCV), hepatitis B virus (HBV), Influenza, Foot and Mouth Disease virus, and Ebola virus).

The following part illustrates the general idea of diagnostic use of the instant invention in one specific setting—serum biomarker assays.

The proteins found in human plasma perform many important functions in the body. Over or under expression of these proteins can thus cause disease directly, or reveal its presence. Studies have shown that complex serum proteomic patterns might reflect the underlying pathological state of an organ such as the ovary (Petricoin et al., Lancet 359: 572-577, 2002). Therefore, the easy accessibility of serum samples, and the fact that serum comprehensively samples the human phenotype—the state of the body at a particular point in time—make serum an attractive option for a broad array of applications, including clinical and diagnostics applications (early detection and diagnosis of disease, monitor disease progression, monitor therapy etc.), discovery applications (such as novel biomarker discovery), and drug development (drug efficacy and toxicity, and personalized medicine). In fact, over $1 billion annually is spent on immunoassays to measure proteins in plasma as indicators of disease (Plasma Proteome Institute (PPI), Washington, D.C.).

Despite decades of research, only a handful of proteins (about 20) among the 500 or so detected proteins in plasma are measured routinely for diagnostic purposes. These include: cardiac proteins (troponins, myoglobin, creatine kinase) as indicators of heart attack; insulin, for management of diabetes; liver enzymes (alanine or aspartate transaminases) as indicators of drug toxicity; and coagulation factors for management of clotting disorders. About 150 proteins in plasma are measured by some laboratory for diagnosis of less common diseases.

In addition, proteins in plasma differ in concentration by at least one billion-fold. For example, serum albumin has a normal concentration range of 35-50 mg/mL (35-50×10⁹ pg/mL) and is measured clinically as an indication of severe liver disease or malnutrition, while interleukin 6 (IL-6) has a normal range of just 0-5 pg/mL, and is measured as a sensitive indicator of inflammation or infection.

Thus, there is a need for reference levels of all serum proteins, and reliable assays for measuring serum protein levels under any conditions. However, standardization of immunoassays for heterogeneous antigens is nearly impossible about 10 years ago (Ekins, Scand J Clin Lab Invest. 205: 33-46, 1991). One of the major obstacle is the apparent need of having identical standard and analyte. This is the case with only a few small peptides. With larger peptides and proteins, the problems tend to become more complicated because biological samples often contain proforms, splice variants, fragments, and complexes of the analyte (Stenman, Clinical Chemistry 47: 815-820, 2001). One such problem is illustrated by measuring serum TGF-beta levels.

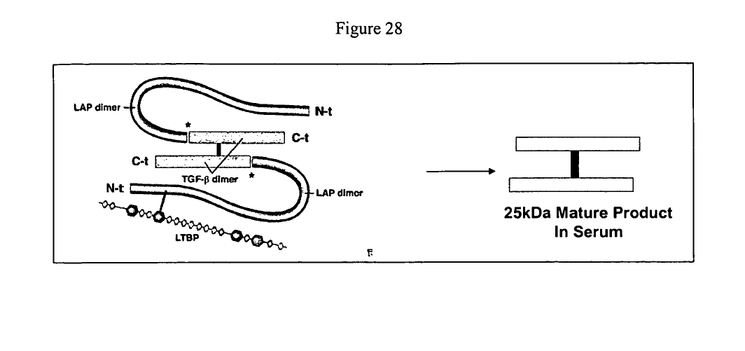

The TGF-beta superfamily proteins are a collection of structurally related multi-function proteins that have a diverse array of biological functions including wound healing, development, oncogenesis, and atherosclerosis. There are at least three known mammalian TGF-beta proteins (beta1, beta2 and beta3), which are thought to have similar functions, at least in vitro. Each of the three isoforms are produced as pre-pro-proteins, which rapidly dimerizes. After the loss of the signal sequences, sugar moieties are added to the proproteins regions known as the Latency Associated Peptide, or LAP. In addition, there is proteolytic cleavage between the LAPs and the mature dimers (the functional portion), but the cleaved LAPs still associate with the mature dimer, forming a complex known as the small latent complex. Either prior to secretion, or in the extracellular milieu, the small latent complex can bind to a large number of other proteins forming a large number of higher molecular weight latent complexes. The best characterized of these proteins are the latent TGF-beta binding protein family LTBP1-4 and fibrillin-1 and -2 (see FIG. 28). Once in the extracellular environment, the TGF-beta complex may bind even more proteins to form other complexes. Known soluble TGF-beta binding proteins include: decorin, alpha-fetoprotein (AFP), betaglycan extracellular domain, β-amyloid precursor, and fetuin. Given the various isoforms, complexes, processing stages, etc., it is very difficult to accurately measure serum TGF-beta protein levels, and a range of 100-fold differences in serum level of TBG-beta1 are reported by different groups (see Grainger et al., Cytokine & Growth Factor Reviews 11: 133-145, 2000).

The other problem arises from the false positive/negative effects of anti-animal antibodies on immunoassays. Specifically, in a sandwich-type assay for a specific antigen in a serum sample, instead of capturing the desired antigen, the immobilized capture antibody may bind to anti-animal antibodies in the serum sample, which in turn can be bound by the labeled secondary antibody and gives rise to false positive result. On the other hand, too much anti-animal antibodies may block the interaction between the capture antibody and the desired antigen, and the interaction between the labeled secondary antibody and the desired antigen, leading to false negative result. This is a serious problem demonstrated in a recent study by Rotmensch and Cole (Lancet 355: 712-715, 2000), which shows that in all 12 cases where women were diagnosed of having postgestational choriocarcinoma on the basis of persistently positive human chorionic gonadotropin (hCG) test results in the absence of pregnancy, a false diagnosis had been made, and most of the women had been subjected to needless surgery or chemotherapy. Such diagnostic problems associated with anti-animal antibodies have also been reported elsewhere (Hennig et al., The influence of naturally occurring heterophilic anti-immunoglobulin antibodies on direct measurement of serum proteins using sandwich ELISAs. Journal of Immunological Methods 235: 71-80, 2000; Covinsky et al., An IgM1 Antibody to Escherichia coli Produces False-Positive Results in Multiple Immunometric Assays. Clinical Chemistry 46: 1157-1161, 2000).

All these problems can be efficiently solved by the methods of the instant invention. By digesting serum samples and converting all forms of the target protein to a uniform PET-containing peptide, the methods of the instant invention greatly reduce the complexity of the sample. Anti-animal antibodies, proteins complexes, various isoforms are no longer expected to be a significant factor in the digested serum sample, thus facilitating more reliable, reproducible, and accurate results from assay to assay.

The method of the instant invention is by no means limited to one particular serum protein such as TGF-beta. It has broad applications in a wide range of serum proteins, including peptide hormones, candidate disease biomarkers (such as PSA, CA125, MMPs, etc.), serum disease and non-disease biomarkers, and acute phase response proteins. For example, measuring the following types of serum biomarkers will have broad applications in clinical and diagnostic uses: 1) disease state markers (such as markers for inflammation, infection, etc.), and 2) non-disease state markers, including markers indicating drug and hormone effects (e.g., alcohol, androgens, anti-epileptics, estrogen, pregnancy, hormone replacement therapy, etc.). Exemplary serum proteins that can be measured include: ApoA-I, Andogens, AAT, AAG, A2M, Alb, Apo-B, AT III, C3, Cp, C4, CRP, SAA, Hp, AGP, Fb, AP, FIB, FER, PAL, PSM, Tf, IgA, IgG, IgM, IgE, FN, B2M, and RBP.

One preferred assay method for these serum proteins is the PET-based peptide competiton assay using immobilized PET peptides, PET-specific capture agents, and at least one labeled secondary capture agent(s) for detection of binding. These assays may be performed in an array format according to the teaching of the instant application, in that different PET-containing peptides can be arrayed on a single (or a few) microarrays for use in simultaneous detection/quantitation of a large number of serum biomarkers.

Foundation for Blood Research (FBR, Scarborough, Me.) has developed a 152page guide on serum protein utility and interpretation for day to day use by practitioners and laboratorians. This guide contains a distillation of the world's literature on the subject, is fully indexed, and is presented by a given disease state (Section I), as well as by individual proteins (Section II). This book is generally useful for interpretation of test results, as well as providing guidance regarding which test is (or is not) appropriate to order and why (or why not). Section II, which covers general information on serum proteins, is also helpful regarding background information about each protein. The entire content of which is incorporated herein by reference.

B. Pharmaceutical Applications

The capture agents or small molecule-based arrays (e.g. PET-based arrays) of the present invention may also be used to study the relationship between a subject's metabolite profile (e.g. protein expression profile) and that subject's response to a foreign compound or drug. Differences in metabolism of therapeutics can lead to severe toxicity or therapeutic failure by altering the relation between dose and blood concentration of the pharmacologically active drug. Thus, use of the capture agents or arrays of the subject invention in the foregoing manner may aid a physician or clinician in determining whether to administer a pharmacologically active drug to a subject, as well as in tailoring the dosage and/or therapeutic regimen of treatment with the drug.

On the other hand, toxicological evaluation of novel compounds requires extensive resources during the development of new pharmaceuticals. In many cases, development of a new compound has to be terminated based on its toxic effects. There is thus a great need for toxicity evaluation assays that can be used earlier in the process of drug development. Identification of markers predictive of toxicity may provide the possibility to screen large numbers of chemicals.

The DNA microarray technology provides information about the transcriptional profile of a sample. The technique has made it possible to survey thousands of genes both for expression monitoring under different physiological conditions and in polymorphism analysis. The usage of gene arrays in toxicology has been termed toxicogenomics.

Quantitative protein expression analysis, as provided by two-dimensional gel electrophoresis (2-DE) followed by identification of individual spots by mass spectrometry (MS), enables the assessment of changes at the level of protein expression (Steiner and Witzmann, Electrophoresis 21:2099-2104, 2000). Fundamental studies have illustrated the usefulness and potential of the proteomic approach to identify changes in rat liver expression profiles associated with the toxicity of compounds (Anderson et al., Toxicol. Pathol. 24:72-76, 1996). Proteomics can also provide essential information for mechanistic toxicology (Aicher et al., Electrophoresis 19:1998-2003, 1998), and it measurements address problems that cannot be approached by gene expression analysis, such as the abundance of a gene product, post-translational modifications, sub-cellular localization as well as interaction with other proteins and functional aspects.

However, neither genomics nor proteomics provide a holistic picture of a toxicological episode. The metabolic status of the whole organism needs to be taken into account in order to increase the understanding of the toxicity of compounds. For example, the application of ¹H-NMR spectroscopy combined with pattern-recognition based methods to biofluid analysis (also called metabonomics) gives rise to a comprehensive metabolic profile of the low molecular weight components of biofluids, e.g. urine (Nicholson et al., Xenobiotica 29:1181-1189, 1999). This metabolic profile reflects concentrations and fluxes of endogenous metabolites and gives an indication of an organism's physiological or pathophysiological status. The rapid progress of these technologies creates a unique opportunity to dramatically improve mechanistic studies as well as the predictive power of toxicological studies.

Historically, measurement of metabolites in human biofluids has been used for the diagnosis of a number of genetic conditions and for assessing exposure to certain xenobiotics. Traditional analysis approaches have focused on one or a few metabolites. The instant invention provides a cheap, efficient, and fast approach as an alternative to the more expensive techniques that rely heavily on advanced instruments.

In general, metabolite profiling may be more advantageous in certain situations, since routine assays for prediction of drug toxicity often result in false positive and false negative findings. In the case of liver toxicants, tests used to evaluate toxicity in vivo assess hepatocyte integrity rather than liver function. Approaches such as gene expression profiling may be non-specific, expensive, and invasive, and may generate only limited information on the precise mechanism(s) of drug action. Metabolic profiling is an important discipline focused on the comprehensive analysis of the low molecular weight biochemicals present in cells, tissues and biofluids. It is an integral part of biological pathways and networks that is “downstream” of the genome and the proteome. Consequently, the metabolome is more directly influenced by external agents such as diet, drugs, disease, and chemicals than either the genome or the proteome. Furthermore, the ability to combine metabolic profiles with other data streams, including histopathology and pathway data, can provide additional information beyond a simple injury signal, and lays the foundation for a mechanism-based, minimally invasive approach to predicting long-term drug safety and human outcomes.

Metabolite and/or protein profiling may also be advantageous over measurement of individual metabolites as is routinely done in standard diagnostic tests. This is because successful therapy for chronic diseases must normalize a targeted aspect of metabolism without disrupting the regulation of other metabolic pathways essential for maintaining health. Use of a limited number of single molecule surrogates for disease, or biomarkers, to monitor the efficacy of a therapy may fail to predict undesirable side effects. For example, in a recent study by Watkins et al. (J Lipid Res. 43(11): 1809-17, 2002), a comprehensive metabolomic assessment of lipid metabolites was employed to determine the specific effects of the peroxisome proliferator-activated receptor gamma (PPARgamma) agonist rosiglitazone on structural lipid metabolism in a new mouse model of Type 2 diabetes. Dietary supplementation with rosiglitazone (200 mg/kg diet) suppressed Type 2 diabetes in obese (NZO×NON)F1 male mice, but chronic treatment markedly exacerbated hepatic steatosis. The metabolomic data revealed that rosiglitazone i) induced hypolipidemia (by dysregulating liver-plasma lipid exchange), ii) induced de novo fatty acid synthesis, iii) decreased the biosynthesis of lipids within the peroxisome, iv) substantially altered free fatty acid and cardiolipin metabolism in heart, and v) elicited an unusual accumulation of polyunsaturated fatty acids within adipose tissue. These observations suggest that the phenotypes induced by rosiglitazone are mediated by multiple tissue-specific metabolic variables. Because many of the effects of rosiglitazone on tissue metabolism were reflected in the plasma lipid metabolome, metabolomics has excellent potential for developing clinical assessments of metabolic response to drug therapy.

For example, Griffin et al. (Anal Biochem. 293(1):16-21, 2001) realized that a principal problem in understanding the functional genomics of a pathology is the wide-reaching biochemical effects that occur when the expression of a given protein is altered. To complement the information available to bioinformatics through genomic and proteomic approaches, Griffin et al. used a novel method of providing metabolite profiles for a disease, using pattern recognition coupled with ¹H NMR spectroscopy. Using this technique, the mdx mouse, a model of Duchenne muscular dystrophy (DMD) was examined. It was found that Dystrophic tissue had distinct metabolic profiles not only for cardiac and other muscle tissues, but also in the cerebral cortex and cerebellum, where the role of dystrophin is still controversial. These metabolic ratios were expressed crudely as biomarker ratios to demonstrate the effectiveness of the approach at separating dystrophic from control tissue (cardiac (taurine/creatine): mdx=2.08+/−0.04, control 1.55+/−0.04, P<0.005; cortex (phosphocholine/taurine): mdx=1.28+/−0.12, control=0.83+/−0.05, P<0.01; cerebellum (glutamate/creatine): mdx=0.49+/−0.03, control=0.34+/−0.03, P<0.01). This technique produced new metabolic biomarkers for following disease progression but also demonstrated that many metabolic pathways are perturbed in dystrophic tissue.

Other research has shown that patients suffering from chronic fatigue and chronic pain disorders can be differentiated from healthy control subjects on the basis of their blood biochemistry and urine excretion profiles. Changes in homeostasis in these patients can be assessed by the measurement of metabolites such as amino acids, organic acids and fatty acids which can be extracted from human body fluids. The measurements of these components comprise metabolic profiles which could then be used to aid the diagnosis of chronic diseases. The types of diseases targeted for investigation would include autism, attention deficit disorder, rheumatoid arthritis, multiple sclerosis, irritable bowel syndrome, schizophrenia, colitis, Tauret's syndrome, Crohn's disease, dyslexia and sleep apnea. Body fluids such as blood (serum) and urine samples would be collected from patients diagnosed by physicians.

For example, recent evidence indicates that serine levels were significantly altered in patients with schizophrenia. Further studies showed that D-serine is a full agonist of the glycine site of the NMDA receptor and when D-serine was added to anti-psychotic regimens, significant improvements in cognitive function were observed with no additional side effects. The production of D-stereo-isomers by bacteria was originally considered as the only biological source of these amino acids. It now appears that racemaze enzymes are produced in the human brain that can convert L-stereo isomers to D-stereo-isomers. Although these D-isomers are not incorporated into proteins, they can exhibit neurotransmitter function. It has been suggested that these D-isomers are then excreted in the urine. The measurement of these isomers in urine, blood, cerebral spinal chord fluid, and animal model samples would therefore provide important information on any anomalies in D-amino acid homeostasis in psychoses.

On the other hand, the metabolome is an integral part of biological pathways and networks that is “downstream” of the genome and the proteome. Consequently, the metabolome is more directly influenced by external agents such as diet, drugs, disease, and chemicals than either the genome or the proteome. The integration of metabolomic with genomic, transcriptomic and/or proteomic data brings together real-world end-points, i.e. actual biological events, with genetic pre-disposition and expression changes. Relating this information to actual phenotypic outcome will provide valuable information on drug toxicity, molecular disease signatures and gene function at several stages in the drug discovery process. The instant invention provides a unique ability to simultaneously monitoring the profiles and changes thereof in both interested metabolites, and the proteome that may be responsible for the levels of these metabolites.

C. Protein Profiling

As indicated above, capture agents or PET-based peptide arrays of the present invention enable the characterization of any biological state via protein profiling. The term “protein profile,” as used herein, includes the pattern of protein expression obtained for a given tissue or cell under a given set of conditions. Such conditions may include, but are not limited to, cellular growth, apoptosis, proliferation, differentiation, transformation, tumorigenesis, metastasis, and carcinogen exposure.

The capture agents or PET-based peptide arrays of the present invention may also be used to compare the protein expression patterns of two cells or different populations of cells. Methods of comparing the protein expression of two cells or populations of cells are particularly useful for the understanding of biological processes. For example, using these methods, the protein expression patterns of identical cells or closely related cells exposed to different conditions can be compared. Most typically, the protein content of one cell or population of cells is compared to the protein content of a control cell or population of cells. As indicated above, one of the cells or populations of cells may be neoplastic and the other cell is not. In another embodiment, one of the two cells or populations of cells being assayed may be infected with a pathogen. Alternatively, one of the two cells or populations of cells has been exposed to a chemical, environmental, or thermal stress and the other cell or population of cells serves as a control. In a further embodiment, one of the cells or populations of cells may be exposed to a drug or a potential drug and its protein expression pattern compared to a control cell.

Such methods of assaying differential protein expression are useful in the identification and validation of new potential drug targets as well as for drug screening. For instance, the capture agents, PET-based peptide arrays, and the methods of the invention may be used to identify a protein which is overexpressed in tumor cells, but not in normal cells. This protein may be a target for drug intervention. Inhibitors to the action of the overexpressed protein can then be developed. Alternatively, antisense strategies to inhibit the overexpression may be developed. In another instance, the protein expression pattern of a cell, or population of cells, which has been exposed to a drug or potential drug can be compared to that of a cell, or population of cells, which has not been exposed to the drug. This comparison will provide insight as to whether the drug has had the desired effect on a target protein (drug efficacy) and whether other proteins of the cell, or population of cells, have also been affected (drug specificity).

The utility of the invention is not limited to diagnosis. The system and methods described herein may also be useful for screening, making prognosis of disease outcomes, and providing treatment modality suggestion based on the profiling of the pathologic cells, prognosis of the outcome of a normal lesion and susceptibility of lesions to malignant transformation.

D. Environmental Applications

It may also be advantageous to detect, quantitate and/or monitor human exposure to certain environmental agents such as toxins or pesticides. Many chemicals break down into harmless metabolites after exposure to sunlight. Many others, however, remain intact until they are processed within the human system where they form metabolites or combine with other elements to form new compounds. Frequently the original pesticide or industrial chemical is not detectable in human samples such as urine, saliva or serum, but one or more metabolites can be detected as markers of the human exposure.

For applications in environmental and industrial diagnostics the capture agents are designed such that they bind to one or more small molecule corresponding to a biowarfare agent (e.g., anthrax, small pox, cholera toxin) and/or one or more small molecule corresponding to other environmental toxins (Staphylococcus aureus α-toxin, Shiga toxin, cytotoxic necrotizing factor type 1, Escherichia coli heat-stable toxin, and botulinum and tetanus neurotoxins) or allergens. The capture agents may also be designed to bind to one or more analytes corresponding to an infectious agent such as a bacterium, a prion, a parasite, or an analyte corresponding to a virus (e.g., human immunodeficiency virus-1 (HIV-1), HIV-2, simian immunodeficiency virus (SIV), hepatitis C virus (HCV), hepatitis B virus (HBV), Influenza, Foot and Mouth Disease virus, and Ebola virus).

The utility of the invention is not limited to diagnosis. The system and methods described herein may also be useful for screening, making prognosis of disease outcomes, and providing treatment modality suggestion based on the profiling of the pathologic cells, prognosis of the outcome of a normal lesion and susceptibility of lesions to malignant transformation.

E. Agricultural Applications

Monitoring Metabolic Changes

The metabolic profile of any crop or microbe can be affected by many parameters, such as environmental conditions, stage of growth, interaction with other species and genetic make-up. Crops that are produced for animal feeds or human consumption can undergo subtle changes in their metabolic profile which often go unnoticed if the metabolites are present in small amounts or are undetected by standard analytical methods

The subject arrays provide an efficient and cost-effective means to measure the detailed metabolic primary and secondary profile of a GM crop and compare it to the profile of the non-GM version of the crop, so that changes due to the genetic modification can be seen. These changes could be beneficial (change in vitamins) or non-beneficial (change in toxin levels). This technology can also be used to monitor differences between organically and non-organically produced crops for animal feeds or human consumption; to analyze microbes used in fermentations and other bioprocesses to examine production of novel or interesting metabolites.

Fingerprinting the Food Chain

Food traceability and quality control issues are of growing concern to both the consumer and industry. Consumers want reassurance that the foods they buy have a guaranteed quality and consistency of content. Producers would like to provide this reassurance to give them the edge in the marketplace and to protect their own interests.

Chemical fingerprinting of human foods, animal feeds and drinks offers a way to provide a detailed, sensitive and comprehensive analysis. This can ensure a high degree of quality control for any product so that its exact chemical composition can be described and monitored. Applications in this field include:
A range of foodstuffs
Juices, alcoholic drinks, teas and oils
Herbal remedies and health food products
   Biowaste Processing

The treatment of biowaste streams from food, feed and drink processes can be a substantial burden on resources. Large volumes of low value material must be processed and disposed of in ever more sustainable fashions. Metabolite profiling with the subject arrays can be used to examine such potential waste materials for novel compounds that could give it added value.

These technologies can be applied across a range of industries from food and drink processing to analysis of agricultural wastes. It may now be possible to convert waste from food processing, or other biomaterials into feedstocks for producing novel high value compounds.

### EXAMPLES

This invention is further illustrated by the following examples which should not be construed as limiting. The contents of all references, patents and published patent applications cited throughout this application, as well as the Figures are hereby incorporated by reference.

### Example 1

### Identification of Proteome Epitope Tags Within the Human Proteome

As any one of the total 20 amino acids could be at one specific position of a peptide, the total possible combination for a tetramer (a peptide containing 4 amino acid residues) is 20⁴; the total possible combination for a pentamer (a peptide containing 5 amino acid residues) is 20⁵ and the total possible combination for a hexamer (a peptide containing 6 amino acid residues) is 20⁶. In order to identify unique recognition sequences within the human proteome, each possible tetramer, pentamer or hexamer was searched against the human proteome (total number: 29,076; Source of human proteome: EBI Ensembl project release v 4.28.1 on Mar. 12, 2002).

The results of this analysis, set forth below, indicate that using a pentamer as a unique recognition sequence, 80.6% (23,446 sequences) of the human proteome have their own unique recognition sequence(s). Using a hexamer as a unique recognition sequence, 89.7% of the human proteome have their own unique recognition sequence(s). In contrast, when a tetramer is used as a unique recognition sequence, only 2.4% of the human proteome have their own unique recognition sequence(s).

Results and Data

2.1. Tetramer Analysis:

2.1.1. Sequence Space:

| | | |
|---|---|---|
| Total number of human protein sequences | 29,076 | 100% |
| *Number of sequences with 1 or more | 684 | 2.4% |
| unique tetramer tag | | |
| Number of sequences with 0 unique | 28,392 | 97.6% |
| tetramer tag | | |

*For these 684 sequences, average Tag/sequence: 1.1.

2.1.2. Tag Space:

| | | |
|---|---|---|
| Total number of tetramers | 20⁴ = 160,000 | 100% |
| Tetramers found in 0 sequence | 393 | 0.2% |
| ^{#}Tetramers found in 1 sequence only | 745 | 0.5% |
| Tetramers found in more than 1 sequences | 158,862 | 99.3% |

^{#}These are signature tetra-peptides

2.2. Pentamer Analysis:

2.2.1. Sequence Space:

| | | |
|---|---|---|
| Total number of human protein sequences | 29,076 | 100% |
| *Number of sequences with 1 or more | 23,446 | 80.6% |
| unique pentamer tag | | |
| Number of sequences with 0 unique | 5,630 | 19.4% |
| pentamer tag | | |

*For these 23,446 sequences, Average Tag/sequence: 23.9

2.2.2. Tag Space:

| | | |
|---|---|---|
| Total number of pentamers | 20⁵ = 3,200,000 | 100% |
| Pentamers found in 0 sequence | 955,007 | 29.8% |
| ^{#}Pentamers found in 1 sequence only | 560,309 | 17.5% |
| Pentamers found in more than 1 sequences | 1,684,684 | 52.6% |

^{#}These are signature penta-peptides

2.3. Hexamer Analysis:

2.3.1. Sequence Space:

| | | |
|---|---|---|
| Total number of human protein sequences | 29,076 | 100% |
| *Number of sequences with 1 or more | 26,069 | 89.7% |
| unique hexamer tag | | |
| Number of sequences with 0 unique | 3,007 | 10.3% |
| hexamer tag | | |

*For these 26069 sequences, Average Tag/sequence: 177

2.3.2. Tag Space:

| | | |
|---|---|---|
| Total number of hexamers | 20⁶ = 64,000,000 | 100% |
| hexamers found in 0 sequence | 57,040,296 | 89.1% |
| ^{#}hexamers found in 1 sequence only | 4,609,172 | 7.2% |
| hexamers found in more than 1 sequences | 2,350,532 | 3.7% |

^{#}These are signature hexa-peptides.

Similar analysis in the human proteome was done for PET sequences of 7-10 amino acids in length, and the results are combinedly summarized in the table below:

| | | Tagged | |
|---|---|---|---|
| | Tagged | Sequences | Average PET |
| PET Length | Sequences | (% of total - | (Number/ |
| (Amino Acids) | (Number) | 29076) | Tagged Protein) |
| 4 | 684 | 2.35% | 3 |
| 5 | 23,446 | 80.64% | 24 |
| 6 | 26,069 | 89.66% | 177 |
| 7 | 26,184 | 90.05% | 254 |
| 8 | 26,216 | 90.16% | 268 |
| 9 | 26,238 | 90.24% | 272 |
| 10 | 26,250 | 90.28% | 275 |

### Example 2

### Identification of Specific Pets

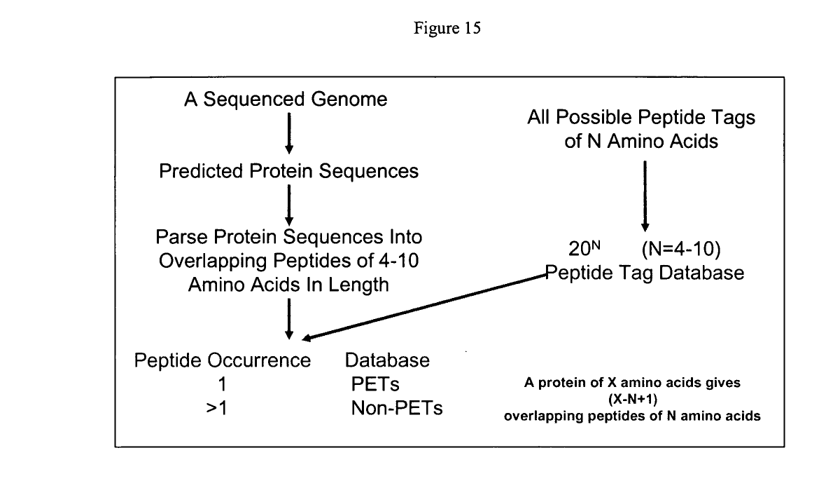

FIG. 15 outlines a general approach to identify all PETs of a given length in an organism with sequenced genome or a sample with known proteome. Briefly, all protein sequences within a sequenced genome can be readily identified using routine bioinformatic tools. These protein sequences are parsed into short overlapping peptides of 4-10 amino acids in length, depending on the desired length of PET. For example, a protein of X amino acids gives (X—N+1) overlapping peptides of N amino acids in length. Theoretically, all possible peptide tags for a given length of, for example, N amino acids, can be represented as 20^{N} (preferably, N=4-10). This is the so-called peptide tag database for this particular length (N) of peptide fragments. By comparing each and every sequence of the parsed short overlapping peptides with the peptide tag database, all PET (with one and only one occurrence in the peptide tag database) can be identified, while all non-PET (with more than one occurrence in the peptide tag database) can be eliminated.

As indicated above, each possible tetramer, pentamer or hexamer was searched against the human proteome (total number: 29,076; Source of human proteome: EBI Ensembl project release 4.28.1 on Mar. 12, 2002, http://www.ensembl.org/Homo_sapiens/) to identify proteome epitope tags (PETs).

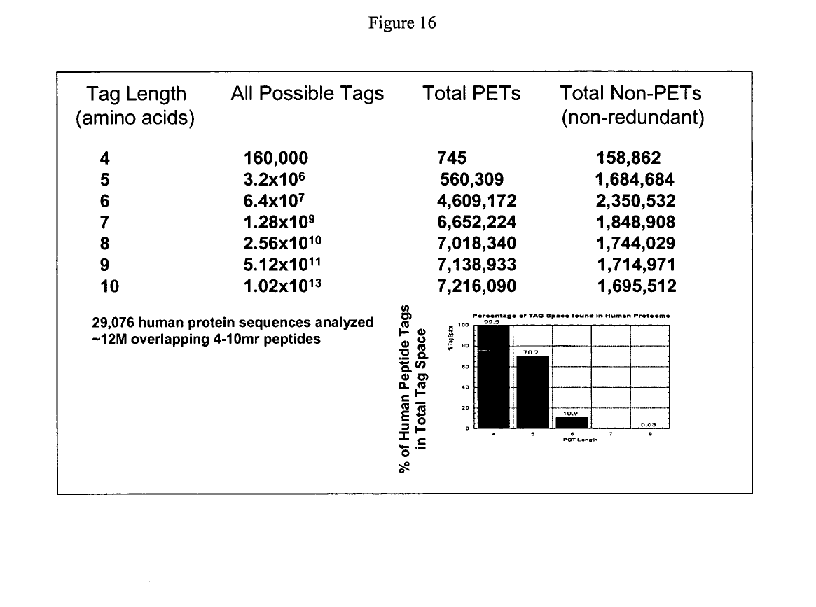

Based on the foregoing searches, specific PETs were identified for the majority of the human proteome. FIG. 16 lists the results of searching the whole human proteome (a total of 29,076 proteins, which correspond to about 12 million 4-10 overlapping peptides) for PETs, and the number of PETs identified for each N between 4-10.

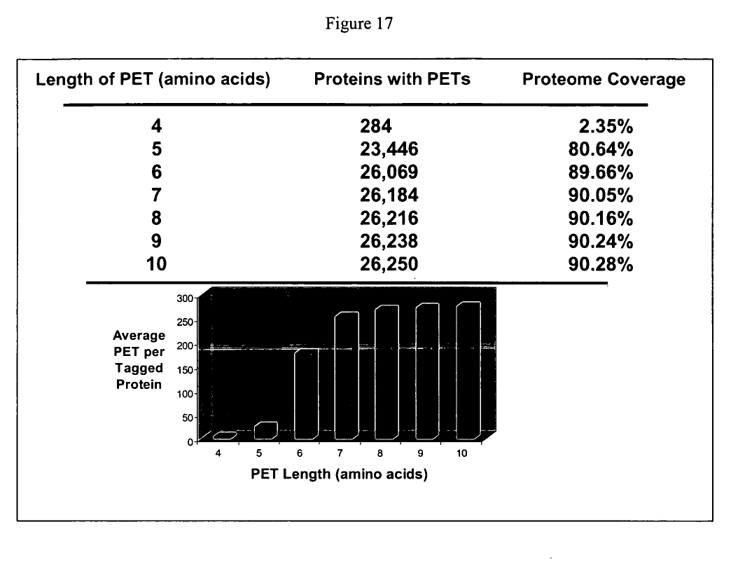

FIG. 17 shows the result of percentage of human proteins that have at least one PET(s). It is shown that for a PET of 4 amino acids in length, only 684 (or about 2.35% of the total human proteins) proteins have at least one 4-mer PETs. However, if PETs of at least 6 amino acids are used, at least about 90% of all proteins have at least one PET. In addition, it is somewhat surprising that there is a significant increase in average number of PETs per protein from 5-mer PETs to 6-mer (or more) PETs (see lower panel of FIG. 17), and that average quickly reaches a platue when 7- or 8-mer PETs are used. These data indicates that PETs of at least 6 amino acids, preferably 7-9 amino acids, most preferably 8 amino acids have the optimal length of PETs for most applications. It is easier to identify a useful PET of that length, partly because of the large average number of PETs per protein when a PET of that length is sought.

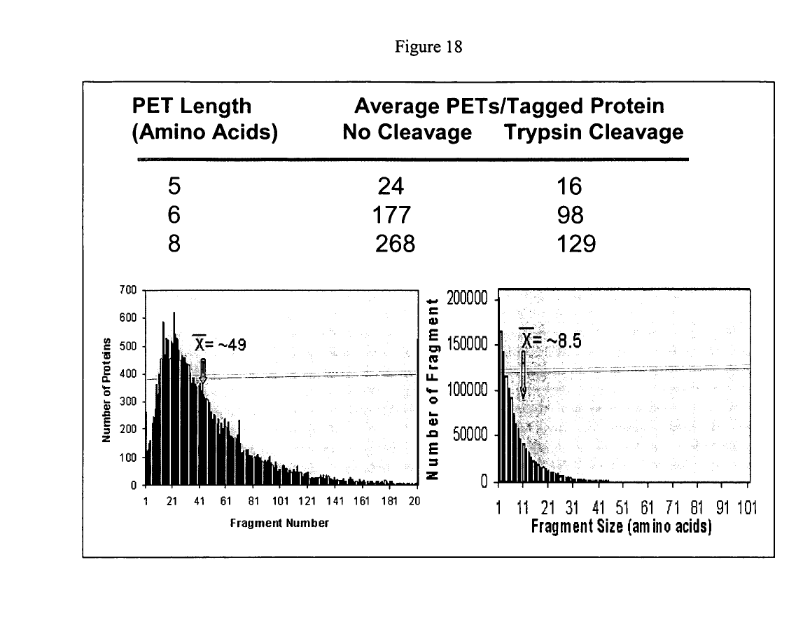

FIG. 18 provides further data resulting from tryptic digest of the human proteome. Specifically, the top panel lists the average number of PETs per tagged protein (protein with at least one PETs), with or without trypsin digestion. Trypsin digestion reduces the average number of PETs per tagged protein by roughly ⅓ to ½. The bottom right panel shows the distribution of tryptic fragments in the human proteome, listed according to peptide length. On average, a typical tryptic fragment is about 8.5 amino acids in length. The bottom left panel shows the distribution of number of tryptic fragments generated from human proteins. On average, a human protein has about 49 tryptic fragments.

### Example 3

### Identification of Sars-Specific Pets

The following example illustrates a general example of identifying organism-specific PET peptides. The same approach and procedures can be used for any other organisms, proteomes, or all the proteins within a specific protein sample.

Sequence Retrieval

A total of 2028 Coronavirus peptide sequences were obtained from the NCBI database (http://www.ncbi.nlm.nih.gov:80/genomes/SARS/SARS.html). These sequences represent at least 10 different species of Coronavirus. Among them, 1098 non-redundant peptide sequences were identified. Each sequence that appeared identically within (was subsumed in) a larger sequence was removed, leaving the larger sequence as the representative. The resulting sequences were then broken up into overlapping regions of eight amino acids (8-mers), with a sequence difference of 1 amino acid between successive 8-mers. These 8-mers were then queried against a database consisting of all 8-mers similarly generated and present in the proteome of the species in question (or any other set of protein sequences deemed necessary). 8-mers found to be present only once (the sequence identified only itself) were considered unique. The remainder of the sequences were initially classified as non-unique with the understanding that with more in-depth analysis, they might actually be as useful as those sequences initially determined to be unique. For example, an 8-mer may be present in another isoform of its parent sequence, so it would still be useful in uniquely detecting that parental sequence and that isoform from all other unrelated proteins.

A total of ˜650,000 8-mer peptide sequences were generated, ˜50,000 of which were determined to be PETs. Among these, 605 were SARS-specific and 602 were PETs relative to human.

PET Prioritization:

Once PETs have been identified, the best candidates for a particular application must be chosen from the pool of all PETs.

Generally, PETs are ranked based upon calculations used to predict their hydrophobicity, antigenicity, and solubility, with hydrophilic, antigenic, and soluble PETs given the highest priority. The PETs are then further ranked by determining each PET's closest nearest neighbors (similar looking 8-mers with at least one sequence difference(s)) in the proteome(s) in question. A matrix calculation is performed using a BLOSUM, PAM, or a similar proprietary matrix to determine sequence similarity and distance. PETs with the most distant nearest neighbors are given the priority.

The parental peptide sequence is then proteolytically cleaved in silico and the resulting fragments sorted by user-defined size/hydrophobicity/antigenicity/solubility criteria. The presence of PETs in each fragment is assessed, and fragments containing no PETs are discarded. The remaining fragments are analyzed in terms of PET placement within them depending upon the requirements of the type of assay to be performed. For example, a sandwich assay prefers two non-overlapping PETs in a single fragment. The ideal final choice would be the most antigenic PETs with only distantly-related nearest neighbors in an acceptable proteolytic fragment that fit the requirements of the assay to be performed.

### Example 4

### Competition Assay

In certain embodiments of the invention, a peptide competition assay may be used to determine the binding specificity of a capture agent towards its target PET, as compared to several nearest neighbor sequences of the PET. The same protocol can be adapted for small molecule-based competition assay

For a typical peptide competition assay, the following illustrative protocol may be used: 1 μg/100 μl/well of each target peptide is coated in Maxisorb Plates with coating buffer (carbonate buffer, pH 9.6) overnight at 4° C., or 1 hour at room temperature. The plates are washed with 300 μl of PBST (1×PBS/0.05% Tween 20) for 4 times. Then 300 μl of blocking buffer (2% BSA/PBST) is added and the plates are incubated for 1 hour at room temperature. Following blocking, the plates are washed with 300 μl of PBST for 4 times.

Synthesized competition peptides are dissolved in water to a final concentration of 2 mM solution. Serial dilution of competition peptides (for example, from 100 pM to 100 μM) in digested human serum are prepared. These competition peptides at particular concentrations are then mixed with equal amounts of primary antibodies against the target peptide. These mixtures are then added to plate wells with immobilized target peptides respectively. Binding is allowed to proceed for 2 hours at room temperature. The plates are washed with 300 μl of PBST for 4 times. Then labeled secondary antibody against the primary antibody, such as 100 μl of 5,000× diluted anti-rabbit-IgG-HRP, is added and incubated for 1 more hour at room temperature. The plates are washed with 300 μl of PBST for 6 times. For detection of the HRP label activity, add 100 μl of TMB substrate (for HRP) and incubate for 15 minutes at room temperature. Add 100 μl of stop buffer (2N HCL) and read the plates at OD₄₅₀. A peptide competition curve is plotted using the ABS at OD₄₅₀ versus the competitor peptide concentrations.

### Example 5

### Pet-Specific Antibodies are Highly Specific and Have High Affinity for their Pet Antigens

There are numerous PET-specific antibodies that were shown to be highly specific and have high affinity for their respective antigens. The following table lists a few exemplary antibodies showing high affinity (low nanomolar to high picomolar range) for their respective antigens.

| | | | |
|---|---|---|---|
| | Length | Affinity K_{D} | |
| Peptide Sequence | (aa) | (nM) | Reference |

| | | | |
|---|---|---|---|
| GATPEDLNQKLAGN | 14 | 1.4 | Cell 91:799, |
| (SEQ ID NO: 1) | | | 1997 |
| CRGTGSYNRSSFESSSG | 17 | 2.8 | JIM 249:253, |
| (SEQ ID NO: 2) | | | 2001 |
| NYRAYATEPHAKKKS | 15 | 0.5 | EJB 267:1819, |
| (SEQ ID NO: 3) | | | 2000 |
| RYDIEAKVTK | 10 | 3.5 | JI 169:6992, |
| (SEQ ID NO: 4) | | | 2002 |
| DRVYIHPF | 8 | 0.5 | JIM 254:147, |
| (SEQ ID NO: 5) | | | 2001 |
| PQSDPSVEPPLS | 12 | 16 | NG 21:163, |
| (SEQ ID NO: 6) | | (a scFv) | 2003 |
| YDVPDYAS (HA tag) | 8 | 2 | engeneOS |
| (SEQ ID NO: 7) | | | |
| MDYKAFDN (FLAG tag) | 8 | 2.3 | engeneOS |
| (SEQ ID NO: 8) | | | |
| HHHHH (HIS tag) | 5 | 25 | Novagen |
| (SEQ ID NO: 9) | | | |

Further more, the table below shows three additional PET-specific antibodies with similar nanomolar-range affinity for the respective antigens:

| | Ab | | Parental |
|---|---|---|---|
| PET Sequence | name | Affinity (K_{D} in nM) | Protein |
| EPAELTDA | P1 | 5 | PSA |
| (SEQ ID NO: 10) | | | |
| YEVQGEVF | C1 | 31 | CRP |
| (SEQ ID NO: 11) | | | |
| GYSIFSYA | C2 | 200 | CRP |
| (SEQ ID NO: 12) | | | |

These PETs are selected based on the criteria set forth in the instant specification, including nearest neighbor analysis. Listed below are several nearest neighbors of two of the PETs above. These sequences are represented, from top to bottom, in SEQ ID NOs: 13-24, respectively.

| PET | LSEPAELTDAVK | AA Differences |
|---|---|---|
| NNP1 | DEPVELTSAPTGHTFS | 2 |
| NNP2 | AGEAAELQDAEVESSAK | 2 |
| NNP3 | LQEPAELVESDGVPK | 3 |
| NNP4 | AQPAELVDSSGW | 3 |
| NNP5 | GLDPTQLTDALTQR | 3 |
| PET | YEVQGEVFTK | AA Differences |
| NNP1 | HVEVNGEVFQK | 2 |
| NNP2 | SYEVLGEEFDR | 2 |
| NNP3 | QYAVSGEIFVVDR | 3 |
| NNP4 | VYEEQGEIILK | 3 |
| NNP5 | LYEVRGETYLK | 3 |

PET-specific antibodies are not only high affinity antibodies, but also highly specific antibodies showing little, if any cross-reactivity with other closely related peptide sequences.

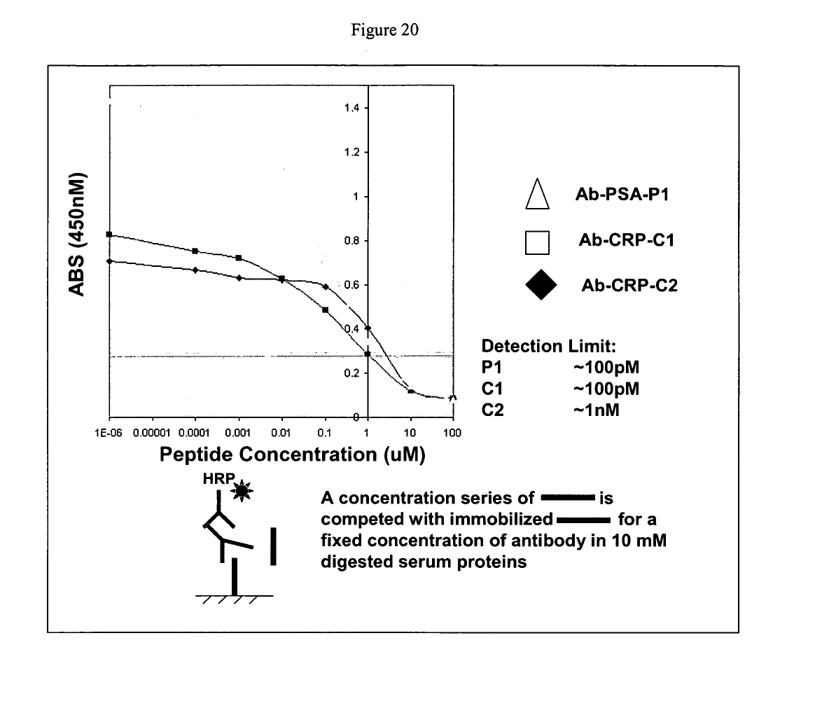

For example, FIG. 20 shows peptide competition results using the peptide competition assay described in Example 5. The left panel shows that antibody P1, which is specific for the PSA-derived 8-mer PET sequence EPAELTDA (SEQ ID NO: 10), can be effectively competed away by the antigen PET (EPAELTDA, SEQ ID NO: 10), with a half-maximum effective peptide concentration of around 40 nM. However, two of its nearest-neighbor 8-mer PETs found in the human proteome with only two- or three-amino-acid differences, EPVELTSA (SEQ ID NO: 25) and DPTQLTDA (SEQ ID NO: 26), are completely ineffective even at 1000 μM (25,000-fold higher concentration). Similarly, the right panel shows that antibody C1, which is specific for the CRP-derived 8-mer PET sequence YEVQGEVF (SEQ ID NO: 11), can be effectively competed away by the antigen PET sequence YEVQGEVF (SEQ ID NO: 11), with a half-maximum effective peptide concentration of around 1 μM. However, two of its nearest-neighbor 8-mer PETs found in the human proteome with only two-amino-acid differences, VEVNGEVF (SEQ ID NO: 27) and YEVLGEEF (SEQ ID NO: 28), are completely ineffective even at 1000 μM (at least 1,000-fold higher concentration).

### Example 6

### Antibody Cross-Reactivity: Kallikrein Ab's

The kallikreins are a subfamily of the serine protease enzyme family (Bhoola et al., Pharmacol Rev 44: 1-80, 1992; Clements J. The molecular biology of the kallikreins and their roles in inflammation. Farmer S. eds. The kinin system 1997: 71-97 Academic Press New York). The human kallikrein gene family was, until recently, thought to include only three members: KLK1, which encodes for pancreatic/renal kallikrein (hK1); KLK2, which encodes for human glandular kallikrein 2 (hK2); and KLK3, which encodes for prostate-specific antigen (PSA; hK3) (Riegman et al., Genomics 14: 6-11, 1992). The best known of the three classic human kallikreins is PSA, an important biomarker for prostate cancer diagnosis and monitoring. Recently, new serine proteases with high degrees of homology to the three classic kallikreins were cloned. These newly identified serine proteases have now been included in the expanded human kallikrein gene family. The entire human kallikrein gene locus on chromosome 19q13.4 now includes 15 genes, designated KLK1-KLK15; their respective proteins are known as hK1-hK15 (Diamandis et al., Clin Chem 46: 1855-1858, 2000).

KLK13, previously known as KLK-L4, is one of the newly identified kallikrein genes. The protein has 47% and 45% sequence identity with PSA and hK2, respectively (Yousef et al., J Biol Chem 275: 11891-11898, 2000). At the mRNA level, KLK13 expression is highest in the mammary gland, prostate, testis, and salivary glands (Yousef, supra). Although the function of KLK13 is still unknown, KLK13, like all other members of the human kallikrein family, is predicted to encode a secreted serine protease that is likely present in biological fluids. Given the prominent role of PSA as a cancer biomarker and the recent demonstration that other members of this gene family are also potential cancer biomarkers (Diamandis et al., Clin Biochem 33: 369-375, 2000; Luo et al., Clin Chem 47: 237-246, 2001; Diamandis et al., Clin Biochem 33: 579-583, 2000; Luo et al., Clin Chim Acta 7: 806-811, 2001; Diamandis et al., Cancer Res 62: 293-300, 2002), hK13 may also have utility as a disease biomarker. In order to develop a suitable method for measuring hK13 protein in biological fluids and tissues with high sensitivity and specificity, and to further investigate the diagnostic and other clinical applications of this protein, Kapadia et al. (Clinical Chemistry 49: 77-86, 2003) cloned and expressed the full-length recombinant human KLK13 in a yeast expression system, and raised KLK13-specific monoclonal and polyclonal antibodies. A sandwich-type assay revealed that the KLK13 antibody is quite specific—recombinant hK1, hK2, hK3, hK4, hK5, hK6, hK7, hK8, hK9, hK10, hK11, hK12, hK14, and hK15 proteins did not produce measurable readings, even at concentrations 1000-fold higher than that of hK13.

However, it should be noted that this type of antibody specificity defined by cross-reactivity to other related proteins, without any epitope information, can frequently be misleading, and thus the data presented in Kapadia et al. should be interpreted with caution. For one thing, unrelated proteins may have higher sequence homology or conformation similarity than family proteins. It may be pure luck that any hK13 antibody does not cross-react with other highly related family members. However, there is no guarantee that the specific epitope recognized by the hK13 antibody does not appear in other proteins, such as an un-identified kallikrein family member, or an alternative splicing form of hK13. Therefore, antibody specificity is better defined by reactivity to peptides most homologous to a selected PET (nearest neighbor peptides). Antibody cross-reactivity is now readily measurable using peptide competitive assays at a wide dynamic range.

On the other hand, in certain situations, detection for the whole protein family or a specific subset of the family are needed. For example, it has already been demonstrated that multiple kallikreins are overexpressed in ovarian carcinoma (reviewed in Yousef and Diamandis, Minerva Endocrinol 27: 157-166, 2002). There is experimental evidence that these kallikreins may form a cascade enzymatic pathway similar to the pathways of coagulation and fibrinolysis. Therefore, one single antibody specific for the subset of ovarian carcinoma-associated kallikreins is of particular interest in clinical setting. Lastly, the concentrations of competitors used is limited in Kapadia's assay.

These problems can be readily tackled with the approach of the instant invention. For example, the table below lists a common PET for hK1-hK11 (except hK6 and 7, which have their common PETs), as well as PETs specific for each hK proteins listed. In addition, both the family-specific PET and the protein-specific PET are within the same tryptic fragment.

| | |
|---|---|
| hK1 | +TL,22 |
| HSQPWQVAVYSHGWAHCGGVLVHR | (SEQ ID NO: 29) |
| hK2 | |
| IVGGWECEQHSQPWQAALYHFSTFQCGGILVHK | (SEQ ID NO: 30) |
| hK3 | |
| GSQPWGVSLFNGLSFHCAGVLVDR | (SEQ ID NO: 31) |
| hK4 | |
| NSQPWQVGLFEGTSLR | (SEQ ID NO: 32) |
| hK5 | |
| HECQPHSQPWQAALFQGQQLLCGGVLVGR | (SEQ ID NO: 33) |
| hK8 | |
| EDCSPHSQPWQAALVMENELFSCGVLVHR | (SEQ ID NO: 34) |
| hK9 | |
| VLNTNGTSGFLPGGYTCFPHSQPWQAALLVQGR | (SEQ ID NO: 35) |
| hK10 | |
| LLEGDECAPHSQPWQVALYER | (SEQ ID NO: 36) |
| hK11 | |
| PNSQPWQAGLFHLTR | (SEQ ID NO: 37) |
| hK6 | |
| CVTAGTSCLISGWGSTSSPQLR | (SEQ ID NO: 38) |
| Hk7 | |
| VMDLPTQEPALGTTCYASGWGSIEPEEFLTPK | (SEQ ID NO: 39) |

By using these family- and individual-specific PET antibodies (or other suitable capture reagents), the same tryptic digestion can be used for a PET-based peptide competition assay to measure the total concentration of all tryptic peptides sharing the same common PET sequence (using the family-specific PET antibodies). Optionally, selective detection/quantitation of specific family members can also be measured using, for example, individual-PET sequence specific antibodies.

In addition, the same approach may be used to detect the presence of alternative splicing isoforms of any protein. For example, there are three alternative splicing forms of hK15 (* represents trypsin digestion sites):

hK15-V1

(SEQ ID NO: 40)

R*LNPQVR*PAVLPTR*CPHPGEACVVSGWGLVSHEPGTAGSPR*SQG
hK15-V2

(SEQ ID NO: 41)

R*LNPQ--------------------------------------
hK15-V3

(SEQ ID NO: 42)

R*LNPQGDSGGPLVCGGILQGIVSWGDVPCDNTTK*PGVYTK

Thus, SGWGLVSH (SEQ ID NO: 43) is a PET for detecting V1, with the three nearest neighbor peptides being AGWGIVNH (SEQ ID NO: 44), SGWGITNH (SEQ ID NO: 45), and SGWGMVTE (SEQ ID NO: 46). Similarly, WGDVPCDN (SEQ ID NO: 47) is a PET for detecting Vi, with the three nearest neighbor peptides being WKDVPCED (SEQ ID NO: 48), WNDAPCDS (SEQ ID NO: 49), and WNDAPCDK (SEQ ID NO: 50). By immobilizing one or more of the junction PETs, antibodies specific for these junction PETs can be used in peptide competition assays to quantitate the amount of splicing variants in any digested samples.

### Example 7

### Detecting Serum Protein Levels

Due to the fundamental problems in measuring an antigen which exists in more than one form and/or present in different complexes, it may be difficult to reach a consensus on the level of total a serum protein (such as TGF-b1 protein) in normal human plasma. The instant invention provides a method that efficiently solves these problems.

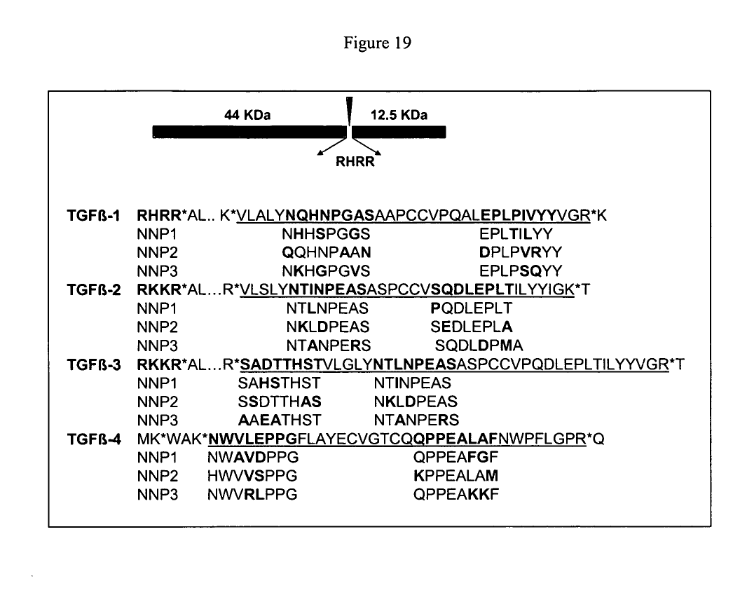

FIG. 19 shows a design for the PET-based assay for standardized serum TGF-beta measurement. The C-terminal monomer for the mature TGF-beta is represented in the top panel as a red bar. The sequences below indicates the PETs specific for each of the 4 TGF-beta isoforms and their respective nearest neighbors. The PET-based peptide competition assay can be used to specifically detect/quantitate one of the TGF-beta isoforms, as well as the total amount of all TGF-beta isoforms present in a serum sample.

### Example 8

### Pet-Based Peptide Competition Assay

FIG. 20 illustrates the results of a PET-based peptide competition assay for three representative PET-peptides, PSA-P1, CRP-C1 and CRP-C2. Briefly, a concentration series of one of the three PET-peptides are used as competitor peptides to compete binding with the identical but immobilized PET peptides, in reaction mixtures with fixed concentration of PET-specific antibodies. The reaction mixture contains 10 mM of digested serum proteins as background. It is evident that the detection limit for the three tested peptides are around 0.1-1 nM.

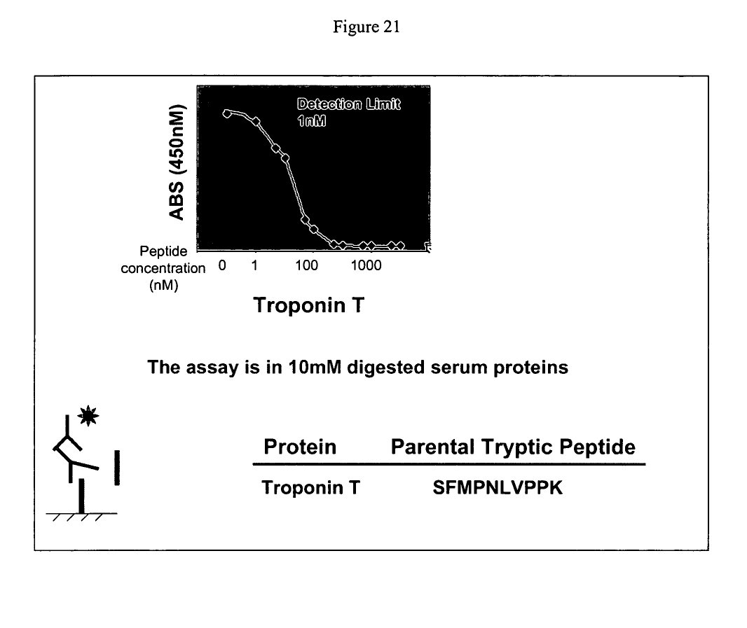

FIG. 21 illustrates a similar assay using a different PET-peptide (SFMPNLVPPK, SEQ ID NO: 51) representing Troponin T. Again, the detection limit is around 1 nM in the 10 mM digested serum protein background.

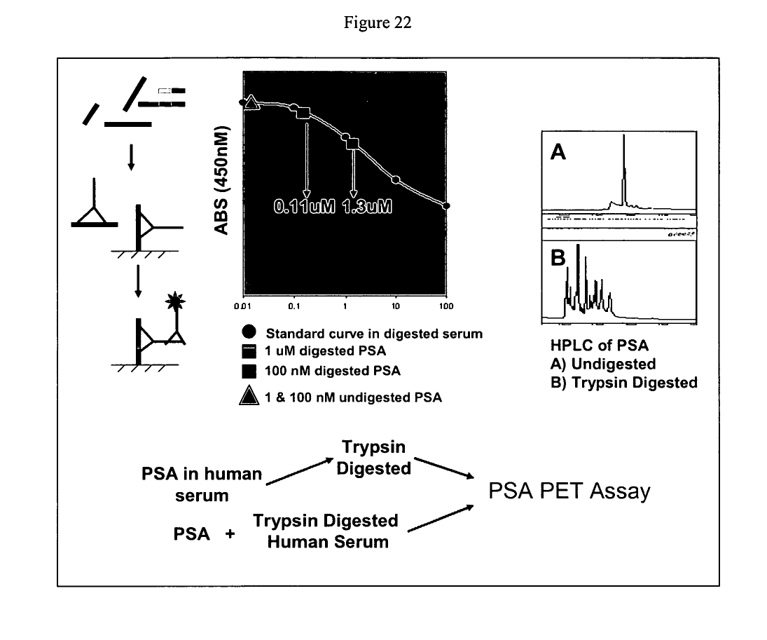

FIG. 22 illustrates that the sample treatment method of the instant invention plays an important role in accurate quantitation of serum protein concentration. For example, if the target peptide PSA is included in human serum before trypsin digestion, the PSA will be digested with all other serum proteins (the HPLC data indicated the completeness of trypsin digestion of PSA since the single PSA peak in the undigested sample was completely replaced by a series of smaller peaks in the trypsin digested sample). As a consequence, the amount measured by the PET-based peptide assay was fairly close to the known value (0.11 uM and 1.3 uM measured as compared to 0.1 uM and 1 uM added, respectively). However, if PSA was directly added as an undigested protein to the trypsin digested serum sample, the measured concentration was quite different from the true values—both much smaller than the true values and there was no significant differences in measured values.

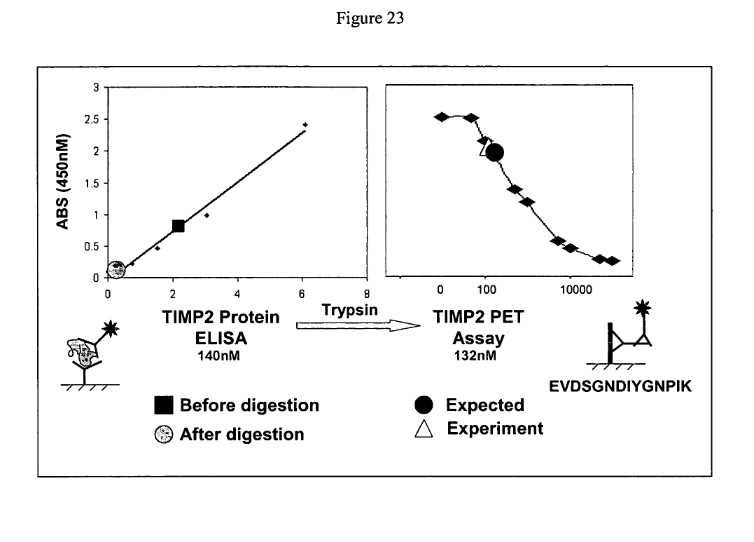

FIG. 23 illustrates that the sample treatment method of the instant invention does not cause appreciatable loss of target proteins in the original sample. The left side of the figure shows the result of a traditional sanwich ELISA assay using a TIMP2-specific antibody. The measured concentration of TIMP2 was about 140 nM. However, after trypsin digestion, there is no measurable TIMP2 using the same ELISA method, demonstrating the completeness of the digestion, and the inability of the primary capture antibody to recognize digested target protein TIMP2. However, the digested peptide fragments can be readily measured by the PET-based peptide competition assay. By using a different antibody specific for a PET within the fragment EVDSGNDIYGNPIK (SEQ ID NO: 52), the measured TIMP2 concentration is about 132 nM, which was essentialy identical to the ELISA result within the errors of measurement.

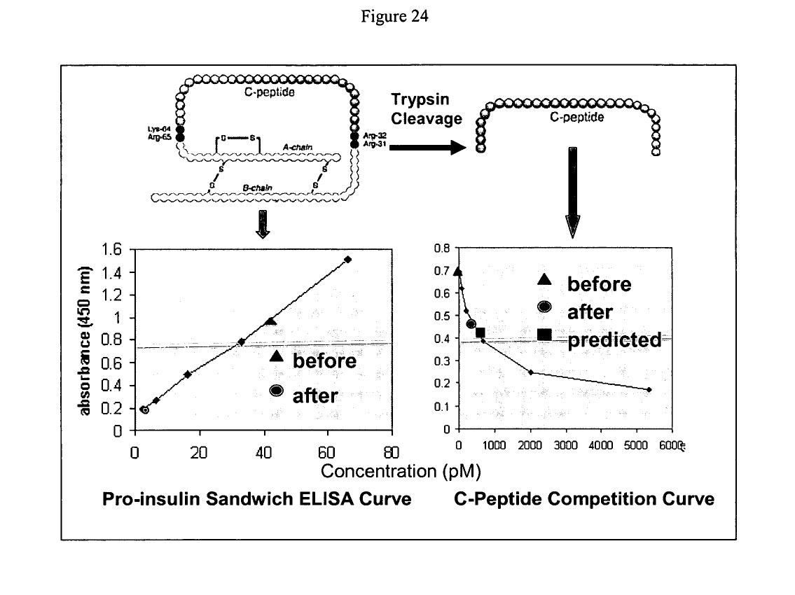

Similar results are obtained using the C-peptide (FIG. 24).

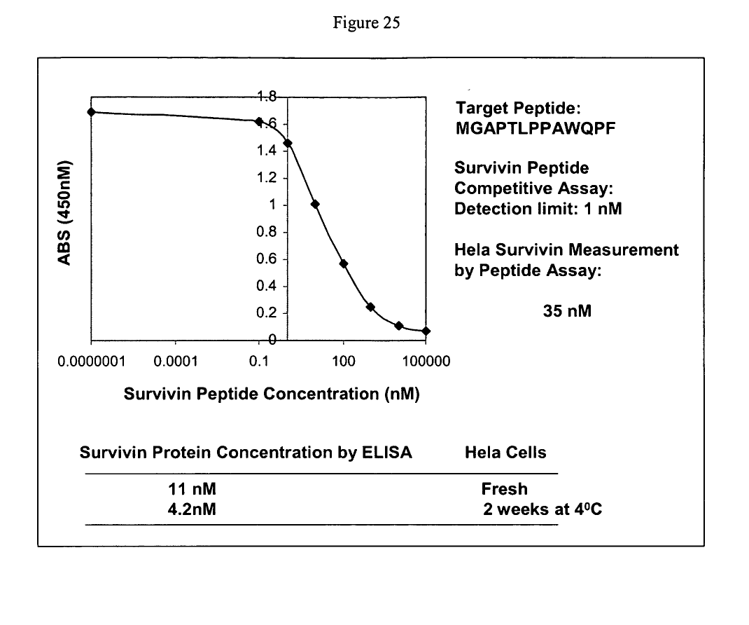

The PET-based peptide competition assay may be used for cell lysates. For example, FIG. 25 indicated that, if the Survivin peptide MGAPTLPPAWQPF (SEQ ID NO: 53) was used as the PET-containing peptide, a detection limit of 1 nM can be achieved based on the standard curve. The concentraton of Survivin in digested Hela cell lysate is about 35 nM. Similar measurement using ELISA, however, only detects a much lower concentration of about 11 nM in fresh Hela cell lysate.

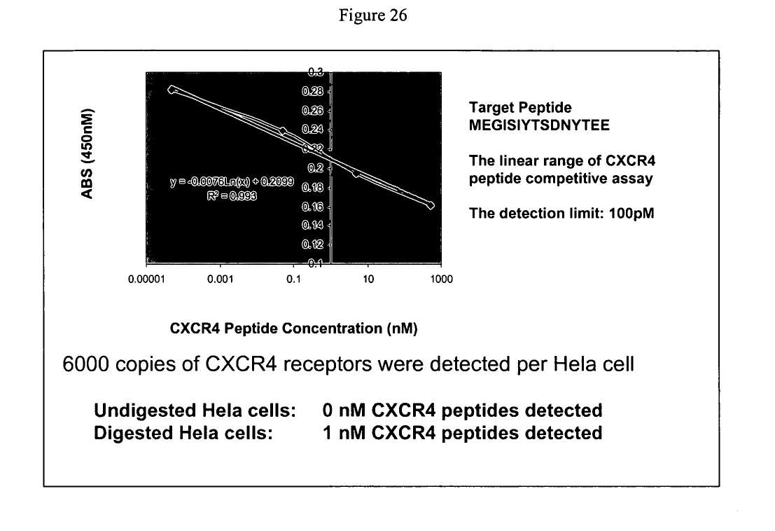

The PET-based peptide competition assay may also be used for membrane proteins. For example, FIG. 26 indicated that, if the CXCR4 membrane protein peptide MEGISIYTSDNYTEE (SEQ ID NO: 54) was used as the PET-containing peptide, a detection limit of 0.1 nM can be achieved based on the standard curve. The concentration of CXCR4 in digested Hela cell lysate is about 1 nM. If the sample was undigested, however, no CXCR4 proteins can be detected in the Hela cell lysate, presumably due to the unavailability of the membrane protein for antibody binding.

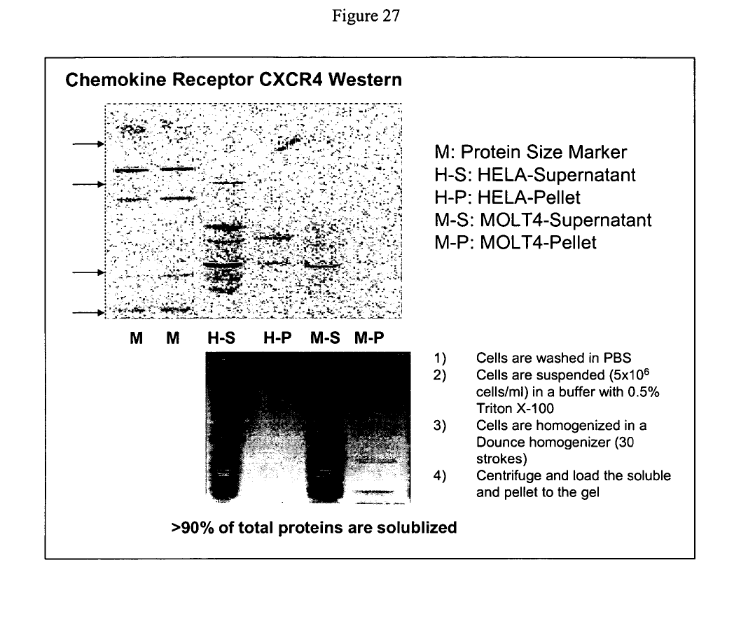

FIG. 27 illustrates the result of extraction of intracellular and membrane proteins. Briefly, cells were washed in PBS, then suspended (5×10⁶ cells/ml) in a buffer with 0.5% Triton X-100 and homogenized in a Dounce homogenizer (30 strokes). The homogenized cells were centrifuged to separate the soluble portion and the pellet, which were both loaded to the gel.

This CXCR4 result also demonstrates that the PET-based peptide assay may be used to detect the presence of very low abundance proteins. If it can be assumed that about 5 million cells are collected in 1 mL, PET-based competition assay can detect as low as 10-100 pM of proteins, which is about 1,000-10,000 molecules/cell.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

Equivalents

A skilled artisan will recognize, or be able to ascertain using no more than routine experimentation, many equivalents to the specific embodiments of the invention described herein. Such equivalents are intended to be encompassed by the following claims.

1. A method for quantitating a plurality of target analytes in a sample, comprising:
(1) immobilizing said plurality of target analytes and/or unique derivatives thereof to a support, said unique derivatives, if used, predictably result from a treatment of said plurality of target analytes within said sample; wherein each of said plurality of target analytes or unique derivatives thereof is immobilized on a series of distinct addressable locations on said support;
(2) for each of said plurality of target analytes or unique derivatives thereof, generating one or more capture agents that specifically bind said target analytes or said unique derivatives thereof;
(3) optionally, subjecting said sample to said treatment;
(4) contacting said plurality of target analytes or unique derivatives thereof on said support to a series of control samples, each within one of the series of distinct addresable locations, and each comprising a mixture of a fixed concentration of said capture agents and a variable concentration of said target analytes or unique derivatives thereof in solution;
(5) generating a standard competition curve for each said plurality of taregt analytes, by measuring the amount of said capture agents bound to said target analytes or unique derivatives thereof on said support;
(6) contacting said plurality of target analytes or unique derivatives thereof on said support to a mixture of said fixed concentration of said capture agent and said sample, in one of the series of distinct addressable locations, optionally after said treatment in step (3);
(7) determining the concentration of each said plurality of target analytes, using each of said standard competition curves, by measuring the amount of said capture agent bound to said target analytes or unique derivatives thereof on said support.

2. The method of claim 1, wherein said plurality of target analytes or derivatives thereof include 5, 10, 20, 50, 100, 500, 1000, 2000, 5000, 10000 or more members.

3. The method of claim 1, wherein in step (1), said plurality of target analytes or derivatives thereof are each immobilized on more than one areas of said series of distinct addressable locations.

4. The method of claim 3, wherein each of said more than one areas contains a different amount of immobilized said target analytes or derivatives thereof.

5. The method of claim 1, wherein said target analytes are small molecules, each independently of molecular weights of about 50-5000 Da, 504000 Da, 50-3000 Da, 50-2000 Da, 50-1000 Da, 50-500 Da, 50-200 Da, or 50-100 Da.

6. The method of claim 5, wherein said small molecules comprises metabolites.

7. The method of claim 6, wherein said metabolites are surrogate markers or potential surrogate markers of a disease or a condition.

8. The method of claim 7, wherein said disease is multiple sclerosis (MS), rheumatoid arthritis (RA), a neoplastic disease, a cardiovascular disease, a neurodegenerative disease, a renal disease, or a hepatic disease.

9. The method of claim 7, wherein said condition is exposure to one or more of: toxic agent selected from: pesticide, environmental toxin, or bacterial toxin; drug candidate; nutritional agent; or allergen.

10. The method of claim 1, wherein said target analyte is a protein, said derivative is a PET sequence of said protein.

11. The method of claim 10, wherein said PET sequence is identified by computationally analyzing amino acid sequence of said target analyte, including a Nearest-Neighbor Analysis that identifies unique amino acid sequences based on criteria that also include one or more of: pI, charge, steric, solubility, hydrophobicity, polarity and solvent exposed area.

12. The method of claim 1, wherein said plurality of target analytes comprise both small molecule and protein.

13. The method of claim 12, wherein said small molecule and protein are surrogate markers or potential surrogate markers of a disease or a condition.

14. The method of claim 13, wherein said disease is selected from multiple sclerosis (MS), rheumatoid arthritis (RA), a neoplastic disease, a cardiovascular disease, a neurodegenerative disease, a renal disease, or a hepatic disease.

15. The method of claim 1, further comprising determining the specificity of each of said capture agent generated in (2) against one or more structurally similar analogs (e.g., nearest neighbors), if any, of said target analyte.

16. The method of claim 15, wherein competition assay is used in determining the specificity of said capture agent generated in (2) against said structurally similar analogs.

17. The method of claim 1, further comprising determining the specificity of each of said capture agent generated in (2) using a proteome matrix array.

18. The method of claim 17, wherein said proteome matrix array comprises polypeptides representing each and every protein wthin the sample.

19. The method of claim 17, wherein said proteome matrix array comprises polypeptides representing the top 100, 300, 500, or 1000 most abundantly expressed proteins within the sample.

20. The method of claim 17, wherein said proteome matrix array excludes excessively hydrophobic peptides, short peptides of no more than 5 residues, or long peptides of no less than 50 residues.

21. The method of claim 17, wherein all peptides on said proteome matrix array have the same concentration.

22. The method of claim 17, wherein each peptide on said proteome matrix array has a concentration proportional to its concentration in the sample.

23. The method of claim 1, wherein the specificity value S for at least 50% of all of said capture agents is no more than about 0.1, preferably no more than about 0.05, 0.02, or 0.01.

24. The method of claim 0.1, wherein said capture agent is a full-length antibody, or a functional antibody fragment selected from: an Fab fragment, an F(ab′)₂ fragment, an Fd fragment, an Fv fragment, a dAb fragment, an isolated complementary determining region (CDR), a single chain antibody (scFv), or derivative thereof.

25. The method of claim 1, wherein said capture agent is a polynucleotide; a PNA (peptide nucleic acid); a protein; a polypeptide; a carbohydrate; an artificial polymer; or a small organic molecule.

26. The method of claim 1, wherein said capture agent is aptamers, scaffolded peptides, or small organic molecules.

27. The method of claim 1, wherein said treatment is denaturation and/or fragmentation of said sample by a protease, a chemical agent, physical shearing, or sonication.

28. The method of claim 27, wherein said denaturation is thermo-denaturation or chemical denaturation.

29. The method of claim 28, wherein said thermo-denaturation is followed by or concurrent with proteolysis using thermo-stable proteases.

30. The method of claim 28, wherein said thermo-denaturation comprises two or more cycles of thermo-denaturation followed by protease digestion.

31. The method of claim 27, wherein said fragmentation is carried out by a protease selected from trypsin, chymotrypsin, pepsin, papain, carboxypeptidase, calpain, subtilis in, gluc-C, endo lys-C, or proteinase K.

32. The method of claim 31, wherein said protease is immobilized on a solid support.

33. The method of claim 1, wherein said sample is a body fluid selected from: saliva, mucous, sweat, whole blood, serum, urine, amniotic fluid, genital fluid, fecal material, marrow, plasma, spinal fluid, pericardial fluid, gastric fluid, abdominal fluid, peritoneal fluid, pleural fluid, synovial fluid, cyst fluid, cerebrospinal fluid, lung lavage fluid, lymphatic fluid, tears, prostatitc fluid, extraction from other body parts, or secretion from other glands; or from supernatant, whole cell lysate, or cell fraction obtained by lysis and fractionation of cellular material, extract or fraction of cells obtained directly from a biological entity or cells grown in an artificial environment.

34. The method of claim 1, wherein said sample is obtained from human, mouse, rat, dog, monkey or other non-human primates, frog (Xenopus), fish (zebra fish), fly (Drosophila melanogaster), nematode (C. elegans), fission or budding yeast, or plant (Arabidopsis thaliana).

35. The method of claim 1, wherein said sample is produced by treatment of membrane bound proteins.

36. The method of claim 1, wherein said capture agent is optimized for selectivity for said analyte or derivative thereof under denaturing conditions.

37. The method of claim 1, wherein the amount of capture agents measured in steps (5) and (7), are independently effectuated by using a secondary agent specific for said capture agent, wherein said secondary agent is labeled by a detectable moiety selected from: an enzyme, a fluorescent label, a stainable dye, a chemilluminescent compound, a colloidal particle, a radioactive isotope, a near-infrared dye, a DNA dendrimer, a water-soluble quantum dot, a latex bead, a selenium particle, or a europium nanoparticle.

38. The method of claim 37, wherein said secondary agent is an antibody labeled by an enzyme or a fluorescent group.

39. The method of claim 1, wherein said analyte or derivative thereof is synthesized on said support.

40. The method of claim 1, wherein said analyte or derivative thereof is synthesized or purified before being immobilized on said support.

41. The method of claim 1; wherein step (2) is effectuated by immunizing an animal with an antigen comprising said analyte or derivative thereof.

42. The method of claim 41, wherein said derivative is a PET sequence, and the N- or C-terminus, or both, of said PET sequence are blocked to eliminate free N- or C-terminus, or both.

43. The method of claim 42, wherein the N- or C-terminus of said PET sequence are blocked by fusing the PET sequence to a heterologous carrier polypeptide, or blocked by a small chemical group.

44. The method of claim 43; wherein said carrier is KLH or BSA.

45. The method of claim 10, wherein said computationally analyzing amino acid sequence includes a solubility analysis that identifies unique amino acid sequences that are predicted to have at least a threshold solubility under a designated solution condition.

46. The method of claim 10, wherein said PET is 5-10 amino acids long.

47. An array for detecting, profiling or quantitating a plurality of target analytes in a sample, said array comprising a plurality of immobilized target analytes or derivatives thereof on a support, each of said plurality of target analytes is represented by at least one of said plurality of immobilized target analytes or derivatives thereof, said derivatives, if present, predictably result from a treatment of said sample, and each of said plurality of peptide fragments contains a PET unique to said fragments within said sample.

48. A method for characterizing a plurality of candidate antibodies for binding affinity, the method comprising:
(1) generating a high density array comprising a plurality of assay chambers, each said chambers contains a plurality of antigens for which said plurality of candidate antibodies are specific, each said antigens are immobilized in said chambers in an addressable location;
(2) contacting each said chamber with a solution of said plurality of candidate antibodies;
(3) determining the affinity of each of said plurality of candidate antibodies for their respective immobilized antigens by measuring the amount of each of said plurality of candidate antibodies bound to said chamber.

49. The method of claim 48, wherein each of said antigens contains a PET.

50. The method of claim 48, wherein each of said antigens is a small molecule metabolite.

51. The method of claim 49 or 50, wherein each of said chamber has 5, 10, 20, 50, 100, or more distinct antigens.

52. The method of claim 48, wherein said solution of said plurality of candidate antibodies contains less than the total numbers of said plurality of peptide antigens in said chamber.

53. The method of claim 48, wherein each said chamber contains the same number of said antigens.

54. The method of claim 48, wherein the amount of any of said antigens is the same in different said chambers.

55. The method of claim 48, wherein each said chambers contains the same number, but proportionally different amounts of immobilized antigens.

56. The method of claim 55, further comprising identifying the amount of each of said immobilized antigens that gives rise to the highest apparent antibody affinity.

57. The method of claim 48, wherein each said chamber additionally contains one or more structurally similar analogs (e.g., nearest neighbor peptide antigens) for each said plurality of antigens.

58. An information database comprising:
(1) a plurality of PET sequences, and optionally one or more nearest neighbors of each of said PET sequences;
(2) property of antibodies specific for each of said PET sequences, said property including affinity towards said PET sequences, specificity towards said PET sequences against all other PET sequences and nearest neighbors, performance of each of said antibodies in one or more in vitro or in vivo assays.

59. A method of designing arrays for large scale profiling of analyte levels for a plurality of target analytes in a sample, the method comprising:
(1) generating one or more candidate capture agents specific for each of said target analytes or derivatives thereof;
(2) measuring the affinity and cross-reactivity of each of said candidate capture agents to select at least one capture agents with the highest specificity and/or fewest cross-reactivity for each of said target analytes or derivatives thereof;
(3) determining, based on the affinity of said at least one capture agents for their respective target analytes or derivatives thereof, and the normal abundance of soluble form of said target analytes or derivatives thereof in said sample, the amount of each of said target analytes or derivatives thereof for immobilization on a support;
wherein each said target analytes or derivatives thereof, when immobilized on said support in said amount, and when in contact with said sample, each produces substantially the same amount of binding to its capture agent.

60. The method of claim 59, wherein affinity is measured in step (2) by contacting said candidate capture agents with a concentration series of immobilized target analytes or derivatives thereof against which said candidate capture agents are raised.

61. The method of claim 59, wherein affinity for a plurality of candidate capture agents, each with different specificity, are simultaneously measured in step (2).

62. The method of claim 59, wherein cross-reactivity is measured in step (2) by contacting said candidate capture agents with one or more immobilized structurally similar homologs of target analytes or derivatives thereof against which said candidate capture agents are raised.

63. The method of claim 59, wherein cross-reactivity is measured in step (2) by using a proteome matrix array.

64. The method of claim 63, wherein said proteome matrix array comprises polypeptides representing each and every protein wthin the sample.

65. The method of claim 63, wherein said proteome matrix array comprises polypeptides representing the top 100, 300, 500, or 1000 most abundantly expressed proteins within the sample.

66. The method of claim 63, wherein said proteome matrix array excludes excessively hydrophobic peptides, short peptides of no more than 5 residues, or long peptides of no less than 50 residues.

67. The method of claim 63, wherein all peptides on said proteome matrix array have the same concentration.

68. The method of claim 63, wherein each peptide on said proteome matrix array has a concentration proportional to its concentration in the sample.

69. The method of claim 1, wherein the specificity value S for at least 50% of all of said capture agents is no more than about 0.1, preferably no more than about 0.05, 0.02, or 0.01.

70. The method of claim 59, further comprising manufacturing said array by immobilizing each of said target analytes or derivatives thereof in said amount determined in step (3).

71. The method of claim 59, wherein said sample is an undiluted serum sample, or a serum sample diluted by 2, 5, 10, 20, 50, 70, or 100 fold.

72. An array manufactured according to the method of claim 70.

73. A business method for a biotechnology or pharmaceutical business, the method comprising:
(1) designing, using the method of claim 59, an array with uniform dynamic range of measurements for each of the competent target analytes or derivatives thereof;
(2) licensing the right to further develop and/or manufacture said array to a third party.

74. A business method for a biotechnology or pharmaceutical business, the method comprising:
(1) designing, using the method of claim 59, an array of target analytes or derivatives thereof with uniform dynamic range of measurements for each of component said target analytes or derivatives thereof;
(2) manufacturing said array for use in diagnostic and/or research experimentation.

75. The business method of claim 74, further comprising marketing said arrays.

76. The business method of claim 74, further comprising distributing said arrays.

77. The business method of claim 74, wherein said arrays are for use in commercial and/or academic laboratories.

78. A method of screening for marker(s) associated with a condition, said method comprising:
(1) immobilizing a plurality of candidate analytes or fragments thereof, each on a series of distinct addressable locations, on a support;
(2) using competition assay and said immobilized candidate analytes, profiling the level of soluble forms of each of said candidate analytes in a panel of samples with said condition, and in a panel of corresponding control samples without said condition;
(3) identifying the candidate analyte(s), if any, as marker(s) associated with said condition, if the levels of soluble forms of said candidate analyte(s) in said panel of samples with said condition are significantly different from the levels of soluble forms of said candidate analyte(s) in said panel of control samples without said condition.

79. The method of claim 78, wherein said marker(s) are biomarkers representing surrogate endpoint(s).

80. The method of claim 78, wherein said condition is a disease condition, a condition associated with a treatment of a disease, or a condition associated with pollution.

81. The method of claim 78, wherein said analytes are small molecules with less than 5000 Da, or 3000 Da, 1000 Da, 500 Da, 100 Da, or 50 Da.

82. The method of claim 78, wherein said analytes are polypeptides, and said fragments are PET-containing peptide fragments.

83. The method of claim 78, wherein said analytes are mixtures of said small molecules of claim 81 and said polypeptides of claim 82.

84. The method of claim 78, further comprising manufacturing arrays comprising said marker(s) identified in (3).

85. The method of claim 84, wherein the levels of each of said marker(s) are statistically significantly different between said samples and said control samples.

86. The method of claim 84, wherein the levels of at least a few of said marker(s) are not statistically significantly different between said samples and said control samples.

87. An array of analytes constructed by the method of claim 84.

88. A method for quantitating a plurality of target analytes in a sample, comprising:
(1) for each of said plurality of target analytes or unique derivatives thereof, generating one or more capture agents that specifically bind said target analytes or said unique derivatives thereof, wherein said unique derivatives, if used, predictably result from a treatment of said plurality of target analytes within said sample;
(2) immobilizing said capture agents on a support, wherein each of said capture agent is immobilized on a series of distinct addressable locations on said support;
(3) optionally, subjecting said sample to said treatment;
(4) providing a mixture of standard analytes labeled with a first agent, each standard analyte has a predetermined concentration, and each standard analyte representing one of said target analytes, wherein all of said target analytes are represented by at least one of said standard analytes;
(5) labeling the target analytes in said sample with a second agent;
(6) contacting said capture agents to said mixture of standard analytes and said labeled target analytes in (5);
(7) measuring the amount of each pair of standard analyte and target analyte bound to their cognate capture agent on said support, thereby determining the amount of each of said target analytes in the sample, and/or the ratio of each target analyte compared to its corresponding standard analyte.
